(19) United States
(12) Patent Application Publication
(10) Pub. No.: US 2012/0053053 A1
(43) Pub. Date: Mar. 1, 2012

(54) PYRIMIDINE DERIVATIVES AND THEIR USE AS HERBICIDES

(75) Inventors: Mohamed Abdelouahab Boussemghoune, Berkshire (GB); William Guy Whittingham, Berkshire (GB); Caroline Louise Winn, Berkshire (GB); Harry Glithro, Berkshire (GB); Mary Bernadette Aspinall, Berkshire (GB)

(73) Assignee: SYNGENTA CROP PROTECTION, LLC, Greensboro, NC (US)

(21) Appl. No.: 13/201,101

(22) PCT Filed: Feb. 11, 2010
(86) PCT No.: PCT/GB2010/000242

§ 371 Date: Nov. 11, 2011

(30) Foreign Application Priority Data
Feb. 13, 2009 (GB) 0902474.6

(51) Int. Cl. A01N 43/54; C07D 239/42; C07D 239/38; C07D 239/34; C07D 239/52; A01P 13/00; C07C 257/18; A01N 25/32; C07D 239/28; C07D 405/10; C07D 239/30; C07D 239/56

(52) U.S. Cl. 504/103; 544/334; 544/329; 544/319; 544/312; 504/242; 504/239; 504/243; 564/244; 564/246; 564/247; 544/335; 544/328

(57) ABSTRACT

The present invention relates to substituted pyrimidine derivatives, as well as N-oxides thereof and agriculturally acceptable salts thereof, and their use to control undesired plant growth, in particular in crops of useful plants. The invention extends to herbicidal compositions comprising such compounds, N-oxides and/or salts as well as mixtures of the same with one or more further active ingredient (such as, for example, an herbicide, fungicide, insecticide and/or plant growth regulator) and/or a safener. The invention further relates to intermediates useful in the preparation of such compounds, and to processes for their preparation.

The present invention relates to substituted pyrimidine derivatives, as well as N-oxides thereof and agriculturally acceptable salts thereof, their use to control undesired plant growth, in particular in crops of useful plants, to processes for their preparation, and intermediates useful in such processes. The invention extends to herbicidal compositions comprising such compounds, N-oxides and/or salts as well as mixtures of the same with one or more further active ingredient (such as, for example, an herbicide, fungicide, insecticide and/or plant growth regulator) and/or a safener.

Substituted pyrimidine derivatives and their use as herbicides are disclosed in International Patent Publication No. WO 2005/063721. International Patent Publication No. WO 2007/082076 discloses a number of 2-(poly-substituted aryl)-6-amino-5-halo-4-pyrimidine carboxylic acids and their use as herbicides, whilst International Patent Publication No. WO 2007/092184 discloses certain substituted pyrimidine and pyridine carboxylic acid derivatives as compounds capable of improving the harvestability of crops. Further picolinic and pyrimidine acid derivatives are disclosed in WO2009/046090 and WO2009/029735.

In part, due to the evolution of herbicide-resistant weed populations, and herbicide-resistant crops becoming volunteer weeds, there is a continuing need to control such undesired plant growth in particular in crops of useful plants. Other factors, for example, the demand for cheaper, more effective herbicides, and for herbicides with an improved environmental profile (e.g. safer, less toxic etc.) also drive the need to identify novel herbicidal compounds.

The present invention seeks to address this need, and is based on the finding that substituted pyrimidine derivatives, in particular where the 5 position of the pyrimidine ring is substituted with an optionally substituted alkyl, optionally substituted cycloalkyl, optionally substituted alkenyl, or optionally substituted alkynyl group, are particularly effective herbicidal compounds.

Thus in a first aspect of the invention there is provided a compound of formula (I)

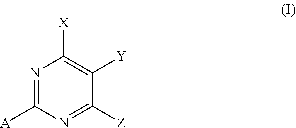

or salt or N-oxide thereof,

wherein:
A is halogen, optionally substituted alkylthio, optionally substituted alkyl, optionally substituted alkenyl or an optionally substituted 3-8 membered carbocyclic ring;
X is azido, halogen, optionally substituted alkoxy, optionally substituted alkylthio, optionally substituted alkylsulphinyl, optionally substituted alkylsulphonyl or NR⁵R⁶;
   R⁵ is hydrogen, C₂₋₄ alkenyl, SO₂R^{ss}, C(O)R^{uu} or optionally substituted C₁₋₄ alkyl;
   R⁶ is hydrogen, C₂₋₄ alkenyl or optionally substituted C₁₋₄ alkyl;
      each R^{ss} is independently C₁₋₄ alkyl or phenyl optionally substituted by 1-3 groups R^{zz};
      each R^{uu} is independently C₁₋₄ alkyl, phenyl optionally substituted by 1-3 groups R^{zz}, C₁₋₄ alkoxy, or NR^{ac}R^{ad};
      each R^{zz} is independently halogen, C₁₋₄ alkyl, C₁₋₄ alkoxy, or C₁₋₄ alkylsulphonyl;
      R^{ac} and R^{ad} are each independently hydrogen or C₁₋₄ alkyl;
   or R⁵ and R⁶ together form a group ═C(Rⁱ)OR^{j}, ═C(R^{k})SR^{l}, ═C(R^{m})NRⁿR^{o} wherein
      Rⁱ is hydrogen, C₁₋₄ alkyl, C₃₋₆ cycloalkyl, phenyl, C₁₋₄ alkoxy, C₁₋₄ alkylthio, or NR^{ac}R^{ad} wherein R^{ac} and R^{ad} are as defined above,
      R^{j} and R^{l} are each independently C₁₋₄ alkyl,
      R^{k} is hydrogen, C₁₋₄ alkyl, C₃₋₆ cycloalkyl, phenyl, C₁₋₄ alkylthio, or NR^{ac}R^{ad} wherein R^{ac} and R^{ad} are as defined above,
      R^{m} is hydrogen, C₁₋₄ alkyl, C₃₋₆ cycloalkyl, phenyl, or NR^{ac}R^{ad} wherein R^{ac} and R^{ad} are as defined above, and
      Rⁿ and R^{o} are each independently hydrogen or C₁₋₄ alkyl;
Y is optionally substituted alkyl, optionally substituted cycloalkyl, optionally substituted alkenyl, or optionally substituted alkynyl;
and,
Z is Oₘ—(CHR^{w})ₙ—C(O)R^{cb}, wherein
   m is an integer of 0 or 1,
   n is an integer of 0 or 1 and n≧m,
   R^{w} is hydrogen or C₁₋₄ alkyl
   R^{cb} is hydroxy, optionally substituted alkylthio, NH₂, or OR^{co},
   R^{co} is C₁₋₂₀ alkyl optionally substituted by 1-3 groups R^{cq}, or C₁₋₂₀ haloalkyl optionally substituted by 1-3 groups R^{cq};
   each R^{cq} is independently C₁₋₆ alkoxy, phenyl optionally substituted by 1-3 groups R^{cr}, or heteroaryl optionally substituted by 1-2 groups R^{cs};
   each R^{cr} and each R^{cs} are independently halogen, cyano, C₁₋₄ alkyl, C₁₋₄ haloalkyl, C₂₋₆ alkoxyalkyl, C₁₋₄ alkoxy, C₁₋₄ haloalkoxy, C₁₋₄ alkylsulphonyl, or C₁₋₄ alkoxycarbonyl;
   provided that:
   i) when Y is C₁₋₄ alkyl, C₁₋₄ haloalkyl, C₂₋₄ alkoxyalkyl, C₂₋₄ alkylthioalkyl, C₂₋₄ alkenyl, C₂₋₄ haloalkenyl, C₂₋₄ alkoxyalkenyl, C₂₋₄ thioalkylalkenyl, C₂₋₄ alkynyl, C₂₋₄ haloalkynyl, C₂₋₄ alkylcarbonyl or C₂₋₄ haloalkylcarbonyl;
   X is NR⁵R⁶;
   R⁵ is H, C₁₋₄ alkyl, C₃₋₄ alkenyl, C₁₋₄ alkylsulfonyl or C₁₋₄ acyl;
   R⁶ is H, C₁₋₄ alkyl or C₃₋₄ alkenyl;
   A is C1-C6 alkyl, cyclopropyl, C1-C6 haloalkyl, C2-C6 alkenyl, C2-C6 haloalkenyl or a group of the formula

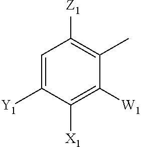

W₁ represents H or halogen;
X₁ represents H, halogen, nitro, cyano, formyl, C₁₋₆ alkyl, C₂₋₆ alkenyl, C₂₋₆ alkynyl, C₁₋₆ alkoxy, C₂₋₄ alkoxyalkyl, C₁₋₆ alkylcarbonyl, C₁₋₆ alkylthio, C₁₋₆ alkylsulfinyl, C₁₋₆ alkylsulfonyl, C₂₋₄ alkenyloxy, C₂₋₄ alkynyloxy, C₂₋₄ alkenylthio, C₂₋₄ alkynylthio, C₁₋₆ haloalkyl, C₂₋₆ halo-alkenyl, C₂₋₆ haloalkynyl, C₁₋₆ haloalkoxy, C₂₋₄ haloalkoxyalkyl, C₂₋₆ haloalkylcarbonyl, C₁₋₆ haloalkylthio, C₁₋₆ haloalkylsulfinyl, C₁₋₆ halo-.alkylsulfonyl, C₃₋₆ trialkylsilyl, C₂₋₄ haloalkenyloxy, C₂₋₄ haloalkynyloxy, C₂₋₄ haloalkenylthio, C₂₋₄ haloalkynylthio, —C(O)OR^{7′}, —C(O)NR^{6′}R^{7′}, —CR^{6′}NOR^{7′}, —NR^{6′}R^{7′}, —NR^{6′}OR^{7′}, —NR^{6′}SO2R^{7′}, —NR^{6′}C(O)R^{7′}, —NR^{6′}(O)OR^{7′}, —NR^{6′}C(O)NR^{6′}R^{7′} or —NCR^{6′}NR^{6′}R^{7′};
   R^{6′} represents H, C₁₋₄ alkyl or C₁₋₄ haloalkyl;
   R^{7′} represents C₁₋₄ alkyl or C₁₋₄ haloalkyl;
   Y₁ represents H, halogen, C₁₋₆ alkyl, C₁₋₆ haloalkyl, C₁₋₆ alkoxy, C₁₋₆ haloalkoxy, C₂₋₆ alkenyl or C₂₋₆ haloalkenyl, or, X₁ and Y₁ taken together, represents —O(CH₂)ₙₙCH₂—, or —O(CH₂)ₙₙO— wherein nn=1 or 2; and
   Z₁ represents H or halogen, then
      Z is other than CO₂H, CO₂Me, CO₂Et, CO₂ⁿBu or CO₂⁻NHEt₃⁺.
      In a second aspect of the invention there is provided a herbicidal composition comprising a compound as defined above and at least one agriculturally acceptable formulation adjuvant or diluent.

In a second aspect of the invention there is provided a compound as defined above, or a herbicidal composition as defined above in admixture with at least one active ingredient selected from the group consisting of: an insecticide, an acaricide, a nematocide, a molluscicide, an herbicide, a fungicide, and a plant growth regulator.

In a third aspect of the invention there is provided a use of a compound according to the invention as a herbicide.

In a fourth aspect of the invention, there is provided a method of controlling weeds in crops of useful plants which comprises applying to said weeds or to the locus of said weeds, or to said crop of useful plants, a compound, composition or a mixture according to the invention.

In a fifth aspect of the invention, there is provided a compound of formula (II)

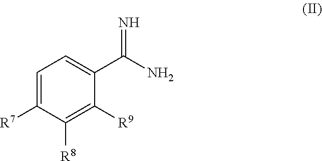

wherein
R⁷ is methyl, Br, or Cl;

### R⁸ is H, F, Cl, OR¹⁰, or N(R¹⁰)₂;

### R⁹ is H, F, or Cl; and

each R¹⁰ is independently H or C₁₋₄alkyl,
provided that
(i) R⁸ and R⁹ are not both hydrogen,
(ii) when R⁷ is methyl and R⁹ is hydrogen, then R⁸ is not F, Cl, or NH₂,
(iii) when R⁷ is Cl and R⁸ is hydrogen, R⁹ is not Cl,
(iv) when R⁷ is Cl and R⁸ is Cl, R⁹ is not hydrogen, and
(v) when R⁷ is Br and R⁹ is hydrogen, R⁸ is not F.

In a sixth aspect of the invention, there is provided a process for the preparation of a compound of formula (T)

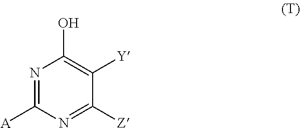

or salt or N-oxide thereof,

wherein:
A is halogen, optionally substituted alkylthio, optionally substituted alkyl, optionally substituted alkenyl or an optionally substituted 3-8 membered carbocyclic ring;
Y′ is hydrogen, optionally substituted alkyl, optionally substituted cycloalkyl, optionally substituted alkenyl, or optionally substituted alkynyl;
and,
Z′ is hydroxyl or (CHR^{w})ₙ—C(O)R^{cb}, wherein
   n is an integer of 0 or 1,
   R^{w} is hydrogen or C₁₋₄ alkyl
   R^{cb} is hydroxy, optionally substituted alkylthio, optionally substituted alkyl, NH₂, or OR^{co},
   R^{co} is C₁₋₂₀ alkyl optionally substituted by 1-3 groups R^{cq}, or C₁₋₂₀ haloalkyl optionally substituted by 1-3 groups R^{cq};
   each R^{cq} is independently C1-6 alkoxy, phenyl optionally substituted by 1-3 groups R^{cr}, or heteroaryl optionally substituted by 1-2 groups R^{cs};
      each R^{cr} and each R^{cs} are independently halogen, cyano, C₁₋₄ alkyl, C₁₋₄ haloalkyl, C₂₋₆ alkoxyalkyl, C₁₋₄ alkoxy, C₁₋₄ haloalkoxy, C₁₋₄ alkylsulphonyl, or C₁₋₄ alkoxycarbonyl comprising reacting an amidine of formula (N)

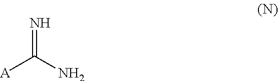

wherein A is as defined above, or a salt form thereof, with a keto ester of the formula (U)

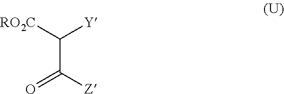

or a salt form thereof,
wherein Y′ and Z′ are as defined above, or Z′ is OR, and R is selected from hydrogen or alkyl.

In an seventh aspect, there is provided a compound of formula (I)

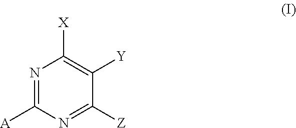

or salt or N-oxide thereof,

wherein:
A is halogen, optionally substituted alkylthio, optionally substituted alkyl, optionally substituted alkenyl or an optionally substituted 3-8 membered carbocyclic ring;
X is azido, halogen, optionally substituted alkoxy, or NR⁵R⁶;
   R⁵ is hydrogen, optionally substituted C₁₋₄ alkyl provided said substitution does not comprise a ring system or a hydroxyl group, C₂₋₄ alkenyl, SO₂R^{ss}, or C(O)R^{uu};
   R⁶ is hydrogen, optionally substituted C₁₋₄ alkyl provided said substitution does not comprise a ring system or a hydroxyl group, C₂₋₄ alkenyl;
      each R^{ss} is independently C₁₋₄ alkyl or phenyl optionally substituted by 1-3 groups R^{zz};
      each R^{uu} is independently C₁₋₄ alkyl, phenyl optionally substituted by 1-3 groups R^{zz}, C₁₋₄ alkoxy, or NR^{ac}R^{ad};
      each R^{zz} is independently halogen, C₁₋₄ alkyl, C₁₋₄ alkoxy, or C₁₋₄ alkylsulphonyl;
      R^{ac} and R^{ad} are each independently hydrogen or C₁₋₄ alkyl;
   or R⁵ and R⁶ together form a group ═C(Rⁱ)OR^{j}, ═C(R^{k})SR^{l}, ═C(R^{m})NRⁿR^{o} wherein
      Rⁱ is hydrogen, C₁₋₄ alkyl, C₃₋₆ cycloalkyl, phenyl, C₁₋₄ alkoxy, C₁₋₄ alkylthio, or NR^{ac}R^{ad} wherein R^{ac} and R^{ad} are as defined above,
      R^{j} and R^{l} are each independently C₁₋₄ alkyl,
      R^{k} is hydrogen, C₁₋₄ alkyl, C₃₋₆ cycloalkyl, phenyl, C₁₋₄ alkylthio, or NR^{ac}R^{ad} wherein R^{ac} and R^{ad} are as defined above,
      R^{m} is hydrogen, C₁₋₄ alkyl, C₃₋₆ cycloalkyl, phenyl, or NR^{ac}R^{ad} wherein R^{ac} and R^{ad} are as defined above, and
      Rⁿ and R^{o} are each independently hydrogen or C₁₋₄ alkyl;
Y is optionally substituted alkyl, optionally substituted cycloalkyl, optionally substituted alkenyl, or optionally substituted alkynyl;
and,
Z is Oₘ—(CHR^{w})ₙ—C(O)R^{cb}, wherein
   m is an integer of 0 or 1,
   n is an integer of 0 or 1 and n≧m,
   R^{w} is hydrogen or C₁₋₄ alkyl
   R^{cb} is hydroxy, optionally substituted alkylthio, optionally substituted alkyl, NH₂, or OR^{co},
   R^{co} is C₁₋₂₀ alkyl optionally substituted by 1-3 groups R^{cq}, or C₁₋₂₀ haloalkyl optionally substituted by 1-3 groups R^{cq};
   each R^{cq} is independently phenyl optionally substituted by 1-3 groups R^{cr}, or heteroaryl optionally substituted by 1-2 groups R^{cs};
      each R^{cr} and each R^{cs} are independently halogen, cyano, C₁₋₄ alkyl, C₁₋₄ haloalkyl, C₂₋₆ alkoxyalkyl, C₁₋₄ alkoxy, C₁₋₄ haloalkoxy, C₁₋₄ alkylsulphonyl, or C₁₋₄ alkoxycarbonyl.

For the avoidance of doubt, the term “compound” as used herein includes all salts and N-oxides of said compound.

The compounds of formula (I) may exist in different geometric or optical isomers or different tautomeric forms. One or more centres of chirality may be present, in which case compounds of the formula (I) may be present as pure enantiomers, mixtures of enantiomers, pure diastereomers or mixtures of diastereomers. There may be double bonds present in the molecule, such as C═C or C═N bonds, in which case compounds of formula (I) may exist as single isomers or mixtures of isomers. Centres of tautomerisation may be present. This invention covers all such isomers and tautomers and mixtures thereof in all proportions as well as isotopic forms such as deuterated compounds.

Suitable acid addition salts include those with an inorganic acid such as hydrochloric, hydrobromic, sulfuric, nitric and phosphoric acids, or an organic carboxylic acid such as oxalic, tartaric, lactic, butyric, toluic, hexanoic and phthalic acids, or sulphonic acids such as methane, benzene and toluene sulphonic acids. Other examples of organic carboxylic acids include haloacids such as trifluoroacetic acid.

Suitable salts also include those formed by strong bases (e.g. metal hydroxides—in particular sodium, potassium or lithium—or quaternary ammonium hydroxide) as well as those formed with amines.

N-oxides are oxidised forms of tertiary amines or oxidised forms of nitrogen containing heteroaromatic compounds. They are described in many books for example in “Heterocyclic N-oxides” by Angelo Albini and Silvio Pietra, CRC Press, Boca Raton, Fla., 1991.

Each alkyl moiety either alone or as part of a larger group (such as alkoxy, alkoxyalkyl, alkylcarbonyl, alkoxycarbony, alkylaminocarbonyl, alkylsulphonyl, alkylthio, hakoalkylthio, haloalkyl, haloalkoxy, trialklylsilyl, etc.) is a straight or branched chain and is, for example, methyl, ethyl, n-propyl, n-butyl, n-pentyl, n-hexyl, iso-propyl, sec-butyl, iso-butyl, tert-butyl or neo-pentyl. As defined herein alkyl groups may be C₁ to C₂₀ alkyl groups, but are typically C₁₋₁₀, preferably C₁-C₆, more preferably C₁-C₄, even more preferably C₁₋₃, and most preferably C₁-C₂ alkyl groups.

Ring or chain forming alkylene, alkenylene and alkynylene groups can optionally be further substituted by one or more halogen, C₁₋₃ alkyl and/or C₁₋₃ alkoxy groups.

When present, the optional substituents on an alkyl moiety (alone or as part of a larger group) include one or more of halogen, nitro, cyano, C₃₋₇ cycloalkyl (itself optionally substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, C₁₋₆ alkoxycarbonyl(C₁₋₁₀)alkoxy, C₁₋₁₀ haloalkoxy, aryl(C₁₋₄-alkoxy (where the aryl group is optionally substituted), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, mercapto, C₁₋₁₀ alkylthio, C₁₋₁₀ haloalkylthio, aryl(C₁₋₄alkylthio (where the aryl group is optionally substituted), C₃₋₇ cycloalkylthio (where the cycloalkyl group is optionally substituted with C₁₋₆ alkyl or halogen), arylthio (where the aryl group is optionally substituted), C₁₋₆ alkylsulfonyl, C₁₋₆ haloalkylsulfonyl, C₁₋₆ alkylsulfinyl, C₁₋₆ haloalkylsulfinyl, arylsulfonyl (where the aryl group may be optionally substituted), tri(C₁₋₄)alkylsilyl, aryl(C₁₋₄)alkylthio(C₁₋₄)alkyl, aryloxy(C₁₋₄)alkyl, formyl, C₁₋₁₀ alkylcarbonyl, HO₂C, C₁₋₁₀ alkoxycarbonyl, aminocarbonyl, C₁₋₆ alkylaminocarbonyl, di(C₁₋₆ alkyl)aminocarbonyl, N—(C₁₋₃ alkyl)-N—(C₁₋₃ alkoxy)aminocarbonyl, C₁₋₆ alkylcarbonyloxy, arylcarbonyloxy (where the aryl group is optionally substituted), di(C₁₋₆)alkylaminocarbonyloxy, oximes and oxime-ethers such as ═NOalkyl, ═NOhaloalkyl and ═NOaryl (itself optionally substituted), aryl (itself optionally substituted), heteroaryl (itself optionally substituted), heterocyclyl (itself optionally substituted with C₁₋₆ alkyl or halogen), aryloxy (where the aryl group is optionally substituted), heteroaryloxy, (where the heteroaryl group is optionally substituted), heterocyclyloxy (where the heterocyclyl group is optionally substituted with C₁₋₆ alkyl or halogen), amino, C₁₋₆ alkylamino, di(C₁₋₆)alkylamino, C₁₋₆ alkylcarbonylamino, N—(C₁₋₆)alkylcarbonyl-N—(C₁₋₆)alkylamino, C₂₋₆ alkenylcarbonyl, C₂₋₆ alkynylcarbonyl, C₃₋₆ alkenyloxycarbonyl, C₃₋₆ alkynyloxycarbonyl, aryloxycarbonyl (where the aryl group is optionally substituted) and arylcarbonyl (where the aryl group is optionally substituted).

Alkenyl and alkynyl moieties can be in the form of straight or branched chains, and the alkenyl moieties, where appropriate, can be of either the (E)- or (Z)-configuration. Preferably alkenyl moieties are C₂₋₆ alkenyl groups, more preferably C₂₋₄ alkenyl groups, and most preferably vinyl or allyl. Alkynyl moieties are preferably C₂₋₆ alkynyl groups, more preferably C₂₋₄ alkynyl groups and most prefereably ethynyl and propargyl. Alkenyl and alkynyl moieties can contain one or more double and/or triple bonds in any combination. It is understood, that allenyl and alkynylalkenyl are included in these terms.

When present, the optional substituents on alkenyl or alkynyl include those optional substituents given above for an alkyl moiety.

In the context of this specification acyl is optionally substituted C₁₋₆ alkylcarbonyl (for example acetyl), optionally substituted C₂₋₆ alkenylcarbonyl, optionally substituted C₃₋₆ cycloalkylcarbonyl (for example cyclopropylcarbonyl), optionally substituted C₂₋₆ alkynylcarbonyl, optionally substituted arylcarbonyl (for example benzoyl) or optionally substituted heteroarylcarbonyl.

Halogen is fluorine, chlorine, bromine or iodine.

Haloalkyl groups are alkyl groups which are substituted with one or more of the same or different halogen atoms and are, for example, CF₃, CF₂Cl, CF₂H, CCl₂H, FCH₂, ClCH₂, BrCH₂, CH₃CHF, (CH₃)₂CF, CF₃CH₂ or CHF₂CH₂.

In the context of the present specification ring systems may be saturated, unsaturated, or aromatic, and may also be fused, spiro or bridging ring systems. The terms “aryl”, “aromatic ring” and “aromatic ring system” as used herein refer to ring systems which may be mono-, bi- or tricyclic. Examples of such rings include phenyl, naphthalenyl, anthracenyl, indenyl or phenanthrenyl. A preferred aryl group is phenyl. In addition, the terms “heteroaryl”, “heteroaromatic ring” or “heteroaromatic ring system” refer to an aromatic ring system containing at least one heteroatom and consisting either of a single ring or of two or more fused rings. Preferably, single rings will contain up to three and bicyclic systems up to four heteroatoms which will preferably be chosen from nitrogen, oxygen and sulphur. Examples of such groups include furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl. Preferred examples of heteroaromatic radicals include pyridyl, pyrimidyl, triazinyl, thienyl, furyl, oxazolyl, isoxazolyl and thiazolyl.

The terms heterocycle and heterocyclyl refer to a non-aromatic preferably monocyclic or bicyclic ring systems containing up to 10 atoms including one or more (preferably one or two) heteroatoms selected from O, S and N. Examples of such rings include 1,3-dioxolane, oxetane, tetrahydrofuran, morpholine, thiomorpholine and piperazine.

In the case of heteroaromatic or heterocyclic rings containing S as a heteroatom, the S atom may also be in the form of a mono- or di-oxide.

When present, the optional substituents on heterocyclyl include C₁₋₆ alkyl and C₁₋₆ haloalkyl, an oxo-group (allowing one of the carbon atoms in the ring to be in the form of a keto group), as well as those optional substituents given above for an alkyl moiety.

Cycloalkyl includes cyclopropyl, cyclobutyl, cyclopentyl and cyclohexyl. Cycloalkylalkyl is preferentially cyclopropylmethyl. Cycloalkenyl includes cyclopentenyl and cyclohexenyl.

When present, the optional substituents on cycloalkyl or cycloalkenyl include C₁₋₃ alkyl as well as those optional substituents given above for an alkyl moiety.

Carbocyclic rings include aryl, cycloalkyl and cycloalkenyl groups.

When present, the optional substituents on aryl or heteroaryl are selected independently, from halogen, nitro, cyano, C₁₋₆ alkyl, C₁₋₆ haloalkyl, C₁₋₆ alkoxy-(C₁₋₆)alkyl, C₂₋₆ alkenyl, C₂₋₆ haloalkenyl, C₂₋₆ alkynyl, C₃₋₇ cycloalkyl (itself optionally substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄)alkyl-silyl(C₁₋₆)alkoxy, C₁₋₆ alkoxycarbonyl(C₁₋₁₀)alkoxy, C₁₋₁₀ haloalkoxy, aryl(C₁₋₄)alkoxy (where the aryl group is optionally substituted with halogen or C₁₋₆ alkyl), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, mercapto, C₁₋₁₀ alkylthio, C₁₋₁₀ haloalkylthio, aryl(C₁₋₄)alkylthio, C₃₋₇ cycloalkylthio (where the cycloalkyl group is optionally substituted with C₁₋₆ alkyl or halogen), tri(C₁₋₄-alkylsilyl(C₁₋₆)alkylthio, arylthio, C₁₋₆ alkylsulfonyl, C₁₋₆ haloalkylsulfonyl, C₁₋₆ alkylsulfinyl, C₁₋₆ haloalkylsulfinyl, arylsulfonyl, tri(C₁₋₄)alkylsilyl, aryldi(C₁₋₄-alkylsilyl, (C₁₋₄)alkyldiarylsilyl, triarylsilyl, C₁₋₁₀ alkylcarbonyl, HO₂C, C₁₋₁₀ alkoxycarbonyl, aminocarbonyl, C₁₋₆ alkylaminocarbonyl, di(C₁₋₆ alkyl)-aminocarbonyl, N—(C₁₋₃ alkyl)-N—(C₁₋₃ alkoxy)aminocarbonyl, C₁₋₆ alkylcarbonyloxy, arylcarbonyloxy, di(C₁₋₆)alkylamino-carbonyloxy, aryl (itself optionally substituted with C₁₋₆ alkyl or halogen), heteroaryl (itself optionally substituted with C₁₋₆ alkyl or halogen), heterocyclyl (itself optionally substituted with C₁₋₆ alkyl or halogen), aryloxy (where the aryl group is optionally substituted with C₁₋₆ alkyl or halogen), heteroaryloxy (where the heteroaryl group is optionally substituted with C₁₋₆ alkyl or halogen), heterocyclyloxy (where the heterocyclyl group is optionally substituted with C₁₋₆ alkyl or halogen), amino, C₁₋₆ alkylamino, di(C₁₋₆)alkylamino, C₁₋₆ alkylcarbonylamino, N—(C₁₋₆)alkylcarbonyl-N—(C₁₋₆)alkylamino, arylcarbonyl, (where the aryl group is itself optionally substituted with halogen or C₁₋₆ alkyl) or two adjacent positions on an aryl or heteroaryl system may be cyclised to form a 5, 6 or 7 membered carbocyclic or heterocyclic ring, itself optionally substituted with halogen or C₁₋₆ alkyl. Further substituents for aryl or heteroaryl include arylcarbonylamino (where the aryl group is substituted by C₁₋₆ alkyl or halogen), C₁₋₆alkoxycarbonylamino, C₁₋₆alkoxycarbonyl-N—(C₁₋₆)alkylamino, aryloxycarbonylamino (where the aryl group is substituted by C₁₋₆ alkyl or halogen), aryloxycarbonyl-N—(C₁₋₆)alkylamino (where the aryl group is substituted by C₁₋₆ alkyl or halogen), arylsulphonylamino (where the aryl group is substituted by C₁₋₆ alkyl or halogen), arylsulphonyl-N—(C₁₋₆)alkylamino (where the aryl group is substituted by C₁₋₆ alkyl or halogen), aryl-N—(C₁₋₆)alkylamino (where the aryl group is substituted by C₁₋₆ alkyl or halogen), arylamino (where the aryl group is substituted by C₁₋₆ alkyl or halogen), heteroarylamino (where the heteroaryl group is substituted by C₁₋₆ alkyl or halogen), heterocyclylamino (where the heterocyclyl group is substituted by C₁₋₆ alkyl or halogen), aminocarbonylamino, C₁₋₆ alkylaminocarbonylamino, di(C₁₋₆)alkylaminocarbonylamino, arylaminocarbonylamino where the aryl group is substituted by C₁₋₆ alkyl or halogen), aryl-N—(C₁₋₆)alkylaminocarbonylamino (where the aryl group is substituted by C₁₋₆ alkyl or halogen), C₁₋₆ alkylaminocarbonyl-N—(C₁₋₆)alkylamino, di(C₁₋₆)alkylaminocarbonyl-N—(C₁₋₆)alkylamino, arylaminocarbonyl-N—(C₁₋₆)alkylamino (where the aryl group is substituted by C₁₋₆ alkyl or halogen) and aryl-N—(C₁₋₆)alkylaminocarbonyl-N—(C₁₋₆)alkylamino (where the aryl group is substituted by C₁₋₆ alkyl or halogen).

For substituted phenyl moieties, heterocyclyl and heteroaryl groups it is preferred that one or more substituents are independently selected from halogen, C₁₋₆ alkyl, C₁₋₆ haloalkyl, C₁₋₆ alkoxy(C₁₋₆)alkyl, C₁₋₆ alkoxy, C₁₋₆ haloalkoxy, C₁₋₆ alkylthio, C₁₋₆ haloalkylthio, C₁₋₆ alkylsulfinyl, C₁₋₆ haloalkylsulfinyl, C₁₋₆ alkylsulfonyl, C₁₋₆ haloalkylsulfonyl, C₂₋₆ alkenyl, C₂₋₆ haloalkenyl, C₂₋₆ alkynyl, C₃₋₇ cycloalkyl, nitro, cyano, CO₂H, C₁₋₆ alkylcarbonyl, C₁₋₆ alkoxycarbonyl, aryl, heteroaryl, C₁₋₆ alkylamino, di(C₁₋₆ alkyl)amino, C₁₋₆ alkylaminocarbonyl, or di(C₁₋₆ alkyl)aminocarbonyl.

Haloalkenyl groups are alkenyl groups which are substituted with one or more of the same or different halogen atoms.

It is to be understood that dialkylamino substituents include those where the dialkyl groups together with the N atom to which they are attached form a five, six or seven-membered heterocyclic ring which may contain one or two further heteroatoms selected from O, N or S and which is optionally substituted by one or two independently selected (C₁₋₆)alkyl groups. When heterocyclic rings are formed by joining two groups on an N atom, the resulting rings are suitably pyrrolidine, piperidine, thiomorpholine and morpholine each of which may be substituted by one or two independently selected (C₁₋₆) alkyl groups.

In preferred embodiments of the invention, the preferred groups for A, X, Y, and Z, in any combination thereof, are as set out below.

According to the invention A is selected from halogen, optionally substituted alkylthio, optionally substituted alkyl, optionally substituted alkenyl and an optionally substituted 3-8 membered carbocyclic ring. Preferably, A is optionally substituted alkylthio.

In preferred embodiments A is selected from: halogen, C₁₋₄ alkylthio, phenyl optionally substituted by 1-3 groups R¹, and C₃₋₆ cycloalkyl optionally substituted by 1-4 groups R².

More preferably A is Cl, phenyl optionally substituted by 1-3 groups R¹, or cyclopropyl optionally substituted by 1-2 groups R².

Most preferably A is selected from the group consisting of: Cl, methylthio, isopropyl, cyclopropyl, 2-methylcyclopropyl, 4-methylphenyl, 4-methoxyphenyl, 4-chlorophenyl, 4-bromophenyl, 4-iodophenyl, 4-fluorophenyl, 4-trifluoromethylphenyl, 4-trifluoromethoxyphenyl, 2,4-dimethoxyphenyl, 2,4-dichlorophenyl, 2-chloro-4-methylphenyl, 2-chloro-4-trifluoromethylphenyl, 2-fluoro-4-methylphenyl, 2-fluoro-3-methoxyphenyl, 2-fluoro-4-methoxyphenyl, 2,4-bis(trifluoromethyl)phenyl, 3,4-dimethylphenyl, 3,4-dimethoxyphenyl, 3-chloro-4-methylphenyl, 3-chloro-4-methoxyphenyl, 3,4-dichlorophenyl, 3-chloro-4-fluorophenyl, 3-chloro-4-trifluoromethylphenyl, 3-fluoro-4-methylphenyl, 3-fluoro-4-methoxyphenyl, 4-methyl-3-nitrophenyl, 4-methoxy-2-methylphenyl, 4-chloro-2-methylphenyl, 4-chloro-3-methylphenyl, 4-chloro-2-methoxyphenyl, 4-chloro-3-methoxyphenyl, 4-chloro-3-nitrophenyl, 4-chloro-3-cyanophenyl, 4-chloro-2-fluorophenyl, 4-chloro-3-fluorophenyl, 4-chloro-2-trifluoromethylphenyl, 4-chloro-3-trifluoromethylphenyl, 4-fluoro-3-methylphenyl, 4-fluoro-3-methoxyphenyl, 4-fluoro-3-trifluoromethylphenyl, 2,4,5-trimethylphenyl, 2,3,4-trimethoxyphenyl, 2,3,4-trichlorophenyl, 2,4,5-trichlorophenyl, 2,4,6-trichlorophenyl, 2,3,4-trifluorophenyl, 2,4-dichloro-3-fluorophenyl, 3,4-dichloro-2-fluorophenyl, 4-chloro-2,3-difluorophenyl, 4-chloro-2,6-difluorophenyl, 4-chloro-3,5-difluorophenyl, 2,4-dichloro-6-fluorophenyl, 4-chloro-2-fluoro-3-methoxyphenyl, 4-chloro-2-fluoro-3-trifluoromethylphenyl, 4-chloro-3-dimethylamino-2-fluorophenyl, and 2-fluoro-3-methoxy-4-methylphenyl.

Each R¹ is independently halogen, cyano, C₁₋₄ alkyl, C₁₋₄ haloalkyl, C₁₋₄ alkoxy, C₁₋₄ haloalkoxy, C₁₋₄ alkylthio, C₁₋₄ haloalkylthio, amino, C₁₋₄ alkylamino, di(C₁₋₄)alkylamino, or two adjacent groups R¹ together with the atoms to which they are joined form a 6-membered aromatic ring, said ring being optionally substituted by 1-2 groups selected from: halogen, cyano, C₁₋₄ alkyl, C₁₋₄ haloalkyl, C₁₋₄ alkoxy, C₁₋₄ haloalkoxy, C₁₋₄ alkylthio, C₁₋₄ haloalkylthio. Preferably each R¹ is independently halogen, cyano, C₁₋₂ alkyl, C₁₋₂ haloalkyl, C₁₋₂ alkoxy, C₁₋₂ haloalkoxy, amino, C₁₋₄ alkylamino, or di(C₁₋₄)alkylamino.

Each R² is independently halogen, cyano, C₁₋₄ alkyl, C₁₋₄ haloalkyl, C₃₋₆ cycloalkyl, C₁₋₄ alkoxy, C₁₋₄ haloalkoxy, C₁₋₄ alkoxycarbonyl, or C₁₋₄ alkylaminocarbonyl; or any two geminal R² groups together form a group selected from oxo, ═CR^{mm}Rⁿⁿ, or ═NOR^{oo}; or two groups R² together with the atoms to which they are joined form a 3-6-membered ring system, said ring system being optionally substituted by 1-2 groups selected from: halogen, cyano, C₁₋₄ alkyl, C₁₋₄ haloalkyl, C₁₋₄ alkoxy, or C₁₋₄ haloalkoxy. Preferably R² is halogen, cyano, C₁₋₂ alkyl, C₁₋₂ haloalkyl, C₁₋₂ alkoxy, C₁₋₂ haloalkoxy or C₂₋₄ alkoxycarbonyl. R^{mm} and Rⁿⁿ are each independently hydrogen, halogen, cyano, nitro, C₁₋₄ alkyl, or C₁₋₄ alkoxycarbonyl. R^{oo} is hydrogen, C₁₋₄ alkyl, C₃₋₆ cycloalkyl(C₁₋₂)alkyl or C₃₋₆ cycloalkyl.

According to the invention X is selected from: azido, halogen, optionally substituted alkoxy, optionally substituted alkylthio, optionally substituted alkylsulphinyl, optionally substituted alkylsulphonyl and NR⁵R⁶, where R⁵ and R⁶ are as defined hereinbefore.

In preferred embodiments X is selected from: azido, halogen, C₁₋₃ alkoxy, C₁₋₄ alkoxycarbonylC₁₋₃alkoxy, and NR⁵R⁶. Most preferably X is N₃, Cl, OCH₃, OCH₂CO₂CH₃, NH₂, NHCH₃, N(CH₃)₂, NH-isopropyl, NHCOCH₃, NHC(O)OCH₃, NHSO₂CH₃, NCH₃COCH₃, NCH₃C(O)OCH₃, or NCH₃SO₂CH₃.

In alternative preferred embodiments, X is selected from: azido, halogen, optionally substituted alkoxy, optionally substituted alkylthio, optionally substituted alkylsulphinyl and optionally substituted alkylsulphonyl. More preferably, X is selected from: optionally substituted alkylthio, optionally substituted alkylsulphinyl and optionally substituted alkylsulphonyl.

Preferably R⁵ is hydrogen, C₁₋₄ alkyl optionally substituted with 1 or 2 hydroxy or C₁₋₄ alkoxy groups, C₁₋₄ haloalkyl optionally substituted with 1 or 2 hydroxy or C₁₋₄ alkoxy groups, C₂₋₄ alkenyl, SO₂R^{ss}, or C(O)R^{uu}, wherein R^{ss} and R^{uu} are as defined hereinbefore. In more preferred embodiments, R^{ss} and R^{uu} are each independently C₁₋₃ alkyl.

Preferably R⁶ is hydrogen, C₁₋₄ alkyl optionally substituted with 1 or 2 hydroxy or C₁₋₄ alkoxy groups, C₁₋₄ haloalkyl optionally substituted with 1 or 2 hydroxy or C₁₋₄ alkoxy groups, or C₂₋₄ alkenyl.

Alternatively, and preferably, R⁵ is hydrogen, C₂₋₄ alkenyl, SO₂R^{ss}, C(O)R^{uu} or optionally substituted C₁₋₄ alkyl provided said substitution does not comprise a ring system or a hydroxyl group;

Alternatively, and preferably, R⁶ is hydrogen, C₂₋₄ alkenyl or optionally substituted C₁₋₄ alkyl provided said substitution does not comprise a ring system or a hydroxyl group;

In preferred embodiments of the present invention Y is C₁₋₆ alkyl optionally substituted by 1-3 groups R^{ba}, C₁₋₆ haloalkyl optionally substituted by 1-3 groups R^{ba}, C₃₋₆ cycloalkyl optionally substituted by 1-3 groups R^{bc}, C₂₋₆alkenyl optionally substituted by 1-3 groups R^{bd}, or C₂₋₆ alkynyl optionally substituted by 1-3 groups R^{be}.

In more preferred embodiments Y is C₁₋₃ alkyl, C₁₋₃ haloalkyl, C₂₋₅alkoxyalkyl, cyclopropyl optionally substituted by 1 or 2 groups R^{bc}, C₂₋₄ alkenyl, C₂₋₄ haloalkenyl, or C₂₋₄ alkynyl optionally substituted by 1 or 2 groups R^{be}.

More preferably still, Y is selected from the group consisting of: methyl, ethyl, isopropyl, n-propyl, prop-1-en-2-yl, prop-1-enyl, prop-2-enyl, but-1-enyl, pent-1-enyl, difluoromethyl, trifluoromethyl, hydroxymethyl, 1-hydroxyethyl, 2-hydroxyethyl, 1-methylprop-1-enyl, 2-methylprop-1-enyl, 1,2-dimethylprop-1-enyl, 3-methylbut-1-ynyl, 3-methylbut-2-enyl, 3,3-dimethylbut-1-ynyl, acetyl, formyl, methoxymethyl, 2-methoxyethyl, hydroxyiminomethyl, methoxyiminomethyl, 1-(hydroxyimino)ethyl, 1-(methoxyimino)ethyl, cyclopropyl, 1-methylcyclopropyl, 2,2-dichlorocyclopropyl, vinyl, 2-cyclopropylvinyl, 1-ethoxyvinyl, 2,2-dichlorovinyl, ethynyl, prop-1-ynyl, 2-bromoethynyl, 2-chloroethynyl, and 2-trimethylsilylethynyl.

Each R^{ba} is independently cyano, nitro, hydroxyl, C₃₋₆ cycloalkyl, C₁₋₄ alkoxy, C₁₋₄ alkylthio, C₁₋₄ alkylcarbonyl or C₁₋₄ alkoxycarbonyl, or two geminal R^{ba} together form an oxo or oximino group.

Each R^{bc} is independently halogen, cyano, C₁₋₄alkoxy, or C₁₋₄ alkoxycarbonyl. Preferably each R^{bc} is independently halogen or C₁₋₂ alkyl.

Each R^{bd} is independently halogen, cyano, C₃₋₆cycloalkyl, C₁₋₄alkylcarbonyl, or C₁₋₄ alkoxycarbonyl.

Each R^{be} is independently halogen, cyano, hydroxyl, C₁₋₄alkoxycarbonyl, or C₃₋₁₂ trialkylsilyl. Preferably each R^{be} is independently halogen or C₃₋₉ trialkylsilyl.

In preferred embodiments of the invention Z is Oₘ—(CH₂)ₙ—C(O)R^{cb}, wherein n is an integer of 0 or 1, m is an integer of 0 or 1, and both ₙ and ₘ have the same value, and wherein R^{cb} is hydroxyl, C₁₋₁₀ alkoxy, phenyl C₁₋₂ alkoxy or NH₂. More preferably still Z is selected from the group consisting of CO₂H, CO₂CH₃, CO₂CH₂CH₃, CO₂-i-propyl, CO₂-n-propyl, CO₂CH₂-i-propyl, CO₂CH₂-Phenyl, CONH₂, OCH₂CO₂H, OCH₂CO₂CH₃.

In alternative, preferred embodiments, Z is Oₘ—(CHR^{w})ₙ—C(O)R^{cb}, wherein
m is an integer of 0 or 1,
n is an integer of 0 or 1 and n≧m,
R^{w} is hydrogen or C₁₋₄ alkyl
R^{cb} is optionally substituted alkylthio, optionally substituted alkoxy or NH₂.

The compounds described below are illustrative of novel compounds of the invention. Table 1 below provides 402 compounds designated compound numbers 1-1 to 1-402 respectively, of formula (I) wherein A is chloro, and wherein the values of X, Y and Z are as given in Table 1.

TABLE 1

| Cmpd. | | | |
|---|---|---|---|
| No. | X | Y | Z |
| 1-1 | NH₂ | methyl | CO₂H |
| 1-2 | NH₂ | ethyl | CO₂H |
| 1-3 | NH₂ | n-propyl | CO₂H |
| 1-4 | NH₂ | isopropyl | CO₂H |
| 1-5 | NH₂ | difluoromethyl | CO₂H |
| 1-6 | NH₂ | trifluoromethyl | CO₂H |
| 1-7 | NH₂ | hydroxymethyl | CO₂H |
| 1-8 | NH₂ | methoxymethyl | CO₂H |
| 1-9 | NH₂ | 1-hydroxyethyl | CO₂H |
| 1-10 | NH₂ | 2-hydroxyethyl | CO₂H |
| 1-11 | NH₂ | 2-methoxyethyl | CO₂H |
| 1-12 | NH₂ | formyl | CO₂H |
| 1-13 | NH₂ | acetyl | CO₂H |
| 1-14 | NH₂ | hydroxyiminomethyl | CO₂H |
| 1-15 | NH₂ | methoxyiminomethyl | CO₂H |
| 1-16 | NH₂ | 1-(hydroxyimino)ethyl | CO₂H |
| 1-17 | NH₂ | 1-(methoxyimino)ethyl | CO₂H |
| 1-18 | NH₂ | cyclopropyl | CO₂H |
| 1-19 | NH₂ | 1-methylcyclopropyl | CO₂H |
| 1-20 | NH₂ | 2,2-dichlorocyclopropyl | CO₂H |
| 1-21 | NH₂ | vinyl | CO₂H |
| 1-22 | NH₂ | prop-1-enyl | CO₂H |
| 1-23 | NH₂ | prop-2-enyl | CO₂H |
| 1-24 | NH₂ | prop-1-en-2-yl | CO₂H |
| 1-25 | NH₂ | pent-1-enyl | CO₂H |
| 1-26 | NH₂ | 2-cyclopropylvinyl | CO₂H |
| 1-27 | NH₂ | 1-ethoxyvinyl | CO₂H |
| 1-28 | NH₂ | 1-methylprop-1-enyl | CO₂H |
| 1-29 | NH₂ | 2-methylprop-1-enyl | CO₂H |
| 1-30 | NH₂ | 1,2-dimethylprop-1-enyl | CO₂H |
| 1-31 | NH₂ | 2,2-dichlorovinyl | CO₂H |
| 1-32 | NH₂ | 3-methylbut-2-enyl | CO₂H |
| 1-33 | NH₂ | ethynyl | CO₂H |
| 1-34 | NH₂ | prop-1-ynyl | CO₂H |
| 1-35 | NH₂ | but-1-ynyl | CO₂H |
| 1-36 | NH₂ | 3-methylbut-1-ynyl | CO₂H |
| 1-37 | NH₂ | 3,3-dimethylbut-1-ynyl | CO₂H |
| 1-38 | NH₂ | 2-trimethylsilylethynyl | CO₂H |
| 1-39 | NH₂ | 2-chloroethynyl | CO₂H |
| 1-40 | NH₂ | 2-bromoethynyl | CO₂H |
| 1-41 | NH₂ | methyl | CO₂Me |
| 1-42 | NH₂ | ethyl | CO₂Me |
| 1-43 | NH₂ | n-propyl | CO₂Me |
| 1-44 | NH₂ | isopropyl | CO₂Me |
| 1-45 | NH₂ | difluoromethyl | CO₂Me |
| 1-46 | NH₂ | trifluoromethyl | CO₂Me |
| 1-47 | NH₂ | hydroxymethyl | CO₂Me |
| 1-48 | NH₂ | methoxymethyl | CO₂Me |
| 1-49 | NH₂ | 1-hydroxyethyl | CO₂Me |
| 1-50 | NH₂ | 2-hydroxyethyl | CO₂Me |
| 1-51 | NH₂ | 2-methoxyethyl | CO₂Me |
| 1-52 | NH₂ | formyl | CO₂Me |
| 1-53 | NH₂ | acetyl | CO₂Me |
| 1-54 | NH₂ | hydroxyiminomethyl | CO₂Me |
| 1-55 | NH₂ | methoxyiminomethyl | CO₂Me |
| 1-56 | NH₂ | 1-(hydroxyimino)ethyl | CO₂Me |
| 1-57 | NH₂ | 1-(methoxyimino)ethyl | CO₂Me |
| 1-58 | NH₂ | cyclopropyl | CO₂Me |
| 1-59 | NH₂ | 1-methylcyclopropyl | CO₂Me |
| 1-60 | NH₂ | 2,2-dichlorocyclopropyl | CO₂Me |
| 1-61 | NH₂ | vinyl | CO₂Me |
| 1-62 | NH₂ | prop-1-enyl | CO₂Me |
| 1-63 | NH₂ | prop-2-enyl | CO₂Me |
| 1-64 | NH₂ | prop-1-en-2-yl | CO₂Me |
| 1-65 | NH₂ | pent-1-enyl | CO₂Me |
| 1-66 | NH₂ | 2-cyclopropylvinyl | CO₂Me |
| 1-67 | NH₂ | 1-ethoxyvinyl | CO₂Me |
| 1-68 | NH₂ | 1-methylprop-1-enyl | CO₂Me |
| 1-69 | NH₂ | 2-methylprop-1-enyl | CO₂Me |
| 1-70 | NH₂ | 1,2-dimethylprop-1-enyl | CO₂Me |
| 1-71 | NH₂ | 2,2-dichlorovinyl | CO₂Me |
| 1-72 | NH₂ | 3-methylbut-2-enyl | CO₂Me |
| 1-73 | NH₂ | ethynyl | CO₂Me |
| 1-74 | NH₂ | prop-1-ynyl | CO₂Me |
| 1-75 | NH₂ | but-1-ynyl | CO₂Me |
| 1-76 | NH₂ | 3-methylbut-1-ynyl | CO₂Me |
| 1-77 | NH₂ | 3,3-dimethylbut-1-ynyl | CO₂Me |
| 1-78 | NH₂ | 2-trimethylsilylethynyl | CO₂Me |
| 1-79 | NH₂ | 2-chloroethynyl | CO₂Me |
| 1-80 | NH₂ | 2-bromoethynyl | CO₂Me |
| 1-81 | NH₂ | methyl | CO₂Et |
| 1-82 | NH₂ | ethyl | CO₂Et |
| 1-83 | NH₂ | n-propyl | CO₂Et |
| 1-84 | NH₂ | isopropyl | CO₂Et |
| 1-85 | NH₂ | difluoromethyl | CO₂Et |
| 1-86 | NH₂ | trifluoromethyl | CO₂Et |
| 1-87 | NH₂ | hydroxymethyl | CO₂Et |
| 1-88 | NH₂ | methoxymethyl | CO₂Et |
| 1-89 | NH₂ | 1-hydroxyethyl | CO₂Et |
| 1-90 | NH₂ | 2-hydroxyethyl | CO₂Et |
| 1-91 | NH₂ | 2-methoxyethyl | CO₂Et |
| 1-92 | NH₂ | formyl | CO₂Et |
| 1-93 | NH₂ | acetyl | CO₂Et |
| 1-94 | NH₂ | hydroxyiminomethyl | CO₂Et |
| 1-95 | NH₂ | methoxyiminomethyl | CO₂Et |
| 1-96 | NH₂ | 1-(hydroxyimino)ethyl | CO₂Et |
| 1-97 | NH₂ | 1-(methoxyimino)ethyl | CO₂Et |
| 1-98 | NH₂ | cyclopropyl | CO₂Et |
| 1-99 | NH₂ | 1-methylcyclopropyl | CO₂Et |
| 1-100 | NH₂ | 2,2-dichlorocyclopropyl | CO₂Et |
| 1-101 | NH₂ | vinyl | CO₂Et |
| 1-102 | NH₂ | prop-1-enyl | CO₂Et |
| 1-103 | NH₂ | prop-2-enyl | CO₂Et |
| 1-104 | NH₂ | prop-1-en-2-yl | CO₂Et |
| 1-105 | NH₂ | pent-1-enyl | CO₂Et |
| 1-106 | NH₂ | 2-cyclopropylvinyl | CO₂Et |
| 1-107 | NH₂ | 1-ethoxyvinyl | CO₂Et |
| 1-108 | NH₂ | 1-methylprop-1-enyl | CO₂Et |
| 1-109 | NH₂ | 2-methylprop-1-enyl | CO₂Et |
| 1-110 | NH₂ | 1,2-dimethylprop-1-enyl | CO₂Et |
| 1-111 | NH₂ | 2,2-dichlorovinyl | CO₂Et |
| 1-112 | NH₂ | 3-methylbut-2-enyl | CO₂Et |
| 1-113 | NH₂ | ethynyl | CO₂Et |
| 1-114 | NH₂ | prop-1-ynyl | CO₂Et |
| 1-115 | NH₂ | but-1-ynyl | CO₂Et |
| 1-116 | NH₂ | 3-methylbut-1-ynyl | CO₂Et |
| 1-117 | NH₂ | 3,3-dimethylbut-1-ynyl | CO₂Et |
| 1-118 | NH₂ | 2-trimethylsilylethynyl | CO₂Et |
| 1-119 | NH₂ | 2-chloroethynyl | CO₂Et |
| 1-120 | NH₂ | 2-bromoethynyl | CO₂Et |
| 1-121 | NHMe | methyl | CO₂H |
| 1-122 | NHMe | trifluoromethyl | CO₂H |
| 1-123 | NHMe | acetyl | CO₂H |
| 1-124 | NHMe | cyclopropyl | CO₂H |
| 1-125 | NHMe | vinyl | CO₂H |
| 1-126 | NHMe | prop-1-enyl | CO₂H |
| 1-127 | NHMe | prop-2-enyl | CO₂H |
| 1-128 | NHMe | ethynyl | CO₂H |
| 1-129 | NHMe | prop-1-ynyl | CO₂H |
| 1-130 | NHMe | 2-trimethylsilylethynyl | CO₂H |
| 1-131 | NMe₂ | methyl | CO₂H |
| 1-132 | NMe₂ | trifluoromethyl | CO₂H |
| 1-133 | NMe₂ | acetyl | CO₂H |
| 1-134 | NMe₂ | cyclopropyl | CO₂H |
| 1-135 | NMe₂ | vinyl | CO₂H |
| 1-136 | NMe₂ | prop-1-enyl | CO₂H |
| 1-137 | NMe₂ | prop-2-enyl | CO₂H |
| 1-138 | NMe₂ | ethynyl | CO₂H |
| 1-139 | NMe₂ | prop-1-ynyl | CO₂H |
| 1-140 | NMe₂ | 2-trimethylsilylethynyl | CO₂H |
| 1-141 | NHCOMe | methyl | CO₂H |
| 1-142 | NHCOMe | trifluoromethyl | CO₂H |
| 1-143 | NHCOMe | acetyl | CO₂H |
| 1-144 | NHCOMe | cyclopropyl | CO₂H |
| 1-145 | NHCOMe | vinyl | CO₂H |
| 1-146 | NHCOMe | prop-1-enyl | CO₂H |
| 1-147 | NHCOMe | prop-2-enyl | CO₂H |
| 1-148 | NHCOMe | ethynyl | CO₂H |
| 1-149 | NHCOMe | prop-1-ynyl | CO₂H |
| 1-150 | NHCOMe | 2-trimethylsilylethynyl | CO₂H |
| 1-151 | NHCO₂Me | methyl | CO₂H |
| 1-152 | NHCO₂Me | trifluoromethyl | CO₂H |
| 1-153 | NHCO₂Me | acetyl | CO₂H |
| 1-154 | NHCO₂Me | cyclopropyl | CO₂H |
| 1-155 | NHCO₂Me | vinyl | CO₂H |
| 1-156 | NHCO₂Me | prop-1-enyl | CO₂H |
| 1-157 | NHCO₂Me | prop-2-enyl | CO₂H |
| 1-158 | NHCO₂Me | ethynyl | CO₂H |
| 1-159 | NHCO₂Me | prop-1-ynyl | CO₂H |
| 1-160 | NHCO₂Me | 2-trimethylsilylethynyl | CO₂H |
| 1-161 | NHSO₂Me | methyl | CO₂H |
| 1-162 | NHSO₂Me | trifluoromethyl | CO₂H |
| 1-163 | NHSO₂Me | acetyl | CO₂H |
| 1-164 | NHSO₂Me | cyclopropyl | CO₂H |
| 1-165 | NHSO₂Me | vinyl | CO₂H |
| 1-166 | NHSO₂Me | prop-1-enyl | CO₂H |
| 1-167 | NHSO₂Me | prop-2-enyl | CO₂H |
| 1-168 | NHSO₂Me | ethynyl | CO₂H |
| 1-169 | NHSO₂Me | prop-1-ynyl | CO₂H |
| 1-170 | NHSO₂Me | 2-trimethylsilylethynyl | CO₂H |
| 1-171 | NMeCOMe | methyl | CO₂H |
| 1-172 | NMeCOMe | trifluoromethyl | CO₂H |
| 1-173 | NMeCOMe | acetyl | CO₂H |
| 1-174 | NMeCOMe | cyclopropyl | CO₂H |
| 1-175 | NMeCOMe | vinyl | CO₂H |
| 1-176 | NMeCOMe | prop-1-enyl | CO₂H |
| 1-177 | NMeCOMe | prop-2-enyl | CO₂H |
| 1-178 | NMeCOMe | ethynyl | CO₂H |
| 1-179 | NMeCOMe | prop-1-ynyl | CO₂H |
| 1-180 | NMeCOMe | 2-trimethylsilylethynyl | CO₂H |
| 1-181 | NMeCO₂Me | methyl | CO₂H |
| 1-182 | NMeCO₂Me | trifluoromethyl | CO₂H |
| 1-183 | NMeCO₂Me | acetyl | CO₂H |
| 1-184 | NMeCO₂Me | cyclopropyl | CO₂H |
| 1-185 | NMeCO₂Me | vinyl | CO₂H |
| 1-186 | NMeCO₂Me | prop-1-enyl | CO₂H |
| 1-187 | NMeCO₂Me | prop-2-enyl | CO₂H |
| 1-188 | NMeCO₂Me | ethynyl | CO₂H |
| 1-189 | NMeCO₂Me | prop-1-ynyl | CO₂H |
| 1-190 | NMeCO₂Me | 2-trimethylsilylethynyl | CO₂H |
| 1-191 | NMeSO₂Me | methyl | CO₂H |
| 1-192 | NMeSO₂Me | trifluoromethyl | CO₂H |
| 1-193 | NMeSO₂Me | acetyl | CO₂H |
| 1-194 | NMeSO₂Me | cyclopropyl | CO₂H |
| 1-195 | NMeSO₂Me | vinyl | CO₂H |
| 1-196 | NMeSO₂Me | prop-1-enyl | CO₂H |
| 1-197 | NMeSO₂Me | prop-2-enyl | CO₂H |
| 1-198 | NMeSO₂Me | ethynyl | CO₂H |
| 1-199 | NMeSO₂Me | prop-1-ynyl | CO₂H |
| 1-200 | NMeSO₂Me | 2-trimethylsilylethynyl | CO₂H |
| 1-201 | NHⁱPr | methyl | CO₂H |
| 1-202 | NHⁱPr | trifluoromethyl | CO₂H |
| 1-203 | NHⁱPr | acetyl | CO₂H |
| 1-204 | NHⁱPr | cyclopropyl | CO₂H |
| 1-205 | NHⁱPr | vinyl | CO₂H |
| 1-206 | NHⁱPr | prop-1-enyl | CO₂H |
| 1-207 | NHⁱPr | prop-2-enyl | CO₂H |
| 1-208 | NHⁱPr | ethynyl | CO₂H |
| 1-209 | NHⁱPr | prop-1-ynyl | CO₂H |
| 1-210 | NHⁱPr | 2-trimethylsilylethynyl | CO₂H |
| 1-211 | NHMe | methyl | CO₂Me |
| 1-212 | NHMe | trifluoromethyl | CO₂Me |
| 1-213 | NHMe | acetyl | CO₂Me |
| 1-214 | NHMe | cyclopropyl | CO₂Me |
| 1-215 | NHMe | vinyl | CO₂Me |
| 1-216 | NHMe | prop-1-enyl | CO₂Me |
| 1-217 | NHMe | prop-2-enyl | CO₂Me |
| 1-218 | NHMe | ethynyl | CO₂Me |
| 1-219 | NHMe | prop-1-ynyl | CO₂Me |
| 1-220 | NHMe | 2-trimethylsilylethynyl | CO₂Me |
| 1-221 | NMe₂ | methyl | CO₂Me |
| 1-222 | NMe₂ | trifluoromethyl | CO₂Me |
| 1-223 | NMe₂ | acetyl | CO₂Me |
| 1-224 | NMe₂ | cyclopropyl | CO₂Me |
| 1-225 | NMe₂ | vinyl | CO₂Me |
| 1-226 | NMe₂ | prop-1-enyl | CO₂Me |
| 1-227 | NMe₂ | prop-2-enyl | CO₂Me |
| 1-228 | NMe₂ | ethynyl | CO₂Me |
| 1-229 | NMe₂ | prop-1-ynyl | CO₂Me |
| 1-230 | NMe₂ | 2-trimethylsilylethynyl | CO₂Me |
| 1-231 | NHCOMe | methyl | CO₂Me |
| 1-232 | NHCOMe | trifluoromethyl | CO₂Me |
| 1-233 | NHCOMe | acetyl | CO₂Me |
| 1-234 | NHCOMe | cyclopropyl | CO₂Me |
| 1-235 | NHCOMe | vinyl | CO₂Me |
| 1-236 | NHCOMe | prop-1-enyl | CO₂Me |
| 1-237 | NHCOMe | prop-2-enyl | CO₂Me |
| 1-238 | NHCOMe | ethynyl | CO₂Me |
| 1-239 | NHCOMe | prop-1-ynyl | CO₂Me |
| 1-240 | NHCOMe | 2-trimethylsilylethynyl | CO₂Me |
| 1-241 | NHCO₂Me | methyl | CO₂Me |
| 1-242 | NHCO₂Me | trifluoromethyl | CO₂Me |
| 1-243 | NHCO₂Me | acetyl | CO₂Me |
| 1-244 | NHCO₂Me | cyclopropyl | CO₂Me |
| 1-245 | NHCO₂Me | vinyl | CO₂Me |
| 1-246 | NHCO₂Me | prop-1-enyl | CO₂Me |
| 1-247 | NHCO₂Me | prop-2-enyl | CO₂Me |
| 1-248 | NHCO₂Me | ethynyl | CO₂Me |
| 1-249 | NHCO₂Me | prop-1-ynyl | CO₂Me |
| 1-250 | NHCO₂Me | 2-trimethylsilylethynyl | CO₂Me |
| 1-251 | NHSO₂Me | methyl | CO₂Me |
| 1-252 | NHSO₂Me | trifluoromethyl | CO₂Me |
| 1-253 | NHSO₂Me | acetyl | CO₂Me |
| 1-254 | NHSO₂Me | cyclopropyl | CO₂Me |
| 1-255 | NHSO₂Me | vinyl | CO₂Me |
| 1-256 | NHSO₂Me | prop-1-enyl | CO₂Me |
| 1-257 | NHSO₂Me | prop-2-enyl | CO₂Me |
| 1-258 | NHSO₂Me | ethynyl | CO₂Me |
| 1-259 | NHSO₂Me | prop-1-ynyl | CO₂Me |
| 1-260 | NHSO₂Me | 2-trimethylsilylethynyl | CO₂Me |
| 1-261 | NMeCOMe | methyl | CO₂Me |
| 1-262 | NMeCOMe | trifluoromethyl | CO₂Me |
| 1-263 | NMeCOMe | acetyl | CO₂Me |
| 1-264 | NMeCOMe | cyclopropyl | CO₂Me |
| 1-265 | NMeCOMe | vinyl | CO₂Me |
| 1-266 | NMeCOMe | prop-1-enyl | CO₂Me |
| 1-267 | NMeCOMe | prop-2-enyl | CO₂Me |
| 1-268 | NMeCOMe | ethynyl | CO₂Me |
| 1-269 | NMeCOMe | prop-1-ynyl | CO₂Me |
| 1-270 | NMeCOMe | 2-trimethylsilylethynyl | CO₂Me |
| 1-271 | NMeCO₂Me | methyl | CO₂Me |
| 1-272 | NMeCO₂Me | trifluoromethyl | CO₂Me |
| 1-273 | NMeCO₂Me | acetyl | CO₂Me |
| 1-274 | NMeCO₂Me | cyclopropyl | CO₂Me |
| 1-275 | NMeCO₂Me | vinyl | CO₂Me |
| 1-276 | NMeCO₂Me | prop-1-enyl | CO₂Me |
| 1-277 | NMeCO₂Me | prop-2-enyl | CO₂Me |
| 1-278 | NMeCO₂Me | ethynyl | CO₂Me |
| 1-279 | NMeCO₂Me | prop-1-ynyl | CO₂Me |
| 1-280 | NMeCO₂Me | 2-trimethylsilylethynyl | CO₂Me |
| 1-281 | NMeSO₂Me | methyl | CO₂Me |
| 1-282 | NMeSO₂Me | trifluoromethyl | CO₂Me |
| 1-283 | NMeSO₂Me | acetyl | CO₂Me |
| 1-284 | NMeSO₂Me | cyclopropyl | CO₂Me |
| 1-285 | NMeSO₂Me | vinyl | CO₂Me |
| 1-286 | NMeSO₂Me | prop-1-enyl | CO₂Me |
| 1-287 | NMeSO₂Me | prop-2-enyl | CO₂Me |
| 1-288 | NMeSO₂Me | ethynyl | CO₂Me |
| 1-289 | NMeSO₂Me | prop-1-ynyl | CO₂Me |
| 1-290 | NMeSO₂Me | 2-trimethylsilylethynyl | CO₂Me |
| 1-291 | NHⁱPr | methyl | CO₂Me |
| 1-292 | NHⁱPr | trifluoromethyl | CO₂Me |
| 1-293 | NHⁱPr | acetyl | CO₂Me |
| 1-294 | NHⁱPr | cyclopropyl | CO₂Me |
| 1-295 | NHⁱPr | vinyl | CO₂Me |
| 1-296 | NHⁱPr | prop-1-enyl | CO₂Me |
| 1-297 | NHⁱPr | prop-2-enyl | CO₂Me |
| 1-298 | NHⁱPr | ethynyl | CO₂Me |
| 1-299 | NHⁱPr | prop-1-ynyl | CO₂Me |
| 1-300 | NHⁱPr | 2-trimethylsilylethynyl | CO₂Me |
| 1-301 | NMe₂ | methyl | CO₂Et |
| 1-302 | NH₂ | methyl | CO₂ⁱPr |
| 1-303 | NH₂ | trifluoromethyl | CO₂ⁱPr |
| 1-304 | NH₂ | acetyl | CO₂ⁱPr |
| 1-305 | NH₂ | cyclopropyl | CO₂ⁱPr |
| 1-306 | NH₂ | vinyl | CO₂ⁱPr |
| 1-307 | NH₂ | prop-1-enyl | CO₂ⁱPr |
| 1-308 | NH₂ | prop-2-enyl | CO₂ⁱPr |
| 1-309 | NH₂ | ethynyl | CO₂ⁱPr |
| 1-310 | NH₂ | prop-1-ynyl | CO₂ⁱPr |
| 1-311 | NH₂ | 2-trimethylsilylethynyl | CO₂ⁱPr |
| 1-312 | NH₂ | methyl | CO₂ⁿPr |
| 1-313 | NH₂ | trifluoromethyl | CO₂ⁿPr |
| 1-314 | NH₂ | acetyl | CO₂ⁿPr |
| 1-315 | NH₂ | cyclopropyl | CO₂ⁿPr |
| 1-316 | NH₂ | vinyl | CO₂ⁿPr |
| 1-317 | NH₂ | prop-1-enyl | CO₂ⁿPr |
| 1-318 | NH₂ | prop-2-enyl | CO₂ⁿPr |
| 1-319 | NH₂ | ethynyl | CO₂ⁿPr |
| 1-320 | NH₂ | prop-1-ynyl | CO₂ⁿPr |
| 1-321 | NH₂ | 2-trimethylsilylethynyl | CO₂ⁿPr |
| 1-322 | NH₂ | methyl | CO₂CH₂ⁱPr |
| 1-323 | NH₂ | trifluoromethyl | CO₂CH₂ⁱPr |
| 1-324 | NH₂ | acetyl | CO₂CH₂ⁱPr |
| 1-325 | NH₂ | cyclopropyl | CO₂CH₂ⁱPr |
| 1-326 | NH₂ | vinyl | CO₂CH₂ⁱPr |
| 1-327 | NH₂ | prop-1-enyl | CO₂CH₂ⁱPr |
| 1-328 | NH₂ | prop-2-enyl | CO₂CH₂ⁱPr |
| 1-329 | NH₂ | ethynyl | CO₂CH₂ⁱPr |
| 1-330 | NH₂ | prop-1-ynyl | CO₂CH₂ⁱPr |
| 1-331 | NH₂ | 2-trimethylsilylethynyl | CO₂CH₂ⁱPr |
| 1-332 | NH₂ | methyl | CO₂CH₂Ph |
| 1-333 | NH₂ | trifluoromethyl | CO₂CH₂Ph |
| 1-334 | NH₂ | acetyl | CO₂CH₂Ph |
| 1-335 | NH₂ | cyclopropyl | CO₂CH₂Ph |
| 1-336 | NH₂ | vinyl | CO₂CH₂Ph |
| 1-337 | NH₂ | prop-1-enyl | CO₂CH₂Ph |
| 1-338 | NH₂ | prop-2-enyl | CO₂CH₂Ph |
| 1-339 | NH₂ | ethynyl | CO₂CH₂Ph |
| 1-340 | NH₂ | prop-1-ynyl | CO₂CH₂Ph |
| 1-341 | NH₂ | 2-trimethylsilylethynyl | CO₂CH₂Ph |
| 1-342 | NH₂ | methyl | CONH₂ |
| 1-343 | NH₂ | trifluoromethyl | CONH₂ |
| 1-344 | NH₂ | acetyl | CONH₂ |
| 1-345 | NH₂ | cyclopropyl | CONH₂ |
| 1-346 | NH₂ | vinyl | CONH₂ |
| 1-347 | NH₂ | prop-1-enyl | CONH₂ |
| 1-348 | NH₂ | prop-2-enyl | CONH₂ |
| 1-349 | NH₂ | ethynyl | CONH₂ |
| 1-350 | NH₂ | prop-1-ynyl | CONH₂ |
| 1-351 | NH₂ | 2-trimethylsilylethynyl | CONH₂ |
| 1-352 | NH₂ | methyl | OCH₂CO₂H |
| 1-353 | NH₂ | trifluoromethyl | OCH₂CO₂H |
| 1-354 | NH₂ | acetyl | OCH₂CO₂H |
| 1-355 | NH₂ | cyclopropyl | OCH₂CO₂H |
| 1-356 | NH₂ | vinyl | OCH₂CO₂H |
| 1-357 | NH₂ | prop-1-enyl | OCH₂CO₂H |
| 1-358 | NH₂ | prop-2-enyl | OCH₂CO₂H |
| 1-359 | NH₂ | ethynyl | OCH₂CO₂H |
| 1-360 | NH₂ | prop-1-ynyl | OCH₂CO₂H |
| 1-361 | NH₂ | 2-trimethylsilylethynyl | OCH₂CO₂H |
| 1-362 | NH₂ | methyl | OCH₂CO₂Me |
| 1-363 | NH₂ | trifluoromethyl | OCH₂CO₂Me |
| 1-364 | NH₂ | acetyl | OCH₂CO₂Me |
| 1-365 | NH₂ | cyclopropyl | OCH₂CO₂Me |
| 1-366 | NH₂ | vinyl | OCH₂CO₂Me |
| 1-367 | NH₂ | prop-1-enyl | OCH₂CO₂Me |
| 1-368 | NH₂ | prop-2-enyl | OCH₂CO₂Me |
| 1-369 | NH₂ | ethynyl | OCH₂CO₂Me |
| 1-370 | NH₂ | prop-1-ynyl | OCH₂CO₂Me |
| 1-371 | NH₂ | 2-trimethylsilylethynyl | OCH₂CO₂Me |
| 1-372 | NH₂ | 1-hydroxyethyl | CH₂OH |
| 1-373 | Cl | methyl | CO₂H |
| 1-374 | Cl | methyl | CO₂Me |
| 1-375 | Cl | methyl | OCH₂CO₂H |
| 1-376 | Cl | methyl | OCH₂CO₂Me |
| 1-377 | OMe | methyl | CO₂H |
| 1-378 | OMe | methyl | CO₂Me |
| 1-379 | OMe | methyl | OCH₂CO₂H |
| 1-380 | OMe | methyl | OCH₂CO₂Me |
| 1-381 | OCH₂CO₂Me | methyl | CO₂H |
| 1-382 | OCH₂CO₂Me | methyl | CO₂Me |
| 1-383 | OCH₂CO₂Me | methyl | OCH₂CO₂H |
| 1-384 | OCH₂CO₂Me | methyl | OCH₂CO₂Me |
| 1-385 | N₃ | methyl | CO₂H |
| 1-386 | N₃ | methyl | CO₂Me |
| 1-387 | N₃ | methyl | OCH₂CO₂H |
| 1-388 | N₃ | methyl | OCH₂CO₂Me |
| 1-389 | OCH₂CO₂^{t}Bu | methyl | OCH₂CO₂^{t}Bu |
| 1-390 | Cl | methyl | CO₂Et |
| 1-391 | NH₂ | vinyl | CO₂ⁿBu |
| 1-392 | NH₂ | vinyl | CO₂ⁿoctyl |
| 1-393 | NH₂ | vinyl | CO₂(2-ethylhexyl) |
| 1-394 | NH₂ | vinyl | CO₂CH(Me)Ph |
| 1-395 | NH₂ | vinyl | CO₂prop-2-enyl |
| 1-396 | NH₂ | vinyl | CO₂(CH₂)₂OEt |
| 1-397 | NH₂ | vinyl | CO₂(CH₂)₂OⁿBu |
| 1-398 | OMe | vinyl | CO₂Me |
| 1-399 | N₃ | vinyl | CO₂Me |
| 1-400 | SMe | vinyl | CO₂Me |
| 1-401 | SOMe | vinyl | CO₂Me |
| 1-402 | SO₂Me | vinyl | CO₂Me |

402 compounds of formula (I), wherein A is cyclopropyl, and the values of X, Y and Z are as given in Table 1 for compounds 1-1 to 1-402, are designated as compound Nos. 2-1 to 2-402, respectively.

402 compounds of formula (I), wherein A is 2-methylcyclopropyl, and the values of X, Y and Z are as given in Table 1 for compounds 1-1 to 1-402, are designated as compound Nos. 3-1 to 3-402, respectively.

402 compounds of formula (I), wherein A is isopropyl, and the values of X, Y and Z are as given in Table 1 for compounds 1-1 to 1-402, are designated as compound Nos. 4-1 to 4-402, respectively.

402 compounds of formula (I), wherein A is 4-chlorophenyl, and the values of X, Y and Z are as given in Table 1 for compounds 1-1 to 1-402, are designated as compound Nos. 5-1 to 5-402, respectively.

402 compounds of formula (I), wherein A is 4-bromophenyl, and the values of X, Y and Z are as given in Table 1 for compounds 1-1 to 1-402, are designated as compound Nos. 6-1 to 6-402, respectively.

402 compounds of formula (I), wherein A is 4-iodophenyl, and the values of X, Y and Z are as given in Table 1 for compounds 1-1 to 1-402, are designated as compound Nos. 7-1 to 7-402, respectively.

402 compounds of formula (I), wherein A is 2,4-dichlorophenyl, and the values of X, Y and Z are as given in Table 1 for compounds 1-1 to 1-402, are designated as compound Nos. 8-1 to 8-402, respectively.

402 compounds of formula (I), wherein A is 3,4-dichlorophenyl, and the values of X, Y and Z are as given in Table 1 for compounds 1-1 to 1-402, are designated as compound Nos. 9-1 to 9-402, respectively.

402 compounds of formula (I), wherein A is 4-chloro-2-methylphenyl, and the values of X, Y and Z are as given in Table 1 for compounds 1-1 to 1-402, are designated as compound Nos. 10-1 to 10-402, respectively.

402 compounds of formula (I), wherein A is 4-chloro-2-methoxyphenyl, and the values of X, Y and Z are as given in Table 1 for compounds 1-1 to 1-402, are designated as compound Nos. 11-1 to 11-402, respectively.

402 compounds of formula (I), wherein A is 4-chloro-2,6-difluorophenyl, and the values of X, Y and Z are as given in Table 1 for compounds 1-1 to 1-402, are designated as compound Nos. 12-1 to 12-402, respectively.

402 compounds of formula (I), wherein A is 4-chloro-3-methylphenyl, and the values of X, Y and Z are as given in Table 1 for compounds 1-1 to 1-402, are designated as compound Nos. 13-1 to 13-402, respectively.

402 compounds of formula (I), wherein A is 4-chloro-3-trifluoromethylphenyl, and the values of X, Y and Z are as given in Table 1 for compounds 1-1 to 1-402, are designated as compound Nos. 14-1 to 14-402, respectively.

402 compounds of formula (I), wherein A is 4-chloro-2-fluorophenyl, and the values of X, Y and Z are as given in Table 1 for compounds 1-1 to 1-402, are designated as compound Nos. 15-1 to 15-402, respectively.

402 compounds of formula (I), wherein A is 4-chloro-3-nitrophenyl, and the values of X, Y and Z are as given in Table 1 for compounds 1-1 to 1-402, are designated as compound Nos. 16-1 to 16-402, respectively.

402 compounds of formula (I), wherein A is 4-chloro-2-trifluoromethylphenyl, and the values of X, Y and Z are as given in Table 1 for compounds 1-1 to 1-402, are designated as compound Nos. 17-1 to 17-402, respectively.

402 compounds of formula (I), wherein A is 4-chloro-3-cyanophenyl, and the values of X, Y and Z are as given in Table 1 for compounds 1-1 to 1-402, are designated as compound Nos. 18-1 to 18-402, respectively.

402 compounds of formula (I), wherein A is 4-chloro-3-fluorophenyl, and the values of X, Y and Z are as given in Table 1 for compounds 1-1 to 1-402, are designated as compound Nos. 19-1 to 19-402, respectively.

402 compounds of formula (I), wherein A is 2,4,6-trichlorophenyl, and the values of X, Y and Z are as given in Table 1 for compounds 1-1 to 1-402, are designated as compound Nos. 20-1 to 20-402, respectively.

402 compounds of formula (I), wherein A is 2,4-dichloro-3-fluorophenyl, and the values of X, Y and Z are as given in Table 1 for compounds 1-1 to 1-402, are designated as compound Nos. 21-1 to 21-402, respectively.

402 compounds of formula (I), wherein A is 4-chloro-2,3-difluorophenyl, and the values of X, Y and Z are as given in Table 1 for compounds 1-1 to 1-402, are designated as compound Nos. 22-1 to 22-402, respectively.

402 compounds of formula (I), wherein A is 3,4-dichloro-2-fluorophenyl, and the values of X, Y and Z are as given in Table 1 for compounds 1-1 to 1-402, are designated as compound Nos. 23-1 to 23-402, respectively.

402 compounds of formula (I), wherein A is 2,4-dichloro-6-fluorophenyl, and the values of X, Y and Z are as given in Table 1 for compounds 1-1 to 1-402, are designated as compound Nos. 24-1 to 24-402, respectively.

402 compounds of formula (I), wherein A is 2,4,5-trichlorophenyl, and the values of X, Y and Z are as given in Table 1 for compounds 1-1 to 1-402, are designated as compound Nos. 25-1 to 25-402, respectively.

402 compounds of formula (I), wherein A is 4-chloro-3-methoxyphenyl, and the values of X, Y and Z are as given in Table 1 for compounds 1-1 to 1-402, are designated as compound Nos. 26-1 to 26-402, respectively.

402 compounds of formula (I), wherein A is 4-chloro-2-fluoro-3-methoxyphenyl, and the values of X, Y and Z are as given in Table 1 for compounds 1-1 to 1-402, are designated as compound Nos. 27-1 to 27-402, respectively.

402 compounds of formula (I), wherein A is 4-chloro-2-fluoro-3-trifluoromethylphenyl, and the values of X, Y and Z are as given in Table 1 for compounds 1-1 to 1-402, are designated as compound Nos. 28-1 to 28-402, respectively.

402 compounds of formula (I), wherein A is 2,3,4-trichlorophenyl, and the values of X, Y and Z are as given in Table 1 for compounds 1-1 to 1-402, are designated as compound Nos. 29-1 to 29-402, respectively.

402 compounds of formula (I), wherein A is 4-chloro-3,5-difluorophenyl, and the values of X, Y and Z are as given in Table 1 for compounds 1-1 to 1-402, are designated as compound Nos. 30-1 to 30-402, respectively.

402 compounds of formula (I), wherein A is 4-trifluoromethylphenyl, and the values of X, Y and Z are as given in Table 1 for compounds 1-1 to 1-402, are designated as compound Nos. 31-1 to 31-402, respectively.

402 compounds of formula (I), wherein A is 2,4-bis(trifluoromethyl)phenyl, and the values of X, Y and Z are as given in Table 1 for compounds 1-1 to 1-402, are designated as compound Nos. 32-1 to 32-402, respectively.

402 compounds of formula (I), wherein A is 4-chloro-3-dimethylamino-2-fluorophenyl, and the values of X, Y and Z are as given in Table 1 for compounds 1-1 to 1-402, are designated as compound Nos. 33-1 to 33-402, respectively.

402 compounds of formula (I), wherein A is 3-chloro-4-trifluoromethylphenyl, and the values of X, Y and Z are as given in Table 1 for compounds 1-1 to 1-402, are designated as compound Nos. 34-1 to 34-402, respectively.

402 compounds of formula (I), wherein A is 2-chloro-4-trifluoromethylphenyl, and the values of X, Y and Z are as given in Table 1 for compounds 1-1 to 1-402, are designated as compound Nos. 35-1 to 35-402, respectively.

402 compounds of formula (I), wherein A is 4-methylphenyl, and the values of X, Y and Z are as given in Table 1 for compounds 1-1 to 1-402, are designated as compound Nos. 36-1 to 36-402, respectively.

402 compounds of formula (I), wherein A is 2-chloro-4-methylphenyl, and the values of X, Y and Z are as given in Table 1 for compounds 1-1 to 1-402, are designated as compound Nos. 37-1 to 37-402, respectively.

402 compounds of formula (I), wherein A is 3-chloro-4-methylphenyl, and the values of X, Y and Z are as given in Table 1 for compounds 1-1 to 1-402, are designated as compound Nos. 38-1 to 38-402, respectively.

402 compounds of formula (I), wherein A is 3-fluoro-4-methylphenyl, and the values of X, Y and Z are as given in Table 1 for compounds 1-1 to 1-402, are designated as compound Nos. 39-1 to 39-402, respectively.

402 compounds of formula (I), wherein A is 2,4,5-trimethylphenyl, and the values of X, Y and Z are as given in Table 1 for compounds 1-1 to 1-402, are designated as compound Nos. 40-1 to 40-402, respectively.

402 compounds of formula (I), wherein A is 4-fluorophenyl, and the values of X, Y and Z are as given in Table 1 for compounds 1-1 to 1-402, are designated as compound Nos. 41-1 to 41-402, respectively.

402 compounds of formula (I), wherein A is 4-fluoro-3-methoxyphenyl, and the values of X, Y and Z are as given in Table 1 for compounds 1-1 to 1-402, are designated as compound Nos. 42-1 to 42-402, respectively.

402 compounds of formula (I), wherein A is 4-fluoro-3-trifluoromethylphenyl, and the values of X, Y and Z are as given in Table 1 for compounds 1-1 to 1-402, are designated as compound Nos. 43-1 to 43-402, respectively.

402 compounds of formula (I), wherein A is 3-chloro-4-fluorophenyl, and the values of X, Y and Z are as given in Table 1 for compounds 1-1 to 1-402, are designated as compound Nos. 44-1 to 44-402, respectively.

402 compounds of formula (I), wherein A is 4-fluoro-3-methylphenyl, and the values of X, Y and Z are as given in Table 1 for compounds 1-1 to 1-402, are designated as compound Nos. 45-1 to 45-402, respectively.

402 compounds of formula (I), wherein A is 2,3,4-trifluorophenyl, and the values of X, Y and Z are as given in Table 1 for compounds 1-1 to 1-402, are designated as compound Nos. 46-1 to 46-402, respectively.

402 compounds of formula (I), wherein A is 4-trifluoromethoxyphenyl, and the values of X, Y and Z are as given in Table 1 for compounds 1-1 to 1-402, are designated as compound Nos. 47-1 to 47-402, respectively.

402 compounds of formula (I), wherein A is 2-fluoro-3-methoxyphenyl, and the values of X, Y and Z are as given in Table 1 for compounds 1-1 to 1-402, are designated as compound Nos. 48-1 to 48-402, respectively.

402 compounds of formula (I), wherein A is 4-methoxyphenyl, and the values of X, Y and Z are as given in Table 1 for compounds 1-1 to 1-402, are designated as compound Nos. 49-1 to 49-402, respectively.

402 compounds of formula (I), wherein A is 4-methoxy-2-methylphenyl, and the values of X, Y and Z are as given in Table 1 for compounds 1-1 to 1-402, are designated as compound Nos. 50-1 to 50-402, respectively.

402 compounds of formula (I), wherein A is 2,4-dimethoxyphenyl, and the values of X, Y and Z are as given in Table 1 for compounds 1-1 to 1-402, are designated as compound Nos. 51-1 to 51-402, respectively.

402 compounds of formula (I), wherein A is 3-fluoro-4-methoxyphenyl, and the values of X, Y and Z are as given in Table 1 for compounds 1-1 to 1-402, are designated as compound Nos. 52-1 to 52-402, respectively.

402 compounds of formula (I), wherein A is 3-chloro-4-methoxyphenyl, and the values of X, Y and Z are as given in Table 1 for compounds 1-1 to 1-402, are designated as compound Nos. 53-1 to 53-402, respectively.

402 compounds of formula (I), wherein A is 3,4-dimethylphenyl, and the values of X, Y and Z are as given in Table 1 for compounds 1-1 to 1-402, are designated as compound Nos. 54-1 to 54-402, respectively.

402 compounds of formula (I), wherein A is 4-methyl-3-nitrophenyl, and the values of X, Y and Z are as given in Table 1 for compounds 1-1 to 1-402, are designated as compound Nos. 55-1 to 55-402, respectively.

402 compounds of formula (I), wherein A is 2-fluoro-4-methylphenyl, and the values of X, Y and Z are as given in Table 1 for compounds 1-1 to 1-402, are designated as compound Nos. 56-1 to 56-402, respectively.

402 compounds of formula (I), wherein A is 2-fluoro-4-methoxyphenyl, and the values of X, Y and Z are as given in Table 1 for compounds 1-1 to 1-402, are designated as compound Nos. 57-1 to 57-402, respectively.

402 compounds of formula (I), wherein A is 2,3,4-trimethoxyphenyl, and the values of X, Y and Z are as given in Table 1 for compounds 1-1 to 1-402, are designated as compound Nos. 58-1 to 58-402, respectively.

402 compounds of formula (I), wherein A is 3,4-dimethoxyphenyl, and the values of X, Y and Z are as given in Table 1 for compounds 1-1 to 1-402, are designated as compound Nos. 59-1 to 59-402, respectively.

402 compounds of formula (I), wherein A is 2-fluoro-3-methoxy-4-methylphenyl, and the values of X, Y and Z are as given in Table 1 for compounds 1-1 to 1-402, are designated as compound Nos. 60-1 to 60-402, respectively.

402 compounds of formula (I), wherein A is 3-chloro-5-fluorophenyl, and the values of X, Y and Z are as given in Table 1 for compounds 1-1 to 1-402, are designated as compound Nos. 61-1 to 61-402, respectively.

402 compounds of formula (I), wherein A is methylthio, and the values of X, Y and Z are as given in Table 1 for compounds 1-1 to 1-402, are designated as compound Nos. 62-1 to 62-402, respectively.

402 compounds of formula (I), wherein A is 3,4,5-trifluorophenyl, and the values of X, Y and Z are as given in Table 1 for compounds 1-1 to 1-402, are designated as compound Nos. 63-1 to 63-402, respectively.

402 compounds of formula (I), wherein A is 3-dimethylamino-4-ethenyl-2-fluorophenyl, and the values of X, Y and Z are as given in Table 1 for compounds 1-1 to 1-402, are designated as compound Nos. 64-1 to 64-402, respectively.

402 compounds of formula (I), wherein A is 4-acetyl-2-fluoro-3-methoxyphenyl, and the values of X, Y and Z are as given in Table 1 for compounds 1-1 to 1-402, are designated as compound Nos. 65-1 to 65-402, respectively.

402 compounds of formula (I), wherein A is 4-(1-ethoxyethenyl)-2-fluoro-3-methoxyphenyl, and the values of X, Y and Z are as given in Table 1 for compounds 1-1 to 1-402, are designated as compound Nos. 66-1 to 66-402, respectively.

402 compounds of formula (I), wherein A is 3,4-difluorophenyl, and the values of X, Y and Z are as given in Table 1 for compounds 1-1 to 1-402, are designated as compound Nos. 67-1 to 67-402, respectively.

402 compounds of formula (I), wherein A is 4-cyanophenyl, and the values of X, Y and Z are as given in Table 1 for compounds 1-1 to 1-402, are designated as compound Nos. 68-1 to 68-402, respectively.

402 compounds of formula (I), wherein A is 4-methoxycarbonylphenyl, and the values of X, Y and Z are as given in Table 1 for compounds 1-1 to 1-402, are designated as compound Nos. 69-1 to 69-402, respectively.

402 compounds of formula (I), wherein A is 3,4-methylenedioxyphenyl, and the values of X, Y and Z are as given in Table 1 for compounds 1-1 to 1-402, are designated as compound Nos. 70-1 to 70-402, respectively.

402 compounds of formula (I), wherein A is 3,4-ethylenedioxyphenyl, and the values of X, Y and Z are as given in Table 1 for compounds 1-1 to 1-402, are designated as compound Nos. 71-1 to 71-402, respectively.

402 compounds of formula (I), wherein A is 4-methylthiophenyl, and the values of X, Y and Z are as given in Table 1 for compounds 1-1 to 1-402, are designated as compound Nos. 72-1 to 72-402, respectively.

402 compounds of formula (I), wherein A is 4-naphthyl, and the values of X, Y and Z are as given in Table 1 for compounds 1-1 to 1-402, are designated as compound Nos. 73-1 to 73-402, respectively.

General methods for the production of compounds of formula (I) are described below. Unless otherwise stated in the text, the substitutents A, X, Y and Z are as defined hereinbefore. The abbreviation LG as used herein refers to any suitable leaving group, and includes halogen, sulphonate, and sulphone groups. The groups R as used herein are, independently of each other, alkyl or substituted alkyl groups, preferably C1-C12 alkyl groups. The groups R′ may, independently of each other, take a range of values depending on the particular structure of the molecule in which they are present; the skilled man will recognise what values are applicable in each case, particularly in view of the definition of compounds of formula (I) as described hereinbefore.

Compounds of formula (I) may be prepared from compounds of formula (A), where LG represents a suitable leaving group (reaction scheme 1).

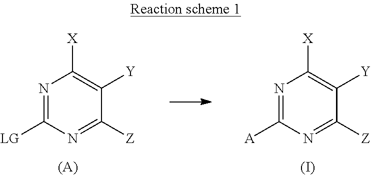

For example, a compound of formula (I) may be prepared by reacting a suitable metal or metalloid derivative A-M (for example, a boronic acid or ester, a trialkyltin derivative, a zinc derivative or a Grignard reagent) with a compound of formula (A) in the presence of a suitable base (for example an inorganic base, such as potassium phosphate or caesium fluoride, or an organic base, such as triethylamine), a metal source (for example, a palladium source such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water)—see reaction scheme 2. The metal catalyst and ligands may also be added as a single, pre-formed, complex (for example a palladium/phosphine complex, such as palladium tetrakistriphenylphosphine, bis(triphenylphosphine)palladium dichloride or [1,1′-bis(diphenylphosphino)ferrocene] palladium dichloride).

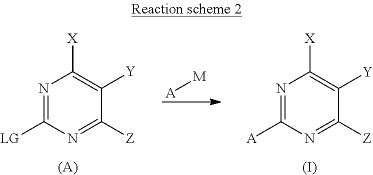

As an additional example (see reaction scheme 3) a compound of formula (I) in which A is an alkenyl group may be prepared using a Heck reaction in which the group A component containing the double bond may be reacted with a compound of formula (A) in the presence of a suitable metal catalyst (for example a palladium derivative, such as palladium acetate), optionally with a suitable ligand for the metal, and a suitable base (for example an inorganic base, such as potassium phosphate) in a suitable solvent (for example N-methylpyrrolidone).

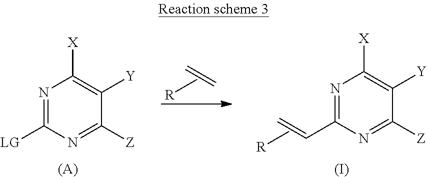

Alternatively, compounds of formula (I) may be prepared from compounds of formula (B), where M represents a suitable metal or metalloid derivative (for example a boronic acid or ester, a trialkyltin group, a suitably substituted silyl group, a zinc derivative or a magnesium halide) by reaction with a compound A-LG in which LG represents a leaving group such as a halogen atom or sulphonate (reaction scheme 4, below).

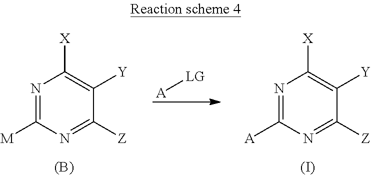

For example, a compound of formula (I) may be prepared from a compound of formula B in which M is a boronic acid group by reaction with a compound A-LG in the presence of a metal catalyst (for example a palladium derivative such as Pd₂(dba)₃), optionally with a suitable ligand (for example a phosphine such as X-Phos) and a base (for example potassium phosphate or caesium fluoride) in a suitable solvent.

Compounds of formula (B) may be prepared from other compounds of formula (B) using a transmetallation reaction. For example, a compound of formula (B) where M is a boronic acid may be prepared from a compound of formula (B) where M is a magnesium halide by reaction with a trialkylboronate, followed by hydrolysis (for example under acidic conditions).

Alternatively compounds of formula (B) may be prepared from compounds of formula (A), shown schematically below (reaction scheme 5).

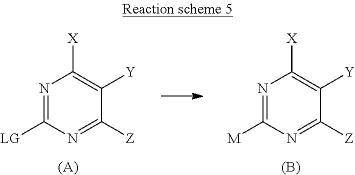

For example, a compound of formula (B) where M is a boronate ester or a trialkylstannane may be prepared from a compound of formula (A) by treating it with a suitable M-containing reagent (for example pinacolborane, bispinacolatodiboron, hexa-alkyldi-tin) in the presence of a metal catalyst (for example a palladium species, such as bis(diphenylphosphine)palladium dichloride) in a suitable solvent (for example dioxane).

Alternatively, a compound of formula (B) where M is a magnesium halide may be prepared from a compound of formula (A) by treatment with a suitable Grignard reagent (for example an isopropylmagnesium halide such as isopropylmagnesium chloride) in a suitable solvent.

Compounds of formula (A) may be prepared from compounds of formula (C) (reaction scheme 6), where LG′ is a second leaving group, which may be the same as or different to LG.

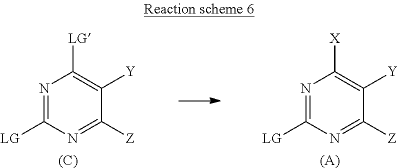

For example, a compound of formula (A) may be prepared from a compound of formula (C) by reaction with a reagent X—H or X⁻ in a suitable solvent (for example an ether solvent, such as tetrahydrofuran, or a polar aprotic solvent, such as dimethylsulphoxide). The reagent X⁻ may be generated in situ by addition of a suitable base to X—H, or it may be prepared separately and added as a suitable salt.

As an additional example a compound of formula (A) may be prepared from a compound of formula (C) by treatment with a reagent X—H in the presence of a suitable catalyst (for example a metal catalyst, such as a palladium source) and optionally a suitable ligand (for example a phosphine ligand, such as Josiphos) in a suitable solvent.

Compounds of formula (C1), which are compounds of formula (C) in which LG and LG′ are the same and are, for example, a halogen atom or a sulphonate) may be prepared from compounds of formula (D) (see reaction scheme 7 below).

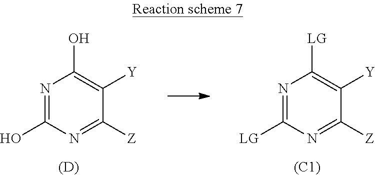

For example, a compound of formula (C1) in which LG is a halogen atom may be prepared from a compound of formula (D) by treatment with a suitable reagent (for example a phosphoryl halide such as phosphorous oxychloride) and optionally a suitable base (for example an organic base, such as N,N-diethylaniline).

Compounds of formula (C2) (that is compounds of formula (C) in which LG and LG′ are different) may be prepared from compounds of formula (E) in which B represents a suitable precursor group to LG′ (reaction scheme 8).

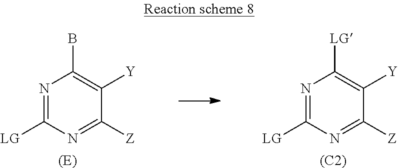

For example, a compound of formula (C2) in which LG′ is a sulphone may be prepared from a compound of formula (E1), which is a compound of formula (E) in which B is a thioether group, by reaction with a suitable oxidising agent, for example a peracid such as meta-chloroperbenzoic acid) (reaction scheme 9).

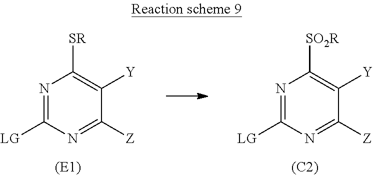

Compounds of formula (E) may be prepared from compounds of formula (C1) by treatment with a reagent B—H or B.

For example, a compound of formula (E1) may be prepared from a compound of formula (C1) by reaction with a thiol or thiolate anion, optionally in the presence of a suitable base, in a suitable solvent (see reaction scheme 10).

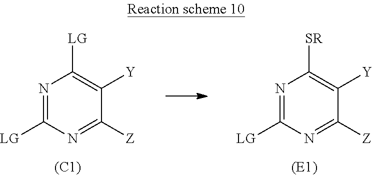

Compounds of formula (D) may be prepared from compounds of formula (F) by reaction with a suitable source of electropositive Y (reaction scheme 11).

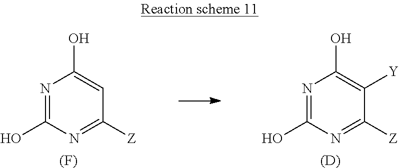

For example a compound of formula (D) in which Y is an acyl group may be prepared from a compound of formula (F) by reaction with an acyl halide (for example acetyl chloride) in the presence of a Lewis acid (for example aluminium trichloride) in a suitable solvent (for example nitrobenzene).

Alternatively, compounds of formula (D) may be prepared from compounds of formula (G) where D represents a suitable reactive group (see reaction scheme 12 below). Examples of such a reactive group are halogen atoms and sulphonates.

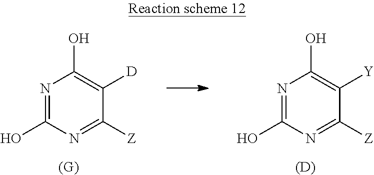

For example, such a transformation may be carried out by reaction with a metal or metalloid derivative of the group Y (for example a boronic acid or boronate ester) in the presence of a base (for example an inorganic base, such as potassium phosphate or caesium fluoride, or an organic base, such as triethylamine), a metal source (for example, a palladium source such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water). The metal catalyst and ligands may also be added as a single, pre-formed, complex (for example a palladium/phosphine complex, such as palladium tetrakistriphenylphosphine, bis(triphenylphosphine)palladium dichloride or [1,1′-bis(diphenylphosphino)ferrocene] palladium dichloride).

Compounds of formula (G) may be prepared from compounds of formula (F) by reaction with a suitable source of electropositive D (reaction scheme 13).

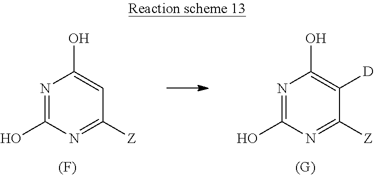

For example a compound of formula (G) in which D is a halogen may be prepared from a compound of formula (F) by reaction with an N-halosuccinimide (for example N-chlorosuccinimide) in a suitable solvent (for example dimethylformamide).

Compounds of formula (F) are well known in the literature, or can be made readily from compounds known in the literature by standard methods known to the skilled man.

Compounds of formula (I) may also be prepared from compounds of formula (H) where D represents a suitable reactive group (see reaction scheme 14 below). Examples of such a reactive group are halogen atoms and sulphonates.

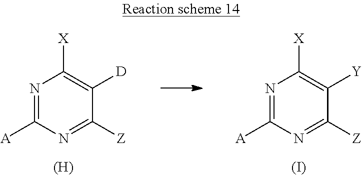

For example, such a transformation may be carried out by reaction with a metal or metalloid derivative of the group Y (for example a boronic acid or boronate ester) in the presence of a base (for example an inorganic base, such as potassium phosphate or caesium fluoride, or an organic base, such as triethylamine), a metal source (for example, a palladium source such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water). The metal catalyst and ligands may also be added as a single, pre-formed, complex (for example a palladium/phosphine complex, such as palladium tetrakistriphenylphosphine, bis(triphenylphosphine)palladium dichloride or [1,1′-bis(diphenylphosphino)ferrocene] palladium dichloride).

Compounds of formula (H) may be prepared from compounds of formula (I) (reaction scheme 15), where LG represents a suitable leaving group. Examples of such leaving groups are halogen atoms, sulphonates and sulphones.

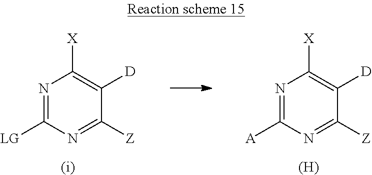

For example, a compound of formula (H1), which is a compound of formula (H) in which A is an alkylthio group, may be prepared by reacting a compound of formula (I) with an alkanethiolate (for example sodium methanethiolate) in a suitable solvent (for example a polar solvent, such as methanol). See reaction scheme 16 below.

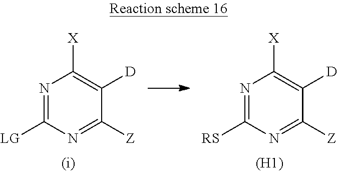

In a further example, a compound of formula (H) may be prepared by reacting a suitable metal or metalloid derivative A-M (for example, a boronic acid or ester, a trialkyltin derivative, a zinc derivative or a Grignard reagent) with a compound of formula (i) in the presence of a suitable base (for example an inorganic base, such as potassium phosphate or caesium fluoride, or an organic base, such as triethylamine), a metal source (for example, a palladium source such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water). See reaction scheme 17 below. The metal catalyst and ligands may also be added as a single, pre-formed, complex (for example a palladium/phosphine complex, such as palladium tetrakistriphenylphosphine, bis(triphenylphosphine)palladium dichloride or [1,1′-bis(diphenylphosphino)ferrocene] palladium dichloride).

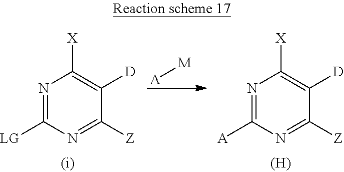

As an additional example, a compound of formula (H) in which A is an alkenyl group may be prepared using a Heck reaction in which the group A component containing the double bond may be reacted with a compound of formula (i) in the presence of a suitable metal catalyst (for example a palladium derivative, such as palladium acetate), optionally with a suitable ligand for the metal, and a suitable base (for example an inorganic base, such as potassium phosphate) in a suitable solvent (for example N-methylpyrrolidone); see reaction scheme 18.

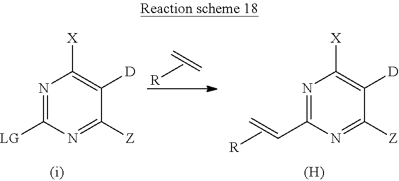

Compounds of formula (i) may be prepared from compounds of formula (J), where LG′ is a second leaving group which may be the same as or different to LG (reaction scheme 19).

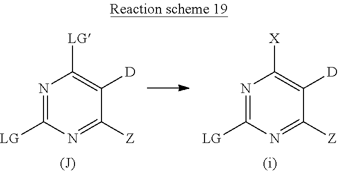

For example, a compound of formula (i) may be prepared from a compound of formula (J) by reaction with a reagent X—H or X⁻ in a suitable solvent (for example an ether solvent, such as tetrahydrofuran). The reagent X⁻ may be generated in situ by addition of a suitable base to X—H, or it may be prepared separately and added as a suitable salt.

As a further example, a compound of formula (i) may be prepared from a compound of formula (J) by treatment with a reagent X—H in the presence of a suitable catalyst (for example a metal catalyst, such as a palladium source) and optionally a suitable ligand (for example a phosphine ligand, such as Josiphos) in a suitable solvent.

Compounds of formula (J1), which are compounds of formula (J) in which LG and LG′ are the same and are, for example, a halogen atom or a sulphonate, may be prepared from compounds of formula (G) (see reaction scheme 20 below).

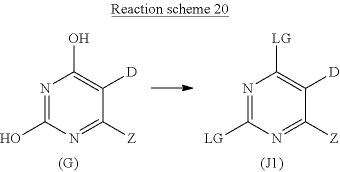

For example, a compound of formula (J1) in which LG is a halogen atom may be prepared from a compound of formula (G) by treatment with a suitable reagent (for example a phosphoryl halide such as phosphorous oxychloride) and optionally a suitable base (for example an organic base, such as N,N-diethylaniline).

Compounds of formula (I) may be prepared from compounds of formula (K) by reaction with a suitable source of electropositive Y (reaction scheme 21).

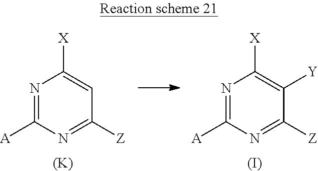

For example a compound of formula (I) in which Y is an acyl group may be prepared from a compound of formula (K) by reaction with an acyl halide (for example acetyl chloride) in the presence of a Lewis acid (for example aluminium trichloride) in a suitable solvent (for example nitrobenzene).

Compounds of formula (H) may also be prepared by reaction of compounds of formula (K) with a suitable source of electrophilic D (reaction scheme 22).

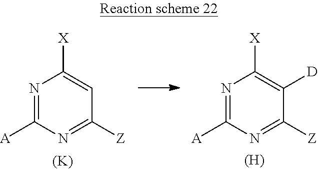

For example a compound of formula (H) in which D is a halogen may be prepared from a compound of formula (K) by reaction with an N-halosuccinimide (for example N-chlorosuccinimide) in a suitable solvent (for example dimethylformamide).

Compounds of formula (K) may be prepared from compounds of formula (L) (shown in reaction scheme 23 below), in which LG represents a leaving group.

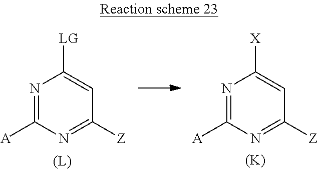

For example, a compound of formula (K) may be prepared from a compound of formula (L) by reaction with a reagent X—H or X⁻ in a suitable solvent (for example an ether solvent, such as tetrahydrofuran). The reagent X⁻ may be generated in situ by addition of a suitable base to X—H, or it may be prepared separately and added as a suitable salt.

As an additional example a compound of formula (K) may be prepared from a compound of formula (L) by treatment with a reagent X—H in the presence of a suitable catalyst (for example a metal catalyst, such as a palladium source) and optionally a suitable ligand (for example a phosphine ligand, such as Josiphos) in a suitable solvent.

Compounds of formula (L) may be prepared from compounds of formula (M) (reaction scheme 24 below).

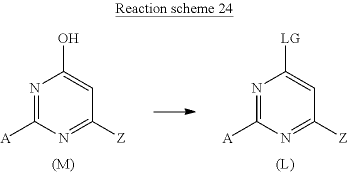

For example, a compound of formula (L) in which LG is a halogen atom may be prepared from a compound of formula M by treatment with a suitable reagent (for example a phosphoryl halide such as phosphorous oxychloride) and optionally a suitable base (for example an organic base, such as N,N-diethylaniline).

Compounds of formula (M) may be prepared from amidines of formula (N) by reaction with a suitable ketoester of formula (O) (reaction scheme 25)

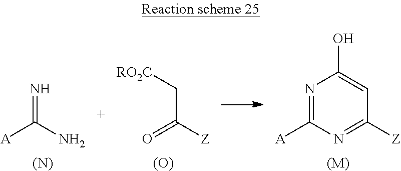

For example, a compound of formula (M1), which is a compound of formula (M) in which Z is a carboxylic acid or ester, may be prepared by the reaction of an amidine of formula (N) with an oxaloacetate diester of formula (O1), wherein (O1) is a compound of formula (O) in which Z is CO₂R, optionally in the presence of a suitable base (for example an inorganic base, such as sodium hydroxide), in a suitable solvent (for example water)—reaction scheme 26. The diester (O1) may also be used in the form of a salt, for example the sodium salt.

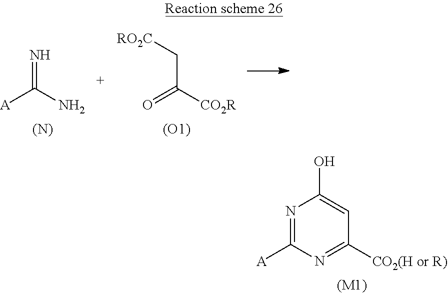

In an additional example, shown schematically below in reaction scheme 27, a compound of formula (M2) (i.e. a compound of formula (M) in which Z is an acetal group) may be prepared by the condensation of an amidine of formula (N) with a ketoester of formula (O2) (i.e. a compound of formula (O) in which Z is CH(OR)₂) in the presence of a base (e.g. an alkoxide base, such as sodium methoxide) in a suitable solvent (e.g. an alcohol, such as methanol).

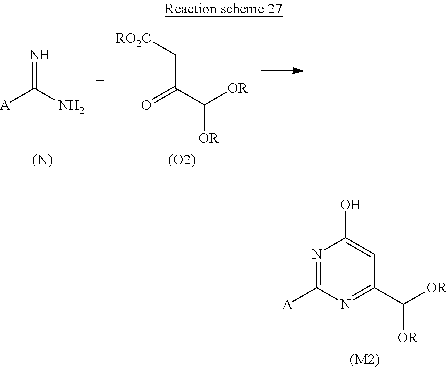

In general, amidines (N) and diesters (O) are either well known in the literature or may be prepared using standard methods with which the skilled man is familiar. Thus in a further aspect the invention provides the use of an amidine of formula N as defined above, as an intermediate in the preparation of a herbicide, in particular in the preparation of a compound of formula (I) as defined herein. However, we have found that novel amidines of formula (II) as defined hereinafter, are particularly useful as intermediates for use in the invention. The invention also provides an amidine of formula (II)

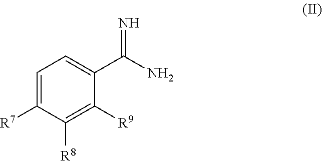

wherein R⁷ is methyl, Br, or Cl; R⁸ is H, F, Cl, OR¹⁰, or N(R¹⁰)₂; R⁹ is H, F, or Cl; each R¹⁰ is independently H or C₁₋₄alkyl, provided that i) R⁸ and R⁹ are not both hydrogen, (ii) when R⁷ is methyl and R⁹ is hydrogen, then R⁸ is not F, Cl, or NH₂, (iii) when R⁷ is Cl and R⁸ is hydrogen, R⁹ is not Cl, (iv) when R⁷ is Cl and R⁸ is Cl, R⁹ is not hydrogen, and (v) when R⁷ is Br, and R9 is hydrogen, R⁸ is not F. Particularly preferred compounds of formula (II) are those wherein R⁷ is methyl, Br, or Cl; R⁸ is F, Cl, OR¹⁰, or N(R¹⁰)₂; R⁹ is F, or Cl; and each R10 is independently H or C₁₋₄ alkyl. The skilled man will also appreciate that in many cases amidines of formula (II) may be in salt form, in particular in the form of the HCl salt. Such salts may be obtained routinely by standard methods.

Compounds of formula (K1) (i.e. compounds of formula K in which X is NH₂) may be prepared by the reaction of amidines of formula (N) with cyanoketones of formula (P) in the presence of a base (e.g. an alkoxide base, such as sodium methoxide) in a suitable solvent (e.g. an alcohol, such as ethanol)—see reaction scheme 28 below.

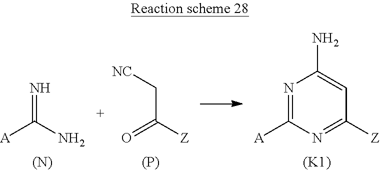

For example a compound of formula (K2), which is a compound of formula (K1) in which Z is CO₂R, may be prepared by the reaction of an amidine of formula (N) with a cyanopyruvate ester of formula (P1) (i.e. a compound of formula (P) in which Z is CO₂R). This is shown schematically in reaction scheme 29 below. In one example the compound of formula (P1) may be reacted first with an alkylating agent (e.g. a methylating agent, such as dimethyl sulphate) in the presence of a base (e.g. an inorganic base, such as sodium bicarbonate) to form an enol ether, which is then reacted with amidine (N) in the presence of a base (e.g. an alkoxide base, such as sodium methoxide).

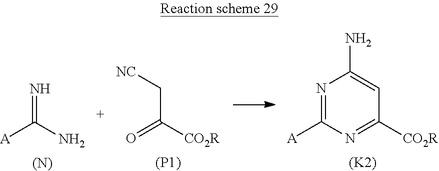

Compounds of formula (P) are known or may be prepared using routine, known methods.

Compounds of formula (H) may also be prepared from compounds of formula (Q) (shown in reaction scheme 30 below), in which LG represents a leaving group.

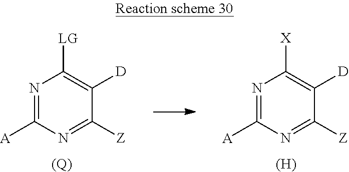

For example, a compound of formula (H) may be prepared from a compound of formula (Q) by reaction with a reagent X—H or X⁻ in a suitable solvent (for example an ether solvent, such as tetrahydrofuran, or a polar aprotic solvent, such as dimethylsulphoxide). The reagent X⁻ may be generated in situ by addition of a suitable base to X—H, or it may be prepared separately and added as a suitable salt.

As an additional example a compound of formula (H) may be prepared from a compound of formula (Q) by treatment with a reagent X—H in the presence of a suitable catalyst (for example a metal catalyst, such as a palladium source) and optionally a suitable ligand (for example a phosphine ligand, such as Josiphos) in a suitable solvent.

Compounds of formula (Q) may be prepared from compounds of formula (R) (reaction scheme 31 below).

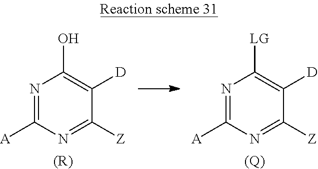

For example, a compound of formula (Q) in which LG is a halogen atom may be prepared from a compound of formula (R) by treatment with a suitable reagent (for example a phosphoryl halide such as phosphorous oxychloride) and optionally a suitable base (for example an organic base, such as N,N-diethylaniline).

Compounds of formula (R) may be prepared from compounds of formula (M) by reaction with a suitable source of electropositive D (reaction scheme 32 below).

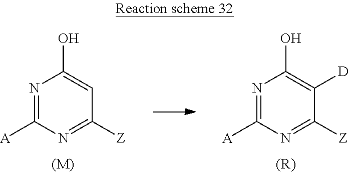

For example a compound of formula (R) in which D is a halogen may be prepared from a compound of formula (M) by reaction with an N-halosuccinimide (e.g. N-chlorosuccinimide) in a suitable solvent (e.g. dimethylformamide), or with a metal hypohalite (e.g. sodium hypochlorite) in a suitable solvent (e.g. acidic water).

Compounds of formula (I) may also be prepared from compounds of formula (S) (see reaction scheme 33), in which LG represents a leaving group (e.g. a halogen or sulphonate).

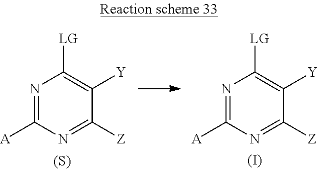

For example, a compound of formula (I) may be prepared from a compound of formula (S) by reaction with a reagent X—H or X⁻ in a suitable solvent (e.g. methanol, dimethylsulphoxide or water). The reagent X⁻ may be generated in situ by addition of a suitable base to X—H, or it may be prepared separately and added as a suitable salt.

As a further example, a compound of formula (I) may be prepared from a compound of formula (S) by treatment with a reagent X—H in the presence of a suitable catalyst (e.g. a metal catalyst, such as a palladium source) and optionally a suitable ligand (e.g. a phosphine ligand, such as Josiphos) in a suitable solvent.

Compounds of formula (S) may be prepared from compounds of formula (T) (reaction scheme 34).

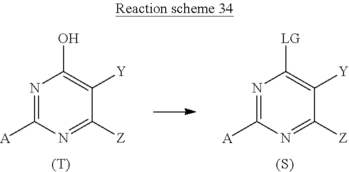

For example, a compound of formula (S) in which LG is a halogen atom may be prepared from a compound of formula (T) by treatment with a suitable reagent (e.g. a phosphoryl halide, such as phosphorous oxychloride) and optionally a suitable base (e.g. an organic base, such as N,N-diethylaniline).

Compounds of formula (T) may be prepared from amidines of formula (N) by reaction with a suitable ketoester of formula (U) (reaction scheme 35).

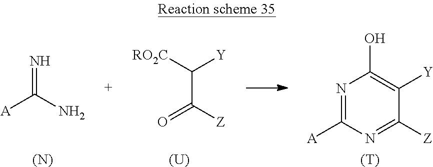

For example, a compound of formula (T1), which is a compound of formula (T) in which Z is a carboxylic acid or ester, may be prepared by the reaction of an amidine of formula (N) with an oxaloacetate diester of formula (U1) (i.e. a compound of formula (U) in which Z is CO₂R), optionally in the presence of a suitable base (e.g. an inorganic base, such as sodium hydroxide), in a suitable solvent (e.g. water). This is shown schematically below (reaction scheme 36). The diester (U1) may also be used in the form of a salt, for example the sodium salt.

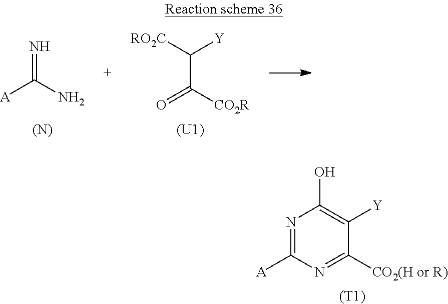

Compounds of formula (U) are known in the literature, or may be prepared from compounds of formula (O) by methods well known in the art (reaction scheme 37).

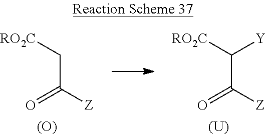

Compounds of formula (T) may be prepared by the condensation of amidines of formula (N) with substituted acetate esters of formula (V) and esters of formula (W) in the presence of a base (e.g. an alkoxide base, such as sodium ethoxide), in a suitable solvent (e.g. an alcohol, such as ethanol) (reaction scheme 38).

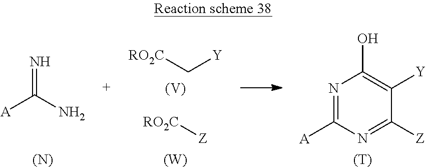

For example a compound of formula (T1) (as defined above) may be prepared by the reaction of an amidine of formula (N) with an acetate ester of formula (V) and a diethyl oxalate of formula (W1) (i.e. a compound of formula (W) in which Z is a carboxylate ester).

Compounds of formula (V) and (W) are readily available and well known in the literature.

Alternatively, compounds of formula (T) may be prepared from compounds of formula (M) by reaction with a suitable source of electropositive Y (reaction scheme 39).

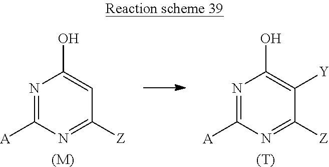

For example a compound of formula (T) in which Y is an acyl group may be prepared from a compound of formula (M) by reaction with an acyl halide (e.g. acetyl chloride) in the presence of a Lewis acid (e.g. aluminium trichloride) in a suitable solvent (e.g. nitrobenzene).

Compounds of formula (T) may also be prepared from compounds of formula (R) where D represents a suitable reactive group (reaction scheme 40). Examples of such a reactive group are halogen atoms and sulphonates.

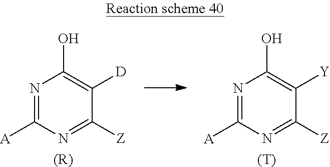

For example, such a transformation may be carried out by reaction with a metal or metalloid derivative of the group Y (e.g. a boronic acid or boronate ester) in the presence of a base (e.g. an inorganic base, such as potassium phosphate or caesium fluoride, or an organic base, such as triethylamine), a metal source (e.g. a palladium source such as Pd₂(dba)₃) and, optionally, a ligand for the metal (e.g. a phosphine ligand, such as X-Phos) in a suitable solvent (e.g. a single solvent, such as acetonitrile, or a mixed solvent system, such as a mixture of dimethoxyethane and water). The metal catalyst and ligands may also be added as a single, pre-formed, complex (e.g. a palladium/phosphine complex, such as palladium tetrakistriphenylphosphine, bis(triphenylphosphine)palladium dichloride or [1,1′-bis(diphenylphosphino)ferrocene] palladium dichloride).

Alternatively, compounds of formula (T) may be prepared from compounds of formula (X) (reaction scheme 41 below).

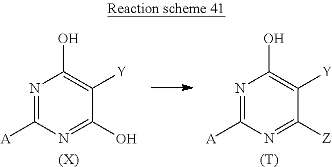

For example, a compound of formula (T2), which is a compound of formula (T) in which Z is O—(CHR^{w})ₙ—COR^{cb} (wherein R^{w}, n and R^{cb} are as defined hereinbefore), may be prepared from a compound of formula (X) by reaction with a compound of formula (Y) in the presence of a base e.g. sodium hydride, in a suitable solvent e.g. an ether, such as tetrahydrofuran (reaction scheme 42).

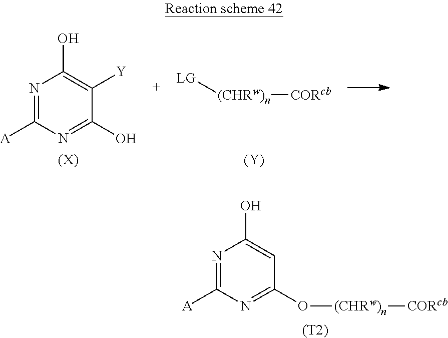

Compounds of formula (X) may be prepared by the reaction of amidines of formula (N) with malonyl diesters of formula (Z) in the presence of a suitable base (e.g. an inorganic base, such as potassium carbonate, or an alkoxide base, such as sodium methoxide) in a suitable solvent (e.g. an ether, such as diglyme, or an alcohol, such as ethanol) (reaction scheme 43).

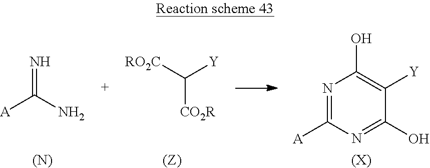

Diesters of formula (Z) are known in the literature or may be prepared by methods well known in the literature.

Compounds of formula (X) may be prepared from compounds of formula (AA) by reaction with a suitable source of electropositive Y (reaction scheme 44).

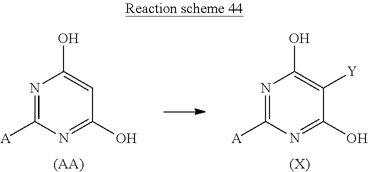

For example a compound of formula (X) in which Y is an acyl group may be prepared from a compound of formula (AA) by reaction with an acyl halide e.g. acetyl chloride, in the presence of a Lewis acid e.g. aluminium trichloride, in a suitable solvent e.g. nitrobenzene.

Alternatively, compounds of formula (X) may be prepared from compounds of formula (AB) where D represents a suitable reactive group (reaction scheme 45). Examples of such a reactive group are halogen atoms and sulphonates.

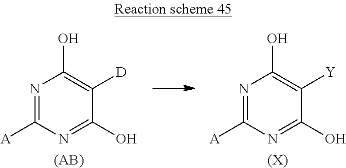

For example, such a transformation may be carried out by reaction with a metal or metalloid derivative of the group Y (e.g. a boronic acid or boronate ester), in the presence of a base (e.g. an inorganic base, such as potassium phosphate or caesium fluoride, or an organic base, such as triethylamine) a metal source (e.g. a palladium source such as Pd₂(dba)₃), and, optionally, a ligand for the metal (e.g. a phosphine ligand, such as X-Phos), in a suitable solvent (e.g. a single solvent, such as acetonitrile, or a mixed solvent system, such as a mixture of dimethoxyethane and water). The metal catalyst and ligands may also be added as a single, pre-formed, complex (e.g a palladium/phosphine complex, such as palladium tetrakistriphenylphosphine, bis(triphenylphosphine)palladium dichloride or [1,1′-bis(diphenylphosphino)ferrocene] palladium dichloride).

Compounds of formula (AB) may be prepared by reaction of compounds of formula (AA) with a suitable source of electropositive D (reaction scheme 46).

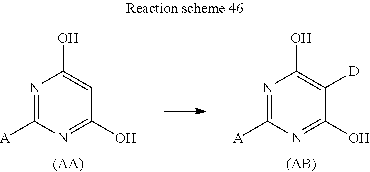

For example a compound of formula (AB) in which D is a halogen may be prepared from a compound of formula (AA) by reaction with a halogenating agent (e.g. an N-halosuccinimide such as N-chlorosuccinimide, or an elemental halogen such as bromine).

Compounds of formula (AA) may be prepared from amidines of formula (N) and malonyl diesters of formula (AC) (reaction scheme 47).

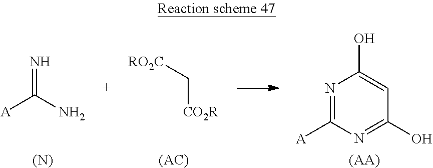

Diesters of formula (AC) are well known in the literature.

Compounds of formula (S) may be prepared from compounds of formula (AD) in which B represents a suitable precursor group to LG (reaction scheme 48).

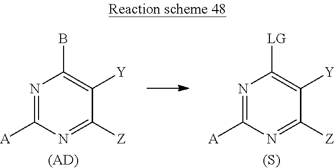

For example, a compound of formula (S) in which LG is a sulphone may be prepared from a compound of formula (AD1), which is a compound of formula (AD) in which B is a thioether group, by reaction with a suitable oxidising agent, for example a peracid such as meta-chloroperbenzoic acid) (reaction scheme 49).

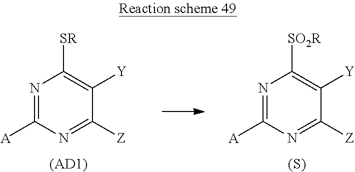

Compounds of formula (AD) may be prepared from compounds of formula (AE) where D represents a suitable reactive group (reaction scheme 50). Examples of such a reactive group are halogen atoms and sulphonates.

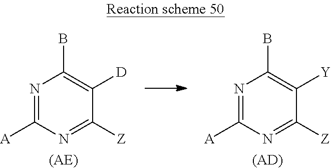

For example, such a transformation may be carried out by reaction with a metal or metalloid derivative of the group Y (e.g. a boronic acid or boronate ester) in the presence of a base (e.g. an inorganic base, such as potassium phosphate or caesium fluoride, or an organic base, such as triethylamine), a metal source (e.g. a palladium source such as Pd₂(dba)₃) and, optionally, a ligand for the metal (e.g. a phosphine ligand, such as X-Phos) in a suitable solvent (e.g. a single solvent, such as acetonitrile, or a mixed solvent system, such as a mixture of dimethoxyethane and water). The metal catalyst and ligands may also be added as a single, pre-formed, complex (e.g. a palladium/phosphine complex, such as palladium tetrakistriphenylphosphine, bis(triphenylphosphine)palladium dichloride or [1,1′-bis(diphenylphosphino)ferrocene] palladium dichloride).

Compounds of formula (AE) may be prepared from compounds of formula (Q) by treatment with a reagent B—H or B⁻.

For example, a compound of formula (AE1), which is a compound of formula (AE) in which B is a thioether, may be prepared from a compound of formula (Q) by reaction with a thiol or thiolate anion, optionally in the presence of a suitable base, in a suitable solvent (see reaction scheme 51).

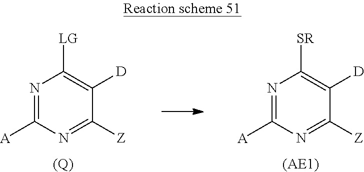

Compounds of formula (S) may be prepared from compounds of formula (AF), where LG and LG′ (which may be the same or different) are suitable leaving groups, such as halogen atoms or sulphonates (reaction scheme 52).

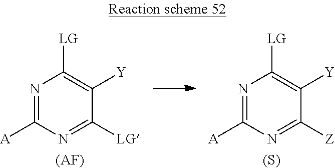

For example a compound of formula (S) in which Z is CO₂R may be prepared from a compound of formula (AF) by reaction with an alcohol ROH and carbon monoxide in the presence of a suitable metal catalyst (e.g. a palladium reagent, such as bis(triphenylphosphine)palladium dichloride) and a suitable base (e.g. an organic base, such as triethylamine). See reaction scheme 53 below. The reaction may conveniently be conducted in an atmosphere of carbon monoxide gas at atmospheric or raised pressure.

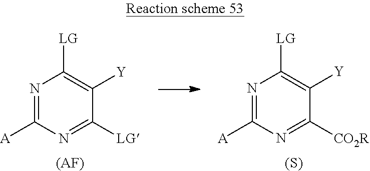

Alternatively (see reaction scheme 54 below) a compound of formula (S) in which Z is O—(CH^{w})ₙ—COR^{cb} may be prepared from a compound of formula (AF) by reaction with a compound of formula (AG) in the presence of a base (e.g. sodium hydride) in a suitable solvent (e.g. an ether, such as tetrahydrofuran).

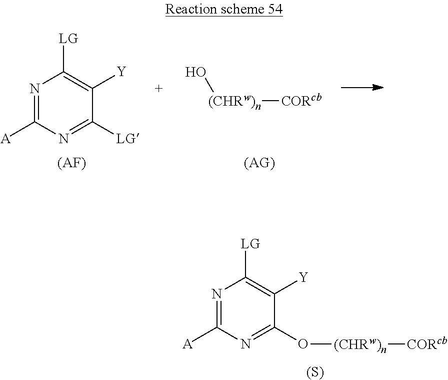

Compounds of formula (AG) are known compounds or may be prepared from known compounds using methods that are well known in the literature.

Compounds of formula (AF1), which are compounds of formula (AF) in which LG is the same as LG′, may be prepared from compounds of formula (X) by reaction with a suitable reagent, for example a phosphoryl halide or sulphonyl anhydride (reaction scheme 55).

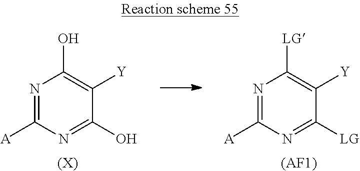

For example, a compound of formula (AF1) in which LG and LG′ are halogen atoms may be prepared by reaction of a compound of formula (X) with a halogenating agent (e.g. a phosphoryl halide such as phosphorus oxychloride) in the presence of a suitable base (e.g. an organic base, such as N,N-diethylaniline).

Compounds of formula (I) may be prepared from compounds of formula (AH), where LG is a suitable leaving group, such as a halogen atom or sulphonate (reaction scheme 56).

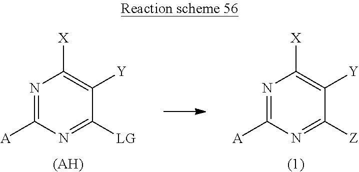

For example a compound of formula (I) in which Z is CO₂R may be prepared from a compound of formula (AH) by reaction with an alcohol ROH and carbon monoxide in the presence of a suitable metal catalyst e.g. a palladium reagent, such as bis(triphenylphosphine)palladium dichloride, and a suitable base e.g. an organic base, such as triethylamine (see reaction scheme 57). The reaction may conveniently be conducted in an atmosphere of carbon monoxide gas at atmospheric or raised pressure.

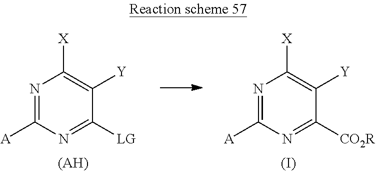

Alternatively a compound of formula (I) in which Z is O—(CHR^{w})ₙ—COR^{cb} (as defined hereinbefore) may be prepared from a compound of formula (AH) by reaction with a compound of formula (AG) in the presence of a base e.g. sodium hydride in a suitable solvent e.g. an ether, such as tetrahydrofuran (reaction scheme 58).

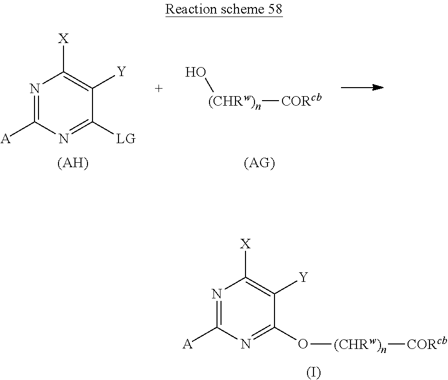

Compounds of formula (AH) may be prepared from compounds of formula (AF) (reaction scheme 59).

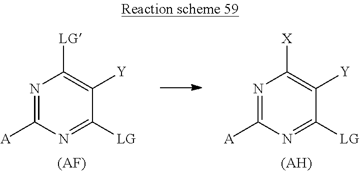

Compounds of formula (I) in which A is a ring may also be prepared from compounds of formula (AI) wherein E represents a suitable cyclisation precursor, by reactions in which ring A is formed (reaction scheme 60). Examples of suitable cyclisation precursors include groups containing carbon-carbon double or triple bonds, such as alkenes and alkynes.

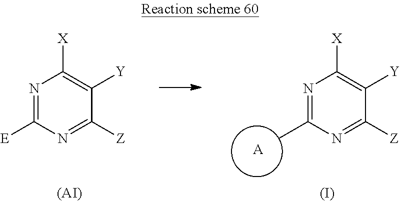

For example, a compound of formula (I) in which A is an unsaturated 6-membered ring may be prepared from a compound of formula (AI1) i.e. a compound of formula (AI) in which E is an alkyne, by reaction with a suitable diene (AJ) (reaction scheme 61 below).

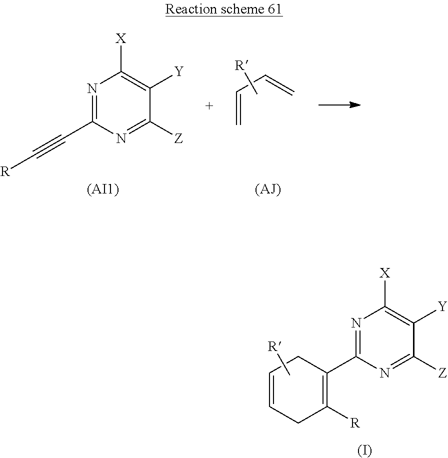

In a further example, a compound of formula (I) in which A is a cyclopropane may be prepared from a compound of formula (AI2) i.e. a compound of formula (I) in which A is an alkene, by reaction with a suitable cyclopropanation reagent (e.g. a reagent combination that generates a carbene, such as diiodomethane and diethyl zinc) (reaction scheme 62).

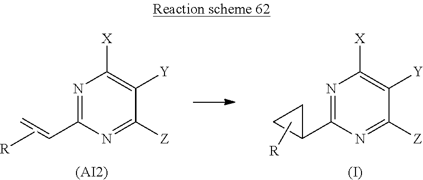

In an additional example a compound of formula (I) in which A is a 4-membered ring may be prepared from a compound of formula (AI2) by reaction with a suitable reagent (AK) containing a carbon-carbon double bond, for example an alkene.

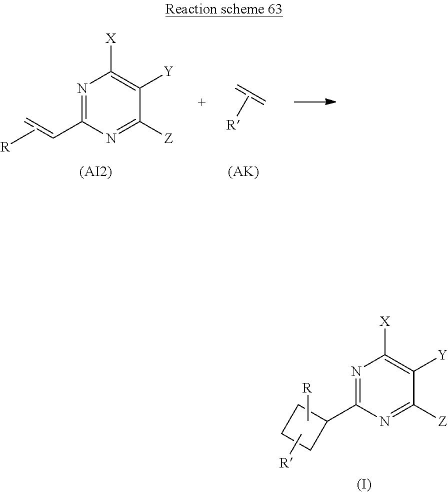

In another example, a compound of formula (I) in which A is a 5-membered ring may be formed from a compound of formula (AI2) by reaction with a suitable 1,3-dipolar species (AL) such as a trimethylenemethane species (reaction scheme 64).

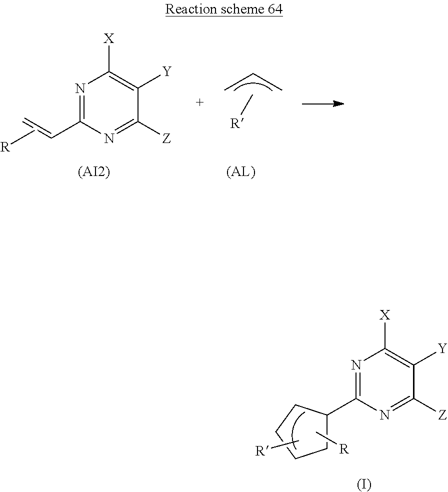

As a further example, a compound of formula (I) in which A is an unsaturated 6-membered ring may be prepared from a compound of formula (AI2) by reaction with a suitable diene (AJ) (reaction scheme 65).

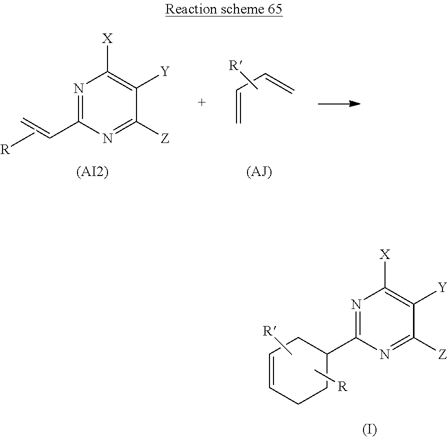

Compounds of formulae (AJ), (AK) and (AL) are known in the art, or can be made readily using methods that are well known in the literature.

Compounds of formula (AI1) may be prepared from compounds of formula (A) by a Sonogashira-type reaction with an alkyne (AM) (reaction scheme 66).

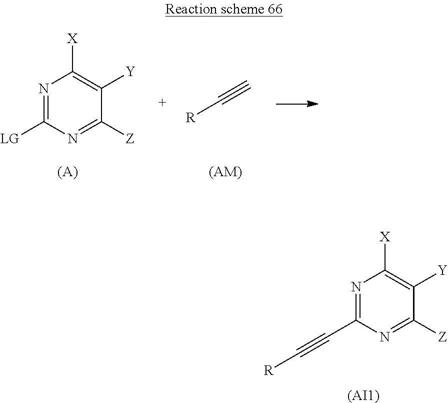

Compounds of formula (I) in which A is an alkene may be prepared from compounds of formula (AI3), which are compounds of formula (AI) in which E is an aldehyde or ketone group, by olefination reactions that are well known in the literature, for example the Wittig, Peterson, Tebbe and Petasis reactions (see reaction scheme 67, below).

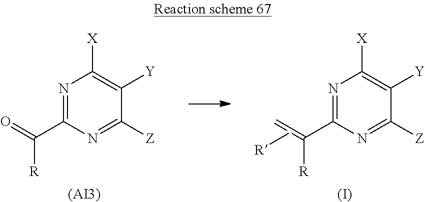

Compounds of formula (AI3) may be prepared from compounds of formula (AI2) by oxidative cleavage of the double bond (reaction scheme 68), for example by treatment with ozone.

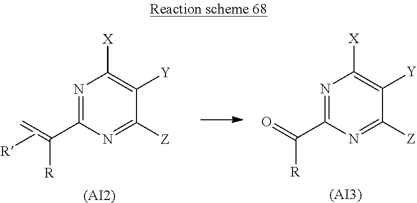

Alternatively compounds of formula (AI3) may be prepared from compounds of formula (AI4), which are compounds of formula (AI) in which E is a 1,2-diol group, by treatment with a suitable oxidising agent, for example sodium periodate (reaction scheme 69).

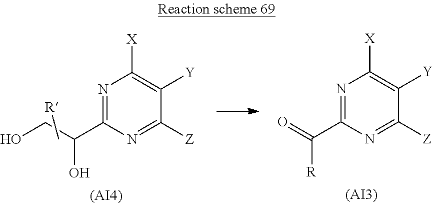

Compounds of formula (AI4) may be prepared from compounds of formula (AI2) by reaction with a suitable dihydroxylation reagent (e.g. an osmium species such as osmium tetroxide) (reaction scheme 70 below).

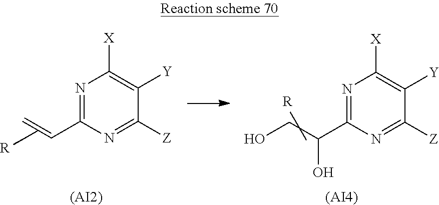

Compounds of formula (I) in which X is NH₂ may be prepared by the condensation of amidines of formula (N) with substituted acetonitriles of formula (AN) and esters of formula (W) (see reaction scheme 71 below). These reactions occur in the presence of a base (e.g. an alkoxide base, such as sodium ethoxide) in a suitable solvent (e.g. an alcohol, such as ethanol).

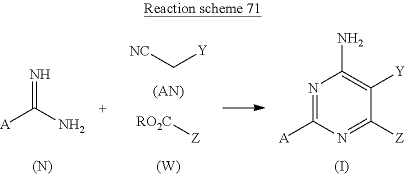

For example, a compound of formula (I) in which Z is CO₂R may be prepared by the reaction of an amidine of formula (N) with a substituted acetonitrile of formula (AN) and an oxalate diester of formula (W1). Compounds of formula (AN) are known in the literature.

Compounds of formula (I) in which m is 0 and n is 2 may be prepared by the reaction of compounds of formula (AO) with a reagent that can functionalise the double bond (reaction scheme 72).

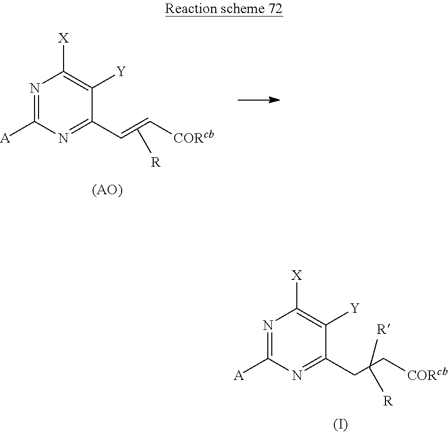

For example, a compound of formula (I) in which R′ is hydrogen may be prepared from a compound of formula (AO) by reaction with a suitable reducing agent (e.g. hydrogen gas in the presence of a metal catalyst, such as palladium supported on carbon) (reaction scheme 73).

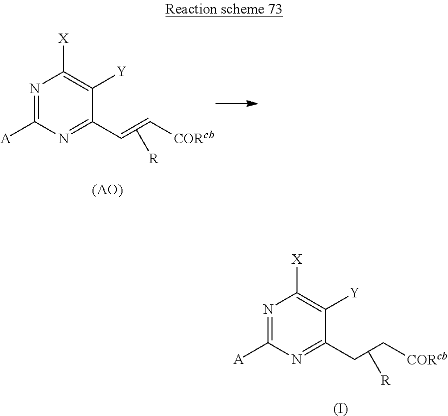

As a further example, a compound of formula (I) in which R′ represents vicinal hydroxyl groups may be prepared from a compound of formula (AO) by reaction with a dihydroxylation reagent (e.g. osmium tetroxide) (see reaction scheme 74 below).

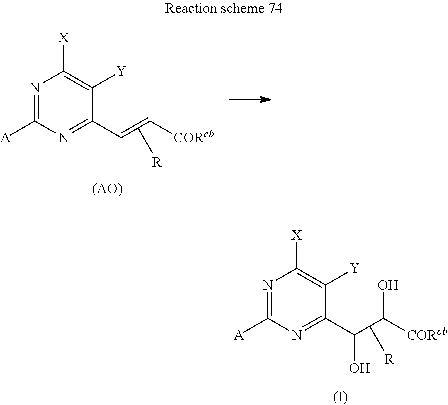

Compounds of formula (AO) may be prepared by the Heck reaction of compounds of formula (AH) with compounds of formula (AP) in the presence of a suitable metal catalyst (e.g. a palladium species, such as palladium acetate) and a base (e.g. an organic base, such as triethylamine), in a suitable solvent (reaction scheme 75).

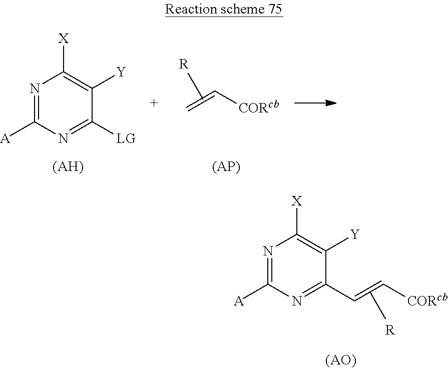

Compounds of formula (I) may be prepared from different compounds of formula (I) by the conversion of any of the substituents X, Y, Z or A into a different group X, Y, Z or A using techniques that are known in the literature and with which the skilled man will be familiar.

For example, a compound of formula (I) in which Y is an alkyl group may be prepared from a compound of formula (I) in which Y is an alkenyl or alkynyl group by reduction under suitable conditions (see reaction schemes 76, 77). Examples of such conditions include the use of hydrogen gas in the presence of a suitable catalyst, e.g. a metal catalyst, such as for example, palladium supported on carbon.

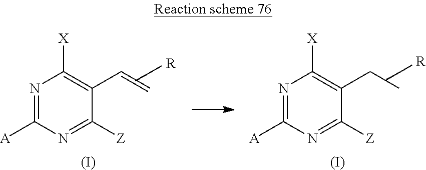

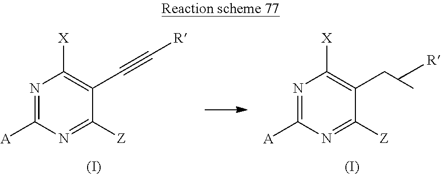

A compound of formula (I) in which Y is an alkenyl group may be prepared from a compound of formula (I) in which Y is an alkynyl group by reduction under suitable conditions (reaction scheme 78). Such conditions include reduction using hydrogen gas in the presence of an appropriate metal catalyst, for example a poisoned palladium metal catalyst such as Lindlars catalyst.

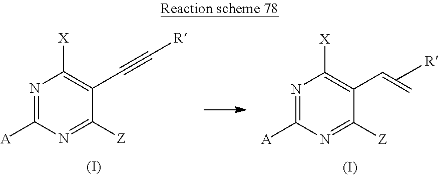

A compound of formula (I) in which Y is an acyl group may be prepared from a compound of formula (I) in which Y is an enol ether by hydrolysis (reaction scheme 79), for example using an aqueous acid.

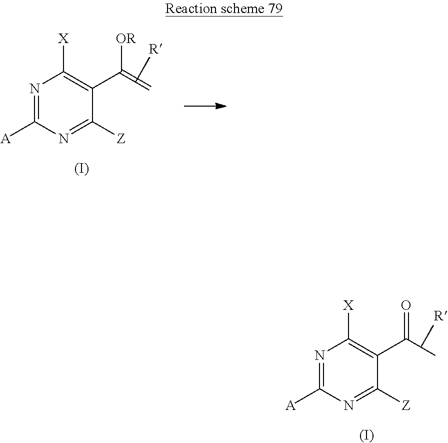

In an additional example, a compound of formula (I) in which Y is an alkene may be prepared from a compound of formula (I) in which Y is an aldehyde or ketone by using a suitable olefination reaction (reaction scheme 80), for example a Wittig, Horner-Emmons, Peterson or Tebbe reaction.

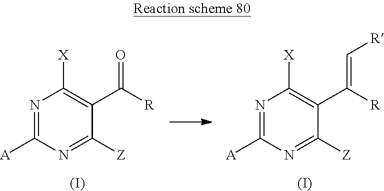

In a further example, a compound of formula (I) in which Y is a cyclopropane may be prepared from a compound of formula (I) in which Y is an alkene by reaction with a suitable cyclopropanation reagent e.g. a reagent combination that generates a carbene, such as diiodomethane and diethyl zinc (reaction scheme 81).

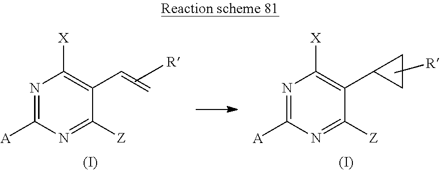

A compound of formula (I) in which A is an aromatic or heteroaromatic ring may be prepared from a compound of formula (I) in which A is an alkylthio group (see reaction scheme 82 below).

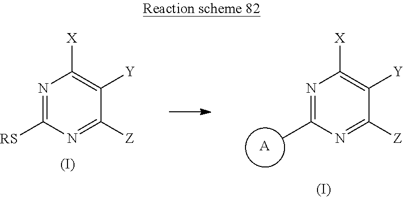

For example, such a transformation may be carried out by reaction with a metal or metalloid derivative of the ring A (for example a boronic acid or boronate ester) in the presence of a metal source (for example, a palladium source such as Pd₂(dba)₃) and, optionally, a ligand for the metal (for example a phosphine ligand, such as tri(2-furyl)phosphine), a further metal source (for example a copper complex, such as copper thiophene-2-carboxylate) in a suitable solvent (for example an ether, such as tetrahydrofuran). The metal catalyst and ligands may also be added as a single, pre-formed, complex (for example a palladium/phosphine complex, such as palladium tetrakistriphenylphosphine, bis(triphenylphosphine)palladium dichloride or [1,1′-bis(diphenylphosphino)ferrocene] palladium dichloride).

In a further example, an unsaturated group A (e.g. an alkene or cycloalkene) may be reduced to form a saturated group (e.g. an alkyl or cycloalkyl group). When A is an unsaturated ring it may be oxidised to form an aromatic ring under standard conditions.

A compound of formula (I) in which R⁵ is not hydrogen may be prepared from a compound of formula (I) in which R⁵ is H by reaction with a suitable reagent R⁵-LG in which LG is a leaving group such as a halogen atom. Examples of such reagents R⁵-LG are alkyl halides and acid anhydrides. For example a compound of formula (I) in which R⁵ is COR may be prepared from a compound of formula (I) in which R⁵ is H by reaction with an acylating agent such as an acyl chloride in the presence of a base (see reactjon scheme 83 below).

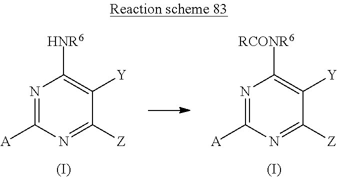

Alternatively a compound of formula (I) in which R⁵ is not hydrogen may be prepared from a compound of formula (I) in which R⁵ is H by reductive amination, involving first a condensation with a carbonyl compound such as an aldehyde or ketone and then reduction of the intermediate imine or iminium ion with a suitable reducing agent such as a metal hydride (e.g. sodium cyanoborohydride).

In an additional example a compound of formula (I) in which R^{cb} is OH may be prepared from a compound of formula (I) in which R^{cb} is OR, by hydrolysis under basic or acidic conditions, for example by treatment with aqueous sodium hydroxide (reaction scheme 84). Alternatively this transformation may be achieved by treatment of the ester with a nucleophile, for example an alkyl thiolate, in a suitable solvent.

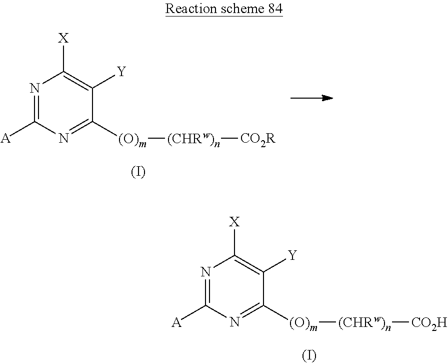

A compound of formula I in which R^{cb} is OR may be prepared directly from a compound of formula I in which R^{cb} is OH by esterification under standard conditions, for example by treatment with an alcohol ROH and an acid catalyst (for example, thionyl chloride). Alternatively, this transformation may be achieved by first preparing an activated derivative of the acid group, for example an acyl halide, followed by reaction with an alcohol.

Other derivatives of the acid group in compounds of formula (I) in which R^{cb} is OH may be prepared by standard methods found in the literature. For example a compound of formula (I) in which R^{cb} is NH₂ may be prepared from a compound of formula (I) in which R^{cb} is OH by treatment with a suitable coupling reagent (e.g. a carbodiimide, such as dicyclohexylcarbodiimide) and ammonia, optionally with an additive (e.g. dimethylaminopyridine), in a suitable solvent (e.g. dimethylformamide)—see reaction scheme 85. Alternatively, this transformation may be performed by first preparing an activated derivative of the carboxylic acid group (e.g. an acyl halide such as an acid chloride), and then treating the activated derivative with ammonia.

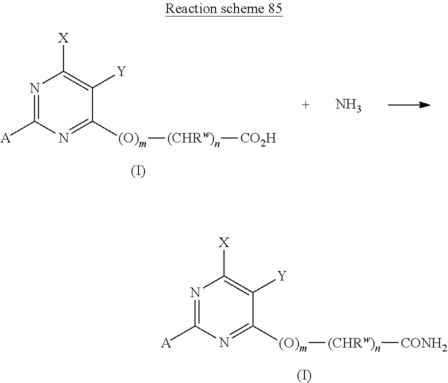

One skilled in the art will understand that transformations of this type may equally well be conducted at different stages of the synthetic route, for example converting one compound of formula (T) into a different compound of formula (T).

The person skilled in the art will also understand that in certain instances more than one transformation can be conducted at a time, utilising the same reaction conditions. For example a compound of formula (I) in which A and Y are the same may be prepared from a compound of formula (i) by reaction with an excess of a metal or metalloid derivative of A, such as a boronic acid, in the presence of a metal catalyst (e.g. a palladium derivative such as Pd₂(dba)₃), a ligand (e.g. a phosphine ligand such as X-Phos) and a base (such as, for example, potassium phosphate) in a suitable solvent (reaction scheme 86).

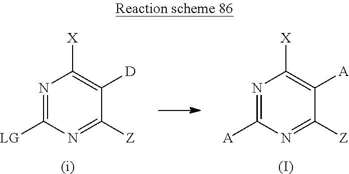

Another example is the preparation of a compound of formula (I) in which R^{cb} is NH₂ from a compound of formula (S1) (i.e. a compound of formula (S) in which R^{cb} is OR) by treatment with an excess of ammonia in a suitable solvent (see reaction scheme 87 below).

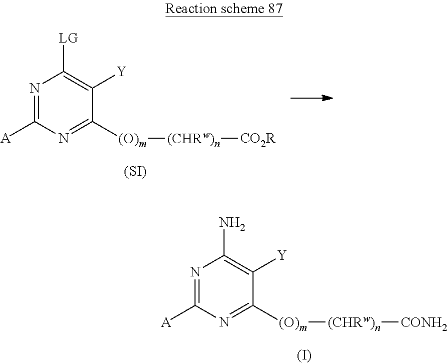

A further example is the preparation of a compound of formula (R1) (i.e. a compound of formula (R) in which Z is CO₂R), from a compound of formula (M2) by oxidation using a reagent that also provides a source of electropositive D (for example, N-bromosuccinimide for the case in which D=Br; see reaction scheme 88).

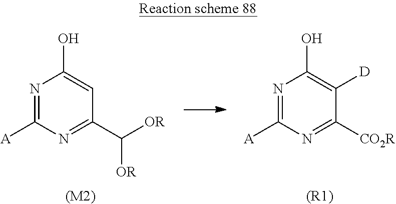

The skilled man will appreciate that it is often possible to alter the order in which the transformations above are conducted or to combine them in alternative ways to prepare a wide range of compounds of formula (I).

One skilled in the art will also realise that some reagents will be incompatible with certain values or combinations of substituents X, Y, Z and A. Accordingly additional steps, such as protection and deprotection steps, will be necessary to achieve the desired transformation, and the skilled man will immediately recognise where this is the case.

Compounds of formula (I) may be used in unmodified form, i.e. as obtainable from synthesis, but preferably are formulated in any suitable manner using formulation adjuvants, such as carriers, solvents and surface-active substances, for example, as described hereinafter. The invention thus extends to herbicidal compositions and/or formulations comprising a compound of the invention and at least one agriculturally acceptable formulation adjuvant or diluent.

The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO Specifications for Plant Protection Products, 5th Edition, 1999. The formulations can be in the form of concentrates which are diluted prior to use, although ready-to-use formulations can also be made. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof. The active ingredients can also be contained in very fine microcapsules consisting of a polymer. Microcapsules usually have a diameter of from 0.1 to 500 microns. Typically, they will contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other known polymers. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octa-decanoic acid, octylamine acetate, oleic acid, oleylamine, o-xylene, phenol, polyethylene glycol (PEG), propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like. Water is generally the carrier of choice for diluting the concentrates. Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances, as described, for example, in CFR 180.1001. (c) & (d).

A large number of surface-active substances may advantageously be used in the formulations, especially in those formulations designed to be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in “McCutcheon's Detergents and Emulsifiers Annual” MC Publishing Corp., Ridgewood N.J., 1981.

Further adjuvants that can usually be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and also liquid and solid fertilisers.

The compositions according to the invention can additionally include an additive comprising an oil of vegetable or animal origin, a mineral oil, alkyl esters of such oils or mixtures of such oils and oil derivatives. The amount of oil additive in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsified vegetable oil, such as AMIGO® (Rhone-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈₋₂₂ fatty acids, especially the methyl derivatives of C₁₂₋₁₈ fatty acids, for example the methyl esters of lauric acid, palmitic acid and oleic acid, being of importance. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000. Another preferred adjuvant is Adigor® (Syngenta AG) which is a methylated rapeseed oil-based adjuvant.

The application and action of the oil additives can be further improved by combination with surface-active substances, such as non-ionic, anionic or cationic surfactants. Examples of suitable anionic, non-ionic and cationic surfactants are listed on pages 7 and 8 of WO97/34485. Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂₋₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltriloxanes which are commercially available e.g. as Silwet L-77®, and also perfluorinated surfactants. The concentration of the surface-active substances in relation to the total additive is generally from 1 to 30% by weight. Examples of oil additives consisting of mixtures of oil or mineral oils or derivatives thereof with surfactants are Edenor ME SU®, Turbocharge® (Syngenta AG, CH) or ActipronC (BP Oil UK Limited, GB).

If desired, it is also possible for the mentioned surface-active substances to be used in the formulations on their own, that is to say without oil additives.

Furthermore, the addition of an organic solvent to the oil additive/surfactant mixture may contribute to an additional enhancement of action. Suitable solvents are, for example, Solvesso® (ESSO) or Aromatic Solvent® (Exxon Corporation). The concentration of such solvents can be from 10 to 80% by weight of the total weight. Oil additives that are present in admixture with solvents are described, for example, in U.S. Pat. No. 4,834,908. A commercially available oil additive disclosed therein is known by the name MERGE® (BASF Corporation). A further oil additive that is preferred according to the invention is SCORE® (Syngenta Crop Protection Canada).

In addition to the oil additives listed above, for the purpose of enhancing the action of the compositions according to the invention it is also possible for formulations of alkylpyrrolidones (e.g. Agrimax®) to be added to the spray mixture. Formulations of synthetic lattices, e.g. polyacrylamide, polyvinyl compounds or poly-1-p-menthene (e.g. Bond®, Courier® or Emerald®) may also be used. It is also possible for solutions that contain propionic acid, for example Eurogkem Pen-e-trate®, to be added to the spray mixture as action-enhancing agent.

Herbicidal compositions of the invention generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. Whereas commercial products will preferably be formulated as concentrates, the end user will normally employ dilute formulations.

Examples of preferred formulation types and their typical compositions are given below (% is percent by weight). Wettable powders as described herein are one particularly preferred type of formulation for use in the invention. In other preferred embodiments, in particular where the compound/composition/formulation of the invention is intended for use on turf, granular (inert or fertiliser) formulations as described herein are particularly suitable.

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%
The following Examples further illustrate, but do not limit, the invention.

### Formulation Examples for Herbicides of Formula (I) (%=% by Weight)

| F1. Emulsifiable concentrates | a) | b) | c) | d) |
|---|---|---|---|---|
| active ingredient | 5% | 10% | 25% | 50% |
| calcium dodecylbenzenesulfonate | 6% | 8% | 6% | 8% |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| NMP | — | — | 10% | 20% |
| arom. hydrocarbon mixture | 85% | 78% | 55% | 16% |
| (C₉-C₁₂) | | | | |

Emulsions of any desired concentration can be obtained from such concentrates by dilution with water.

| F2. Solutions | a) | b) | c) | d) |
|---|---|---|---|---|
| active ingredient | 5% | 10% | 50% | 90% |
| 1-methoxy-3-(3-methoxy- | — | 20% | 20% | — |
| propoxy)-propane | | | | |
| polyethylene glycol | 20% | 10% | — | — |
| MW 400 | | | | |
| NMP | — | — | 30% | 10% |
| arom. hydrocarbon mixture | 75% | 60% | — | — |
| (C₉-C₁₂) | | | | |

The solutions are suitable for use in the form of microdrops.

| F3. Wettable powders | a) | b) | c) | d) |
|---|---|---|---|---|
| active ingredient | 5% | 25% | 50% | 80% |
| sodium lignosulfonate | 4% | — | 3% | — |
| sodium lauryl sulphate | 2% | 3% | — | 4% |
| sodium | — | 6% | 5% | 6% |
| diisobutylnaphthalene- | | | | |
| sulfonate | | | | |
| octylphenol polyglycol | — | 1% | 2% | — |
| ether (7-8 mol of ethylene | | | | |
| oxide) | | | | |
| highly dispersed silicic acid | 1% | 3% | 5% | 10% |
| kaolin | 88% | 62% | 35% | — |

The active ingredient is mixed thoroughly with the adjuvants and the mixture is thoroughly ground in a suitable mill, affording wettable powders which can be diluted with water to give suspensions of any desired concentration.

| F4. Coated granules | a) | b) | c) |
|---|---|---|---|
| active ingredient | 0.1% | 5% | 15% |
| highly dispersed silicic acid | 0.9% | 2% | 2% |
| inorganic carrier | 99.0% | 93% | 83% |
| (diameter 0.1-1 mm) | | | |
| e.g. CaCO₃ or SiO₂ | | | |

The active ingredient is dissolved in methylene chloride and applied to the carrier by spraying, and the solvent is then evaporated off in vacuo.

| F5. Coated granules | a) | b) | c) |
|---|---|---|---|
| active ingredient | 0.1% | 5% | 15% |
| polyethylene glycol MW 200 | 1.0% | 2% | 3% |
| highly dispersed silicic acid | 0.9% | 1% | 2% |
| inorganic carrier | 98.0% | 92% | 80% |
| (diameter 0.1-1 mm) | | | |
| e.g. CaCO₃ or SiO₂ | | | |

The finely ground active ingredient is uniformly applied, in a mixer, to the carrier moistened with polyethylene glycol. Non-dusty coated granules are obtained in this manner.

| F6. Extruder granules | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants, and the mixture is moistened with water. The mixture is extruded and then dried in a stream of air.

| F7. Dusts | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

| F8. Suspension | | | | |
|---|---|---|---|---|
| concentrates | a) | b) | c) | d) |
| active ingredient | 3% | 10% | 25% | 50% |
| ethylene glycol | 5% | 5% | 5% | 5% |
| nonylphenol polyglycol | — | 1% | 2% | — |
| ether (15 mol of ethylene | | | | |
| oxide) | | | | |
| sodium lignosulfonate | 3% | 3% | 4% | 5% |
| carboxymethylcellulose | 1% | 1% | 1% | 1% |
| 37% aqueous formaldehyde | 0.2% | 0.2% | 0.2% | 0.2% |
| solution | | | | |
| silicone oil emulsion | 0.8% | 0.8% | 0.8% | 0.8% |
| water | 87% | 79% | 62% | 38% |

The finely ground active ingredient is intimately mixed with the adjuvants, giving a suspension concentrate from which suspensions of any desired concentration can be obtained by dilution with water.

Compounds of the invention (as well as mixtures and/or compositions or formulations containing the same) find utility as herbicides, and may thus be employed in methods of controlling plant growth. Such methods involve applying to the plants or to the locus thereof an herbicidally effective amount of said compound, or composition comprising the same (or mixture as described hereinafter). The invention thus also relates to a method of inhibiting plant growth which comprises applying to the plants or to the locus thereof a herbicidally effective amount of a compound of formula (I), composition, or mixture of the invention. In particular the invention provides a method of controlling weeds in crops of useful plants, which comprising applying to said weeds or the locus of said weeds, or to said crop of useful plants, a compound of formula I or a composition or mixture containing the same.

The term “locus” as used herein includes not only areas where weeds may already be growing, but also areas where weeds have yet to emerge, and also to areas under cultivation with respect to crops of useful plants. Areas under cultivation include land on which the crop plants are already growing and land intended for cultivation with such crop plants.

A compound, composition, and/or mixture of the invention may be used in a pre-emergence application and/or in a post-emergence application in order to mediate its effect.

Crops of useful plants in which compounds of formula (I), as well as formulations and/or mixtures containing the same, may be used according to the invention include perennial crops, such as citrus fruit, grapevines, nuts, oil palms, olives, pome fruit, stone fruit and rubber, and annual arable crops, such as cereals, for example barley and wheat, cotton, oilseed rape, maize, rice, soy beans, sugar beet, sugar cane, sunflowers, ornamentals and vegetables, especially cereals and maize.

Compounds of formula (I), formulations and/or mixtures containing the same may also be used on turf, pasture, rangeland, rights of way etc. In particular they may used on golf-courses, lawns, parks, sports-fields, race-courses and the like.

Crops are to be understood as also including those crops which have been rendered tolerant to herbicides or classes of herbicides (e.g. ALS-, GS-, EPSPS-, PPO- and HPPD-inhibitors and synthetic auxins) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®, as well as corn, soybean and cotton that have been engineered to be resistant to Dicamba, phenoxypropionic acids, pyridyloxyacetic acids and/or picolinate auxins.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood as being those which are obtained by conventional methods of breeding or genetic engineering and contain so-called output traits (e.g. improved storage stability, higher nutritional value and improved flavour).

The term “weeds” as used herein means any undesired plant, and thus includes not only agronomically important weeds as described below, but also volunteer crop plants.

Compounds of formula (I) may be used against a large number of agronomically important weeds. The weeds that may be controlled include both monocotyledonous and dicotyledonous weeds, such as, for example, Alisma spp, Leptochloa chinensis, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum, Rottboellia, Cyperus, Abutilon, Sida, Xanthium, Amaranthus, Ambrosia, Brachiaria, Bidens, Chenopodium, Ipomoea, Chrysanthemum, Galium, Viola, Veronica, and Ischaemum spp.

The rates of application of compounds of formula (I) may vary within wide limits and depend on the nature of the soil, the method of application (pre- or post-emergence; seed dressing; application to the seed furrow; no tillage application etc.), the crop plant, or weed to be controlled, the prevailing climatic conditions, and other factors governed by the method of application, the time of application and the target crop. The compounds of formula I according to the invention are generally applied at a rate of from 10 to 2000 g/ha, especially from 50 to 1000 g/ha.

Any method of application to weeds/crop of useful plant, or locus thereof, which is routinely used in agriculture may be used, for example application by spray or broadcast method typically after suitable dilution of a compound of formula (I) (whether said compound is formulated and/or in combination with one or more further active ingredients and/or safeners, as described herein).

The compounds of formula (I) according to the invention can also be used in combination with other active ingredients, e.g. other herbicides, and/or insecticides, and/or acaricides, and/or nematocides, and/or molluscicides, and/or fungicides, and/or plant growth regulators. Such mixtures, and the use of such mixtures to control weeds and/or undesired plant growth form yet further aspects of the invention. For the avoidance of doubt, mixtures of invention also include mixtures of two or more different compounds of formula (I).

Where a compound of formula (I) is combined with at least one additional herbicide, the following mixtures of the compound of formula (I) are particularly preferred. Compound of formula (I)+acetochlor, compound of formula (I)+acifluorfen, compound of formula (I)+acifluorfen-sodium, compound of formula (I)+aclonifen, compound of formula (I)+acrolein, compound of formula (I)+alachlor, compound of formula (I)+alloxydim, compound of formula (I)+allyl alcohol, compound of formula (I)+ametryn, compound of formula (I)+amicarbazone, compound of formula (I)+amidosulfuron, compound of formula (I)+aminopyralid, compound of formula (I)+aminocyclopyrachlor, compound of formula (I)+amitrole, compound of formula (I)+ammonium sulfamate, compound of formula (I)+anilofos, compound of formula (I)+asulam, compound of formula (I)+atrazine, formula (I)+aviglycine, formula (I)+azafenidin, compound of formula (I)+azimsulfuron, compound of formula (I)+BCPC, compound of formula (I)+beflubutamid, compound of formula (I)+benazolin, formula (I)+bencarbazone, compound of formula (I)+benfluralin, compound of formula (I)+benfuresate, compound of formula (I)+bensulfuron, compound of formula (I)+bensulfuron-methyl, compound of formula (I)+bensulide, compound of formula (I)+bentazone, compound of formula (I)+benzfendizone, compound of formula (I)+benzobicyclon, compound of formula (I)+benzofenap, compound of formula (I)+bifenox, compound of formula (I)+bilanafos, compound of formula (I)+bispyribac, compound of formula (I)+bispyribac-sodium, compound of formula (I)+borax, compound of formula (I)+bromacil, compound of formula (I)+bromobutide, formula (I)+bromophenoxim, compound of formula (I)+bromoxynil, compound of formula (I)+butachlor, compound of formula (I)+butafenacil, compound of formula (I)+butamifos, compound of formula (I)+butralin, compound of formula (I)+butroxydim, compound of formula (I)+butylate, compound of formula (I)+cacodylic acid, compound of formula (I)+calcium chlorate, compound of formula (I)+cafenstrole, compound of formula (I)+carbetamide, compound of formula (I)+carfentrazone, compound of formula (I)+carfentrazone-ethyl, compound of formula (I)+CDEA, compound of formula (I)+CEPC, compound of formula (I)+chlorflurenol, compound of formula (I)+chlorflurenol-methyl, compound of formula (I)+chloridazon, compound of formula (I)+chlorimuron, compound of formula (I)+chlorimuron-ethyl, compound of formula (I)+chloroacetic acid, compound of formula (I)+chlorotoluron, compound of formula (I)+chlorpropham, compound of formula (I)+chlorsulfuron, compound of formula (I)+chlorthal, compound of formula (I)+chlorthal-dimethyl, compound of formula (I)+cinidon-ethyl, compound of formula (I)+cinmethylin, compound of formula (I)+cinosulfuron, compound of formula (I)+cisanilide, compound of formula (I)+clethodim, compound of formula (I)+clodinafop, compound of formula (I)+clodinafop-propargyl, compound of formula (I)+clomazone, compound of formula (I)+clomeprop, compound of formula (I)+clopyralid, compound of formula (I)+cloransulam, compound of formula (I)+cloransulam-methyl, compound of formula (I)+CMA, compound of formula (I)+4-CPB, compound of formula (I)+CPMF, compound of formula (I)+4-CPP, compound of formula (I)+CPPC, compound of formula (I)+cresol, compound of formula (I)+cumyluron, compound of formula (I)+cyanamide, compound of formula (I)+cyanazine, compound of formula (I)+cycloate, compound of formula (I)+cyclosulfamuron, compound of formula (I)+cycloxydim, compound of formula (I)+cyhalofop, compound of formula (I)+cyhalofop-butyl, compound of formula (I)+2,4-D, compound of formula (I)+3,4-DA, compound of formula (I)+daimuron, compound of formula (I)+dalapon, compound of formula (I)+dazomet, compound of formula (I)+2,4-DB, compound of formula (I)+3,4-DB, compound of formula (I)+2,4-DEB, compound of formula (I)+desmedipham, formula (I)+desmetryn, compound of formula (I)+dicamba, compound of formula (I)+dichlobenil, compound of formula (I)+ortho-dichlorobenzene, compound of formula (I)+para-dichlorobenzene, compound of formula (I)+dichlorprop, compound of formula (I)+dichlorprop-P, compound of formula (I)+diclofop, compound of formula (I)+diclofop-methyl, compound of formula (I)+diclosulam, compound of formula (I)+difenzoquat, compound of formula (I)+difenzoquat metilsulfate, compound of formula (I)+diflufenican, compound of formula (I)+diflufenzopyr, compound of formula (I)+dimefuron, compound of formula (I)+dimepiperate, compound of formula (I)+dimethachlor, compound of formula (I)+dimethametryn, compound of formula (I)+dimethenamid, compound of formula (I)+dimethenamid-P, compound of formula (I)+dimethipin, compound of formula (I)+dimethylarsinic acid, compound of formula (I)+dinitramine, compound of formula (I)+dinoterb, compound of formula (I)+diphenamid, formula (I)+dipropetryn, compound of formula (I)+diquat, compound of formula (I)+diquat dibromide, compound of formula (I)+dithiopyr, compound of formula (I)+diuron, compound of formula (I)+DNOC, compound of formula (I)+3,4-DP, compound of formula (I)+DSMA, compound of formula (I)+EBEP, compound of formula (I)+endothal, compound of formula (I)+EPTC, compound of formula (I)+esprocarb, compound of formula (I)+ethalfluralin, compound of formula (I)+ethametsulfuron, compound of formula (I)+ethametsulfuron-methyl, formula (I)+ethephon, compound of formula (I)+ethofumesate, compound of formula (I)+ethoxyfen, compound of formula (I)+ethoxysulfuron, compound of formula (I)+etobenzanid, compound of formula (I)+fenoxaprop, compound of formula (I)+fenoxaprop-P, compound of formula (I)+fenoxaprop-ethyl, compound of formula (I)+fenoxaprop-P-ethyl, compound of formula (I)+fentrazamide, compound of formula (I)+ferrous sulfate, compound of formula (I)+flamprop-M, compound of formula (I)+flazasulfuron, compound of formula (I)+florasulam, compound of formula (I)+fluazifop, compound of formula (I)+fluazifop-butyl, compound of formula (I)+fluazifop-P, compound of formula (I)+fluazifop-P-butyl, formula (I)+fluazolate, compound of formula (I)+flucarbazone, compound of formula (I)+flucarbazone-sodium, compound of formula (I)+flucetosulfuron, compound of formula (I)+fluchloralin, compound of formula (I)+flufenacet, compound of formula (I)+flufenpyr, compound of formula (I)+flufenpyr-ethyl, formula (I)+flumetralin, compound of formula (I)+flumetsulam, compound of formula (I)+flumiclorac, compound of formula (I)+flumiclorac-pentyl, compound of formula (I)+flumioxazin, formula (I)+flumipropin, compound of formula (I)+fluometuron, compound of formula (I)+fluoroglycofen, compound of formula (I)+fluoroglycofen-ethyl, formula (I)+fluoxaprop, formula (I)+flupoxam, formula (I)+flupropacil, compound of formula (I)+flupropanate, compound of formula (I)+flupyrsulfuron, compound of formula (I)+flupyrsulfuron-methyl-sodium, compound of formula (I)+flurenol, compound of formula (I)+fluridone, compound of formula (I)+fluorochloridone, compound of formula (I)+fluoroxypyr, compound of formula (I)+flurtamone, compound of formula (I)+fluthiacet, compound of formula (I)+fluthiacet-methyl, compound of formula (I)+fomesafen, compound of formula (I)+foramsulfuron, compound of formula (I)+fosamine, compound of formula (I)+glufosinate, compound of formula (I)+glufosinate-ammonium, compound of formula (I)+glyphosate, compound of formula (I)+halosulfuron, compound of formula (I)+halosulfuron-methyl, compound of formula (I)+haloxyfop, compound of formula (I)+haloxyfop-P, compound of formula (I)+HC-252, compound of formula (I)+hexazinone, compound of formula (I)+imazamethabenz, compound of formula (I)+imazamethabenz-methyl, compound of formula (I)+imazamox, compound of formula (I)+imazapic, compound of formula (I)+imazapyr, compound of formula (I)+imazaquin, compound of formula (I)+imazethapyr, compound of formula (I)+imazosulfuron, compound of formula (I)+indanofan, compound of formula (I)+iodomethane, compound of formula (I)+iodosulfuron, compound of formula (I)+iodosulfuron-methyl-sodium, compound of formula (I)+ioxynil, compound of formula (I)+isoproturon, compound of formula (I)+isouron, compound of formula (I)+isoxaben, compound of formula (I)+isoxachlortole, compound of formula (I)+isoxaflutole, formula (I)+isoxapyrifop, compound of formula (I)+karbutilate, compound of formula (I)+lactofen, compound of formula (I)+lenacil, compound of formula (I)+linuron, compound of formula (I)+MAA, compound of formula (I)+MAMA, compound of formula (I)+MCPA, compound of formula (I)+MCPA-thioethyl, compound of formula (I)+MCPB, compound of formula (I)+mecoprop, compound of formula (I)+mecoprop-P, compound of formula (I)+mefenacet, compound of formula (I)+mefluidide, compound of formula (I)+mesosulfuron, compound of formula (I)+mesosulfuron-methyl, compound of formula (I)+mesotrione, compound of formula (I)+metam, compound of formula (I)+metamifop, compound of formula (I)+metamitron, compound of formula (I)+metazachlor, compound of formula (I)+methabenzthiazuron, formula (I)+methazole, compound of formula (I)+methylarsonic acid, compound of formula (I)+methyldymron, compound of formula (I)+methyl isothiocyanate, compound of formula (I)+metobenzuron, formula (I)+metobromuron, compound of formula (I)+metolachlor, compound of formula (I)+S-metolachlor, compound of formula (I)+metosulam, compound of formula (I)+metoxuron, compound of formula (I)+metribuzin, compound of formula (I)+metsulfuron, compound of formula (I)+metsulfuron-methyl, compound of formula (I)+MK-616, compound of formula (I)+molinate, compound of formula (I)+monolinuron, compound of formula (I)+MSMA, compound of formula (I)+naproanilide, compound of formula (I)+napropamide, compound of formula (I)+naptalam, formula (I)+NDA-402989, compound of formula (I)+neburon, compound of formula (I)+nicosulfuron, formula (I)+nipyraclofen, formula (I)+n-methyl glyphosate, compound of formula (I)+nonanoic acid, compound of formula (I)+norflurazon, compound of formula (I)+oleic acid (fatty acids), compound of formula (I)+orbencarb, compound of formula (I)+orthosulfamuron, compound of formula (I)+oryzalin, compound of formula (I)+oxadiargyl, compound of formula (I)+oxadiazon, compound of formula (I)+oxasulfuron, compound of formula (I)+oxaziclomefone, compound of formula (I)+oxyfluorfen, compound of formula (I)+paraquat, compound of formula (I)+paraquat dichloride, compound of formula (I)+pebulate, compound of formula (I)+pendimethalin, compound of formula (I)+penoxsulam, compound of formula (I)+pentachlorophenol, compound of formula (I)+pentanochlor, compound of formula (I)+pentoxazone, compound of formula (I)+pethoxamid, compound of formula (I)+petrolium oils, compound of formula (I)+phenmedipham, compound of formula (I)+phenmedipham-ethyl, compound of formula (I)+picloram, compound of formula (I)+picolinafen, compound of formula (I)+pinoxaden, compound of formula (I)+piperophos, compound of formula (I)+potassium arsenite, compound of formula (I)+potassium azide, compound of formula (I)+pretilachlor, compound of formula (I)+primisulfuron, compound of formula (I)+primisulfuron-methyl, compound of formula (I)+prodiamine, compound of formula (I)+profluazol, compound of formula (I)+profoxydim, formula (I)+prohexadione-calcium, compound of formula (I)+prometon, compound of formula (I)+prometryn, compound of formula (I)+propachlor, compound of formula (I)+propanil, compound of formula (I)+propaquizafop, compound of formula (I)+propazine, compound of formula (I)+propham, compound of formula (I)+propisochlor, compound of formula (I)+propoxycarbazone, compound of formula (I)+propoxycarbazone-sodium, compound of formula (I)+propyzamide, compound of formula (I)+prosulfocarb, compound of formula (I)+prosulfuron, compound of formula (I)+pyraclonil, compound of formula (I)+pyraflufen, compound of formula (I)+pyraflufen-ethyl, formula (I)+pyrasulfotole, compound of formula (I)+pyrazolynate, compound of formula (I)+pyrazosulfuron, compound of formula (I)+pyrazosulfuron-ethyl, compound of formula (I)+pyrazoxyfen, compound of formula (I)+pyribenzoxim, compound of formula (I)+pyributicarb, compound of formula (I)+pyridafol, compound of formula (I)+pyridate, compound of formula (I)+pyriftalid, compound of formula (I)+pyriminobac, compound of formula (I)+pyriminobac-methyl, compound of formula (I)+pyrimisulfan, compound of formula (I)+pyrithiobac, compound of formula (I)+pyrithiobac-sodium, formula (I)+pyroxasulfone, formula (I)+pyroxulam, compound of formula (I)+quinclorac, compound of formula (I)+quinmerac, compound of formula (I)+quinoclamine, compound of formula (I)+quizalofop, compound of formula (I)+quizalofop-P, compound of formula (I)+quizalofop-ethyl, compound of formula (I)+quizalofop-P-ethyl, compound of formula (I)+rimsulfuron, compound of formula (I)+sethoxydim, compound of formula (I)+siduron, compound of formula (I)+simazine, compound of formula (I)+simetryn, compound of formula (I)+SMA, compound of formula (I)+sodium arsenite, compound of formula (I)+sodium azide, compound of formula (I)+sodium chlorate, compound of formula (I)+sulcotrione, compound of formula (I)+sulfentrazone, compound of formula (I)+sulfometuron, compound of formula (I)+sulfometuron-methyl, compound of formula (I)+sulfosate, compound of formula (I)+sulfosulfuron, compound of formula (I)+sulfuric acid, compound of formula (I)+tar oils, compound of formula (I)+2,3,6-TBA, compound of formula (I)+TCA, compound of formula (I)+TCA-sodium, formula (I)+tebutam, compound of formula (I)+tebuthiuron, formula (I)+tefuryltrione, compound of formula 1+tembotrione, compound of formula (I)+tepraloxydim, compound of formula (I)+terbacil, compound of formula (I)+terbumeton, compound of formula (I)+terbuthylazine, compound of formula (I)+terbutryn, compound of formula (I)+thenylchlor, compound of formula (I)+thiazafluoron, compound of formula (I)+thiazopyr, compound of formula (I)+thifensulfuron, compound of formula (I)+thiencarbazone, compound of formula (I)+thifensulfuron-methyl, compound of formula (I)+thiobencarb, compound of formula (I)+tiocarbazil, compound of formula (I)+topramezone, compound of formula (I)+tralkoxydim, compound of formula (I)+tri-allate, compound of formula (I)+triasulfuron, compound of formula (I)+triaziflam, compound of formula (I)+tribenuron, compound of formula (I)+tribenuron-methyl, compound of formula (I)+tricamba, compound of formula (I)+triclopyr, compound of formula (I)+trietazine, compound of formula (I)+trifloxysulfuron, compound of formula (I)+trifloxysulfuron-sodium, compound of formula (I)+trifluralin, compound of formula (I)+triflusulfuron, compound of formula (I)+triflusulfuron-methyl, compound of formula (I)+trifop, compound of formula (I)+trifop-methyl, compound of formula (I)+trihydroxytriazine, compound of formula (I)+trinexapac-ethyl, compound of formula (I)+tritosulfuron, compound of formula (I)+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS RN 353292-31-6), compound of formula (I)+4-hydroxy-3-[[2-[(2-methoxyethoxy)methyl]-6-(trifluoromethyl)-3-pyridinyl]carbonyl]-bicyclo[3.2.1]oct-3-en-2-one (CAS RN 352010-68-5), and compound of formula (I)+4-hydroxy-3-[[2-(3-methoxypropyl)-6-(difluoromethyl)-3-pyridinyl]carbonyl]-bicyclo[3.2.1]oct-3-en-2-one.

Whilst two-way mixtures of a compound of formula (I) and another herbicide are explicitly disclosed above, the skilled man will appreciate that the invention extends to three-way, and further multiple combinations comprising the above two-way mixtures.

In preferred embodiments a compound of formula (I) is combined with an acetolactate synthase inhibitor, (e.g. one or more of florasulam, metsulfuron, thifensulfuron, tribenuron, triasulfuron, flucarbazone, flupyrsulfuron, iodosulfuron, mesosulfuron, propoxicarbazone, sulfosulfuron, pyroxsulam and tritosulfuron, as well as salts or esters thereof), a synthetic auxin herbicide (e.g. one or more of aminocyclopyrachlor, aminopyralid, clopyralid, 2,4-D, 2,4-DB, dicamba, dichlorprop, fluoroxypyr, MCPA, MCPB, mecopropand mecoprop-P), an ACCase-inhibiting herbicide (e.g. one or more of phenylpyrazolin; pinoxaden; an aryloxyphenoxypropionic herbicide such as clodinafop, cyhalofop, diclofop, fenoxaprop, fluazifop, haloxyfop, quizalofop, trifop and mixtures thereof, as well as the isomers thereof, for example, fenoxaprop-P, fluazifop-P, haloxyfop-P, quizalofop-P; and a cyclohexanedione herbicide such as alloxydim; butroxydim, clethodim, cycloxydim, profoxydim, sethoxydim, tepraloxydim and tralkoxydim, as well as salts or esters thereof), and/or an auxin transport inhibitor such as semicarbazone (e.g. diflufenzopyr, in particular the sodium salt) or phthalamate compound (e.g. naptalam).

Particularly preferred mixture partners for compounds of formula (I) are: florasulam, iodosulfuron-methyl-sodium, mesosulfuron-methyl, metsulfuron-methyl, thifensulfuron, triasulfuron, tribenuron-methyl or pyroxsulam; dicamba, fluoroxypyr, MCPA, mecoprop or mecoprop-P; clodinafop-propargyl, cyhalofop-butyl, diclofop-methyl, fenoxaprop-ethyl, fenoxaprop-P-ethyl, fluazifop-butyl, fluazifop-P-butyl, haloxyfop-methyl, haloxyfop-P-methyl, pinoxaden, propaquizafop, quizalofop-ethyl, quizalofop-P-ethyl, tralkoxydim, trifop-methyl, diflufenzopyr-Na, and naptalam.

For the avoidance of doubt, even if not explicitly stated above, the mixing partners of the compound of formula (I) may also be in the form of any suitable agrochemically acceptable ester or salt, as mentioned e.g. in The Pesticide Manual, Thirteenth Edition, British Crop Protection Council, 2003.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

The compounds of formula (I) according to the invention can also be used in combination with one or more safeners. Likewise, mixtures of a compound of formula (I) according to the invention with one or more further active ingredients, in particular with one or more further herbicides, can also be used in combination with one or more safeners. Suitable safeners for use in combination with compounds of formula (I) include AD 67 (MON 4660), benoxacor, cloquintocet-mexyl, cyometrinil and the corresponding (Z) isomer, cyprosulfamide (CAS RN 221667-31-8), dichlormid, fenchlorazole-ethyl, fenclorim, flurazole, fluxofenim, furilazole and the corresponding R isomer, isoxadifen-ethyl, mefenpyr-diethyl, oxabetrinil, naphthalic anhydride (CAS RN 81-84-5) and N-isopropyl-4-(2-methoxy-benzoylsulfamoyl)-benzamide (CAS RN 221668-34-4). Particularly preferred safeners for use in the invention are cloquintocet-mexyl, cyprosulfamide, fenchlorazole-ethyl and mefenpyr-diethyl. The safeners of the compound of formula (I) may also be in the form of esters or salts, as mentioned e.g. in The Pesticide Manual, 13^{th} Edition supra. The reference to cloquintocet-mexyl also applies to a lithium, sodium, potassium, calcium, magnesium, aluminium, iron, ammonium, quaternary ammonium, sulfonium or phosphonium salt thereof as disclosed in WO02/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

Preferred mixtures of a compound of formula (I) with further herbicides and safeners include: a compound of formula (I)+pinoxaden+cloquinctocet-mexyl, a compound of formula (I)+clodinafop+cloquintocet-mexyl, and a compound of formula (I)+clodinafop-propargyl+cloquintocet-mexyl.

Various aspects and embodiments of the present invention will now be illustrated in more detail by way of example. It will be appreciated that modification of detail may be made without departing from the scope of the invention.

For the avoidance of doubt, where a literary reference, patent application, or patent, is cited within the text of this application, the entire text of said citation is herein incorporated by reference.

### EXAMPLES

### Example 1

### Synthesis of 2-cyclopropyl-4,5-dichloro-6-methoxycarbonyl pyrimidine

### 1.1 Preparation of Cyclopropylcarboxamidine Hydrochloride Salt

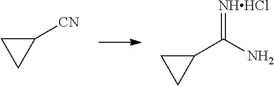

Hydrogen chloride gas was bubbled through a stirred solution of cyclopropylcarbonitrile (10.0 g, 0.15 mol) and methanol (6 ml, 0.15 mol) in dry ether (60 ml) at 0° C. for 2 hours. The reaction mixture was evaporated under reduced pressure and the residue dissolved in methanol (125 ml). The solution was added to an ice-cold mixture of methanol (125 ml) and liquid ammonia (15 ml) and the mixture stirred for 1 hour. The resulting clear solution was evaporated to leave cyclopropylcarboxamidine hydrochloride salt as a white solid (12.0 g, 67%).

¹H nmr (400 MHz, d₆-DMSO) δ_{H} 8.75 (2H, s), 8.64 (2H, s), 1.81 (1H, quintet), 1.11 (4H, s) ppm.

### 1.2 Preparation of 2-cyclopropyl-4-hydroxypyrimidine-6-carboxylic acid

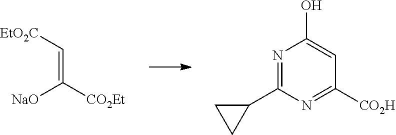

A solution of sodium hydroxide (2.85 g, 71.3 mmol) in water (3 ml) was added to a stirred solution of diethyl oxaloacetate sodium salt (8.7 g, 50 mmol) in water (50 ml) and the mixture stirred for 20 minutes. Cyclopropylcarboxamidine hydrochloride salt (5.0 g, 40 mmol) was added to the solution and the mixture was heated at 70° C. overnight, then cooled to ambient temperature and acidified to pH1 by the cautious addition of concentrated hydrochloric acid. The precipitate was isolated by filtration and dried to yield 2-cyclopropyl-4-hydroxypyrimidine-6-carboxylic acid (4.7 g, 63%).

¹H nmr (400 MHz, d₆-DMSO) δ_{H} 13.30 (1H, br s), 12.97 (1H, br s), 6.59 (1H, s), 1.94 (1H, quintet), 1.04 (4H, m) ppm.

### 1.3 Preparation of 5-chloro-2-cyclopropyl-4-hydroxypyrimidine-6-carboxylic acid

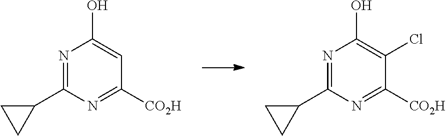

Aqueous sodium hypochlorite (470 ml) was added to a solution of 2-cyclopropyl-4-hydroxypyrimidine-6-carboxylic acid (68.7 g, 0.38 mol) in concentrated hydrochloric acid (280 ml) and water (412 ml), maintaining the temperature below 15° C. during the course of the addition. The reaction mixture was stirred for 12 hours at ambient temperature, then sodium metabisulphite (6.87 g) and sodium hydroxide (50% aqueous solution; 29.0 g) were added, maintaining the temperature below 15° C. during the addition. The precipitate was removed by filtration and dried to provide 5-chloro-2-cyclopropyl-4-hydroxypyrimidine-6-carboxylic acid (54 g, 66%).

¹H nmr (400 MHz, d₆-DMSO) δ_{H} 14.10 (1H, br s), 13.43 (1H, br s), 1.95 (1H, m), 1.08 (2H, m), 1.04 (2H, m) ppm.

### 1.4 Preparation of 2-cyclopropyl-4,5-dichloropyrimidine-6-carboxylic acid

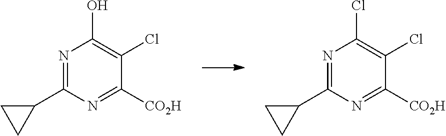

A mixture of 5-chloro-2-cyclopropyl-4-hydroxypyrimidine-6-carboxylic acid (10.0 g, 40 mmol) and phosphorus oxychloride (21.5 ml) was heated at reflux for 12 hours. The mixture was allowed to cool to ambient temperature and then added carefully to iced water and the resulting mixture extracted with ether. The combined ether layers were washed successively with water and brine, dried over sodium sulphate, filtered and the filtrate evaporated under reduced pressure to provide 2-cyclopropyl-4,5-dichloropyrimidine-6-carboxylic acid as a brown solid (7.0 g, 64%).

¹H nmr (400 MHz, d₆-DMSO) δ_{H} 3.37 (1H, br s), 2.24 (1H, m), 1.16 (2H, m), 1.08 (2H, m) ppm.

### 1.5 Preparation of 2-cyclopropyl-4,5-dichloro-6-methoxycarbonylpyrimidine

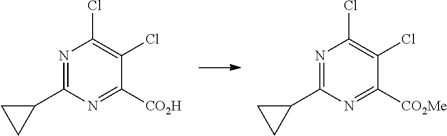

An excess of freshly prepared diazomethane was added to a solution of 2-cyclopropyl-4,5-dichloropyrimidine-6-carboxylic acid (50.0 g, 0.21 mol) in ether (1.5 l) at 0° C. After stirring for 15 minutes the reaction mixture was concentrated under reduced pressure to leave a brown oil. The residue was purified by column chromatography on silica using 10% ethyl acetate in hexane as eluent to provide 2-cyclopropyl-4,5-dichloro-6-methoxycarbonylpyrimidine (45.0 g, 85%) as a yellow oil, which solidified upon cooling. ¹H nmr (400 MHz, d₆-DMSO) δ_{H} 3.95 (3H, s), 2.25 (1H, quintet), 1.17 (2H, m), 1.03 (2H, m) ppm.

Further examples, prepared using the general method of Example 1, are given in Table 2 below.

TABLE 2
Compounds made according to the general method described in Example
1 above. Characteristic data is ¹H nmr data (400 MHz, CDCl₃) δ_{H} ppm

| Name | Structure | Characteristic data |
|---|---|---|
| 2-(4-Chloro-2-fluoro-3- methoxyphenyl)-4,5-dichloro-6- methoxycarbonylpyrimidine | 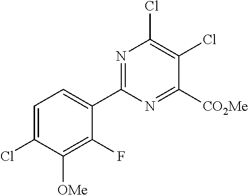 | 7.78 (1H, dd), 7.26 (1H, dd), 4.05 (3H, s), 4.01 (3H, s) |

### Example 2

### Synthesis of 4-amino-5-chloro-2-cyclopropyl-6-methoxycarbonyl pyrimidine

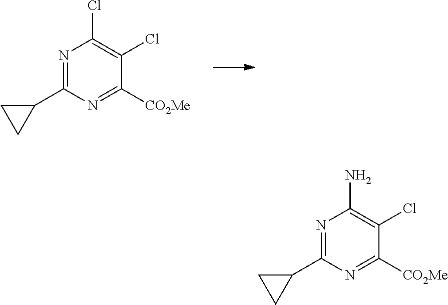

A mixture of 2-cyclopropyl-4,5-dichloro-6-methoxycarbonylpyrimidine (prepared as described in Example 1) (2.5 g, 10 mmol), ammonia (7M solution in methanol; 6 ml, 42 mmol) and methanol (8.3 ml) was heated in a microwave reactor at 120° C. for 20 minutes, then allowed to cool to ambient temperature. The solvent was evaporated under reduced pressure and the residue recrystallised from methanol to provide a white solid. The solid was dissolved in chloroform and the resulting solution washed with water and brine, dried over magnesium sulphate, filtered and the filtrate evaporated under reduced pressure to provide 4-amino-5-chloro-2-cyclopropyl-6-methoxycarbonylpyrimidine as a white solid (0.77 g, 38%).

¹H nm r (400 MHz, CDCl₃) δ_{H} 5.40 (2H, br s), 4.00 (3H, s), 2.10 (1H, m), 1.10 (4H, m) ppm.

### Example 3

### Synthesis of 5-chloro-2-cyclopropyl-4-dimethylamino-6-methoxycarbonylpyrimidine

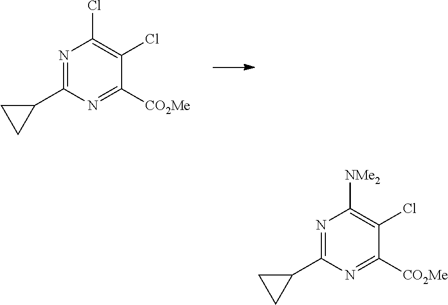

A mixture of 2-cyclopropyl-4,5-dichloro-6-methoxycarbonylpyrimidine (prepared as described in example 1) (0.5 g, 2.0 mmol), dimethylamine hydrochloride (325 mg, 4.0 mmol), triethylamine (0.55 ml, 4.0 mmol) and dichloromethane (7 ml) was stirred at ambient temperature for 4 hours. Ethyl acetate was added and the solution washed with brine, dried over magnesium sulphate, filtered and the filtrate evaporated under reduced pressure to provide 5-chloro-2-cyclopropyl-4-dimethylamino-6-methoxycarbonyl-pyrimidine as a white solid (0.46 g, 90%).

¹H nm r (400 MHz, CDCl₃) δ_{H} 4.00 (3H, s), 3.20 (6H, s), 2.10 (1H, m), 1.00 (4H, m) ppm.

### Example 4

### synthesis of 4-amino-2,5-dichloro-6-methoxycarbonylpyrimidine

### 4.1 Preparation of 2,4-dihydroxy-6-methoxycarbonylpyrimidine (methyl orotate)

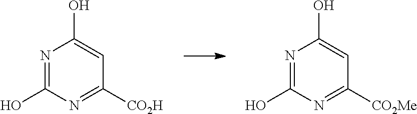

Thionyl chloride (500 ml), pyridine (2.5 ml) and a few drops of dimethylformamide were added to orotic acid monohydrate (78 g, 0.44 mol). The reaction mixture was stirred at RT for 5 days and then heated under reflux for an additional 14 hours. After cooling the solid material was allowed to settle and the supernatant decanted. The solid residue was washed with hexane and dried. Methanol (700 ml) was added dropwise with agitation to the solid. Once the rate of the gas formation slowed, the mixture was heated at reflux overnight and then cooled to 4-5° C. The solid was removed by filtration and washed with methanol and ether to provide methyl orotate (73 g, 97%).

¹H nmr (400 MHz, d₆-DMSO) δ_{H} 11.41 (1H, s), 11.26 (1H, s), 6.04 (1H, s), 3.84 (3H, s) ppm.

### 4.2 Preparation of 5-chloro-2,4-dihydroxy-6-methoxycarbonylpyrimidine

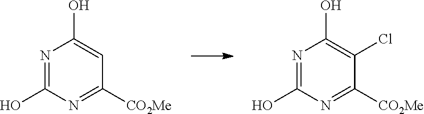

A catalytic quantity of ferric chloride was added to a solution of methyl orotate (34 g, 0.20 mol) in acetic anhydride (5% solution in glacial acetic acid, 500 ml). The mixture was heated to 90-95° C. and sulphuryl chloride (54 g, 0.40 mol) was added dropwise. After the addition was complete, the solution was slowly brought to reflux with agitation and heating was continued overnight. The solution was cooled to 18° C. and the solid was removed by filtration. The solid was washed with acetic acid and then with water, and dried to give 5-chloro-2,4-dihydroxy-6-methoxycarbonylpyrimidine (36.0 g, 89%).

¹H nmr (400 MHz, d₆-DMSO) δ_{H} 11.86 (1H, s), 11.62 (1H, s), 3.88 (3H, s) ppm.

### 4.3 Preparation of 6-methoxycarbonyl-2,4,5-trichloropyrimidine

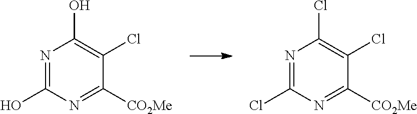

Phosphorus oxychloride (993 ml) was added to 5-chloro-2,4-dihydroxy-6-methoxycarbonylpyrimidine (30.0 g, 0.146 mol) at 10° C. and the resulting solution cooled to 0° C. N,N-Diethyl aniline (30.9 ml, 0.193 mol) was added dropwise to the stirred solution. After the addition was complete, the reaction mixture was allowed to warm slowly to ambient temperature and was then heated at reflux overnight. The resulting solution was cooled and concentrated under reduced pressure. The residue was poured onto crushed ice (600 g) and extracted with cold ether. The ether extracts were washed with brine, dried over sodium sulphate, filtered and evaporated under reduced pressure to give a light brown solid. This was triturated with warm hexane to yield 6-methoxycarbonyl-2,4,5-trichloropyrimidine (28 g, 82%).

¹H nmr (400 MHz, CDCl₃) δ_{H} 4.02 (3H, s) ppm.

### 4.4 Preparation of 4-amino-2,5-dichloro-6-methoxycarbonylpyrimidine

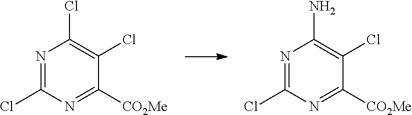

Aqueous ammonia (30% solution; 8.0 ml, 0.42 mol) was added dropwise to a stirred solution of 6-methoxycarbonyl-2,4,5-trichloropyrimidine (20.0 g, 0.083 mol) in THF (1000 ml) at 0° C. The reaction mixture was stirred at 0° C. for 1 hour and then filtered. The filtrate was evaporated under reduced pressure to give a white solid that was washed with twice with hexane and dried under vacuum to provide 4-amino-2,5-dichloro-6-methoxycarbonylpyrimidine (15.0 g, 82%).

¹H nmr (400 MHz, d₆-DMSO) δ_{H} 8.57 (1H, br s), 7.94 (1H, br s), 3.88 (3H, s) ppm.

### Example 5

### Synthesis of 4-amino-5-chloro-6-methoxycarbonyl-2-(4-trifluoro-methylphenyl)-pyrimidine

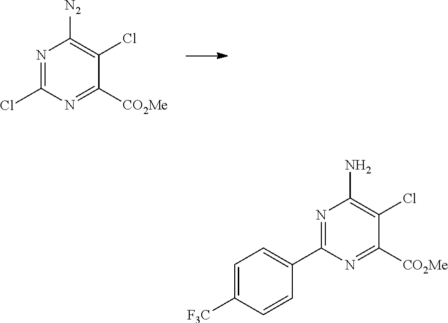

4-Trifluoromethylphenyl boronic acid (190 mg, 1.0 mmol), 4-amino-2,5-dichloro-6-methoxycarbonylpyrimidine (prepared as described in example 4) (221 mg, 1.0 mmol), caesium fluoride (302 mg, 2.0 mmol) and [1,1′-bis(diphenylphosphino)-ferrocene] dichloropalladium (II) complex with dichloromethane (1:1) (82 mg, 0.10 mmol) were placed in a vial. The vial was evacuated and backfilled with nitrogen before adding dimethoxyethane (2 ml) and water (2 ml). The reaction mixture was heated in a microwave reactor at 140° C. for 20 minutes, then allowed to cool and filtered through a plug of silica, washing with ethyl acetate. The filtrate was evaporated under reduced pressure and the crude product purified by chromatography on silica using a gradient of hexane/ethyl acetate (100:0 to 3:2) as eluent to provide 4-amino-5-chloro-6-methoxycarbonyl-2-(4-trifluoromethylphenyl)-pyrimidine as a pale yellow solid (102 mg, 31%).

M.p. 204-205° C.; ¹H nm r (400 MHz, CDCl₃) δ_{H} 8.50 (2H, d), 7.70 (2H, d), 5.60 (2H, br s), 4.00 (3H, s) ppm.

Further compounds, prepared using this general method, are listed in Table 3 below.

TABLE 3
Compounds made according to the general method described in Example 5
above. Characteristic data provided is either melting point (° C.) and/or
1H-nmr data (400 MHz, CDCl₃) δ_{H} ppm

| | | Characteristic |
|---|---|---|
| Name | Structure | data |
| 4-Amino-5- chloro-2-(4- chloro- phenyl)- 6-methoxy- carbonyl- pyrimidine | 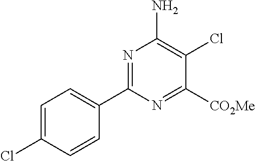 | 8.30 (2H, d), 7.4 (2H, d), 5.5 (2H, br s), 4.00 (3H, s) |
| 4-Amino-5- chloro-2-(4- chloro-2- fluoro-3- methoxy- phenyl)-6- methoxy- carbonyl- pyrimidine | 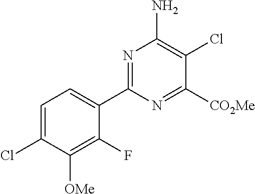 | 141-142 |
| 4-Amino-5- chloro-2-(4- chloro-3- dimethyl- amino-2- fluoro- phenyl)- 6-methoxy- carbonyl- pyrimidine | 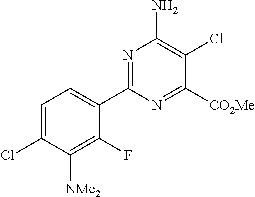 | 7.60 (1H, dd), 7.20 (1H, dd), 5.70 (2H, br s), 4.00 (3H, s), 2.9 (6H, s) |

### Example 6

### Synthesis of 4-amino-2-cyclopropyl-6-methoxycarbonyl-4-methylpyrimidine (compound 2-41)

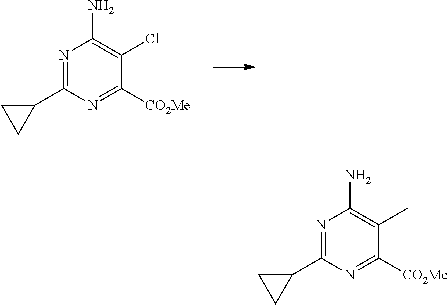

A mixture of methyl boronic acid (45 mg, 0.75 mmol), 4-amino-2,5-dichloro-6-methoxycarbonylpyrimidine (prepared as described in example 4) (114 mg, 0.50 mmol), palladium acetate (11 mg, 0.05 mmol), sodium 2-(dicyclohexylphosphino)-2′,6′-dimethoxybiphenyl-3′-sulphonate (38 mg, 0.075 mmol), potassium phosphate (212 mg, 1 mmol), toluene (2 ml) and water (0.4 ml) was heated in a microwave reactor at 110° C. for 40 minutes. The reaction mixture was allowed to cool and the solvent evaporated under reduced pressure. The residue was purified by chromatography on silica with 40% ethyl acetate in hexane as eluent to provide 4-amino-2-cyclopropyl-6-methoxycarbonyl-4-methylpyrimidine as an orange solid (27 mg, 26%).

M.p. 152-153° C.; ¹H nmr (400 MHz, CDCl₃) δ_{H} 4.92 (2H, br s), 3.95 (3H, s), 2.18 (3H, s), 2.07 (1H, m), 1.03 (2H, m), 0.95 (2H, m) ppm.

Further compounds, prepared using this general method, are listed in Table 4 below.

TABLE 4
Compounds made according to the general method described in Example 6
above. Characteristic data provided is either melting point (° C.) and/or 1H-
nmr data (400 MHz, CDCl₃) δ_{H} ppm

| Compound | | | |
|---|---|---|---|
| Number | Name | Structure | Characteristic data |
| 5-41 | 4-Amino-2-(4- chlorophenyl)-6- methoxycarbonyl- 4- methylpyrimidine | 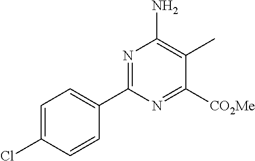 | 206-208 |
| 27-41 | 4-Amino-2-(4- chloro-2-fluoro-3- methoxyphenyl)- 6- methoxycarbonyl- 4- methylpyrimidine | 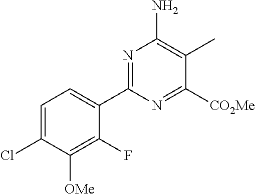 | 7.60 (1H, t), 7.20 (1H, d), 5.10 (2H, br s), 4.00 (3H, s), 3.95 (3H, s), 2.30 (3H, s) |
| 60-41 | 4-Amino-2-(2- fluoro-3-methoxy- 4-methylphenyl)- 6- methoxycarbonyl- 4- methylpyrimidine | 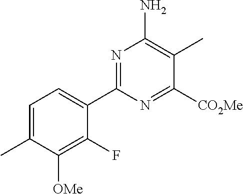 | 7.50 (1H, t), 7.00 (1H, d), 5.10 (2H, br s), 3.95 (3H, s), 3.90 (3H, s), 2.30 (3H, s), 2.25 (3H, s) |
| 33-41 | 4-Amino-2-(4- chloro-3- dimethylamino-2- fluorophenyl)-6- methoxycarbonyl- 4- methylpyrimidine | 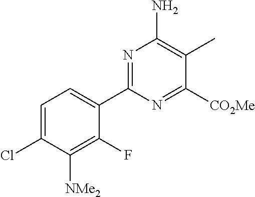 | 7.60 (1H, t), 7.20 (1H, dd), 5.20 (2H, br s), 3.90 (3H, s), 2.90 (6H, s), 2.30 (3H, s) |

### Example 7

### Synthesis of 4-amino-2-(4-chloro-3-dimethylamino-2-fluorophenyl)-5-cyclopropyl-6-methoxycarbonylpyrimidine (compound 33-58)

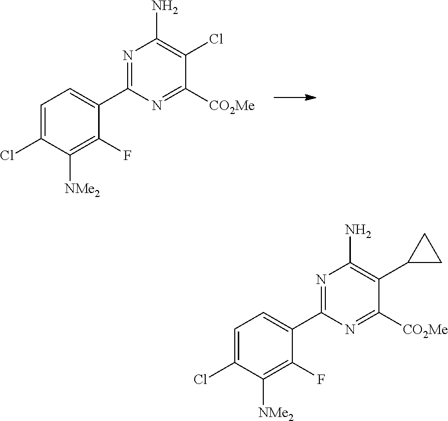

A mixture of 4-amino-5-chloro-2-(4-chloro-3-dimethylamino-2-fluorophenyl)-6-methoxycarbonylpyrimidine (prepared as described in example 5) (500 mg, 1.4 mmol), cyclopropyl boronic acid (144 mg, 1.68 mmol), palladium acetate (16 mg, 0.072 mmol), tricyclohexylphosphine tetrafluoroborate (52 mg, 0.14 mmol), potassium phosphate (890 mg, 4.2 mmol), toluene (9 ml) and water (1 ml) was purged with nitrogen, then heated in a microwave reactor at 160° C. for 30 minutes. The reaction mixture was allowed to cool, water added and the resulting mixture extracted with dichloromethane. The organic phase was washed with brine, dried over magnesium sulphate, filtered and evaporated under reduced pressure. The residue was purified by chromatography on silica using hexane/ethyl acetate (first 8:2, then 6:4) as eluent to provide 4-amino-2-(4-chloro-3-dimethylamino-2-fluorophenyl)-5-cyclopropyl-6-methoxycarbonylpyrimidine as an off-white solid (178 mg, 35%).

M.p. 156-158° C.; ¹H nmr (400 MHz, CDCl₃) δ_{H} 7.60 (1H, t), 7.20 (1H, dd), 5.50 (2H, br s), 4.00 (3H, s), 2.90 (6H, s), 1.70 (1H, m), 1.00 (2H, m), 0.60 (2H, m) ppm.

Further examples, prepared using this general method, are listed in Table 5 below.

TABLE 5
Compounds made according to the general method described in Example
7 above. Characteristic data is ¹H nmr data (400 MHz, CDCl₃) δ_{H} ppm

| Compound | | | |
|---|---|---|---|
| No. | Name | Structure | Characteristic data |
| 27-58 | 4-Amino-2-(4-chloro-2- fluoro-3- methoxyphenyl)-5- cyclopropyl-6- methoxycarbonyl- pyrimidine | 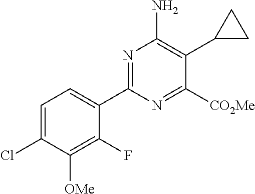 | 7.60 (1H, t), 7.20 (1H, dd), 5.50 (2H, br s), 4.00 (6H, s), 1.70 (1H, m), 1.00 (2H, m), 0.60 (2H, m) |

### Example 8

### Synthesis of 5-amino-2-(4-chloro-3-dimethylamino-2-fluorophenyl)-6-ethenyl-6-methoxycarbonylpyrimidine (compound 33-61)

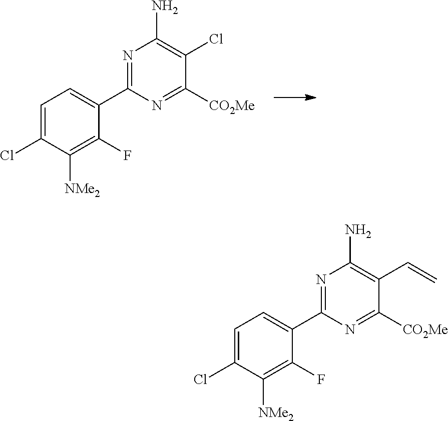

Vinyl boronic acid pinacol ester (86 μl, 0.51 mmol), 4-amino-5-chloro-2-(4-chloro-3-dimethylamino-2-fluorophenyl)-6-methoxycarbonyl-pyrimidine (prepared as described in example 5) (165 mg, 0.46 mmol), caesium fluoride (139 mg, 0.92 mmol) and [1,1′-bis(diphenylphosphino)-ferrocene] dichloropalladium (II) complex with dichloromethane (1:1) (38 mg, 46 μmol) were placed in a vial. The vial was evacuated and backfilled with nitrogen before adding dimethoxyethane (2 ml) and water (2 ml). The reaction mixture was heated in a microwave reactor at 140° C. for 20 minutes, then allowed to cool and filtered through a plug of silica, washing with ethyl acetate. The filtrate was evaporated under reduced pressure and the crude product purified by chromatography on silica using a gradient of hexane/ethyl acetate (100:0 to 8:2) as eluent to provide 5-amino-2-(4-chloro-3-dimethylamino-2-fluorophenyl)-5-ethenyl-6-methoxycarbonylpyrimidine as a solid (68 mg, 42%).

M.p. 136-137° C.; ¹H nmr (400 MHz, CDCl₃) δ_{H} 7.60 (1H, t), 7.20 (1H, dd), 6.80 (1H, dd), 5.70 (2H, m), 5.40 (2H, br s), 3.90 (3H, s), 2.90 (6H, s) ppm.

Further compounds, prepared using this general method, are listed in Table 6 below.

TABLE 6
Compounds made according to the general method described in Example 8
above. Characteristic data provided is either melting point (° C.) and/or 1H-
nmr data (400 MHz, CDCl₃) δ_{H} ppm

| Compound | | | |
|---|---|---|---|
| No. | Name | Structure | Characteristic data |
| 2-61 | 4-Amino-2- cyclopropyl-5- ethenyl-6- methoxycarbonyl- pyrimidine | 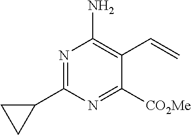 | 6.74 (1H, dd), 5.60 (1H, dd), 5.50 (1H, dd), 5.18 (2H, br s), 3.91 (3H, s), 2.10 (1H, m), 1.05 (2H, m), 0.97 (2H, m) |
| 5-61 | 4-Amino-2-(4- chlorophenyl)-5- ethenyl-6- methoxycarbonyl- pyrimidine | 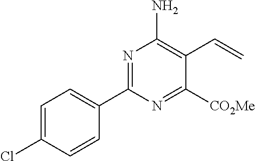 | 165-167 |
| 2-64 | 4-Amino-2- cyclopropyl-6- methoxycarbonyl- 5-(prop-1-en-2- yl)-pyrimidine | 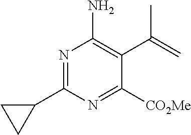 | 140-142 |
| 2-65 | 4-Amino-2- cyclopropyl-6- methoxycarbonyl- 5-(pent-1-enyl)- pyrimidine | 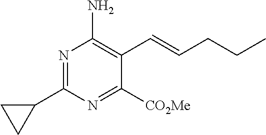 | 6.30 (1H, d), 5.90 (1H, dd), 5.10 (2H, br s), 3.90 (3H, s), 2.20 (2H, m), 2.10 (1H, m), 1.50 (2H, m), 1.10 (2H, m), 0.90 (5H, m) |
| 2-66 | 4-Amino-2- cyclopropyl-5-(2- cyclopropyl- ethenyl)-6- methoxycarbonyl- pyrimidine | 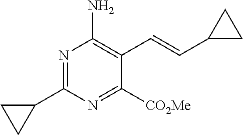 | 156-157 |
| 2-62 (Z isomer) | (Z)-4-Amino-2- cyclopropyl-6- methoxycarbonyl- 5-(prop-1-enyl)- pyrimidine | 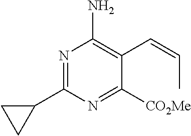 | 120-121 |
| 2-62 (E isomer) | (E)-4-Amino-2- cyclopropyl-6- methoxycarbonyl- 5-(prop-1-enyl)- pyrimidine | 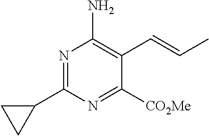 | 147-148 |
| 27-61 | 4-Amino-2-(4- chloro-2-fluoro-3- methoxyphenyl)- 5-ethenyl-6- methoxycarbonyl- pyrimidine | 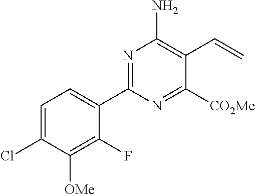 | 146-148 |
| 2-77 | 4-Amino-2- cyclopropyl-5- (3,3-dimethylbut- 1-ynyl)-6- methoxycarbonyl- pyrimidine | 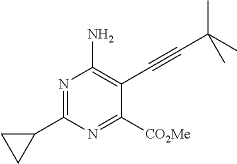 | 5.40 (2H, br s), 3.90 (3H, s), 2.10 (1H, m), 1.30 (9H, s), 1.10 (2H, m), 0.90 (2H, m) |
| 27-62 (E isomer) | (E)-4-Amino-2-(4- chloro-2-fluoro-3- methoxyphenyl)- 6- methoxycarbonyl- 5-(prop-1-enyl)- pyrimidine | 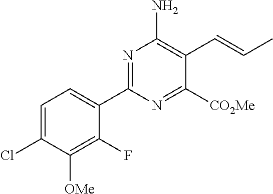 | 7.60 (1H, t), 7.20 (1H, d), 6.60 (1H, dd), 6.10 (1H, m), 5.30 (2H, br s), 4.00 (3H, s), 3.90 (3H, s), 1.90 (3H, d) |
| 31-61 | 4-Amino-5- ethenyl-6- methoxycarbonyl- 2-(4- trifluoromethyl- phenyl)-pyrimidine | 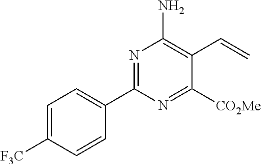 | 137-139 |
| 33-62 (E isomer) | (E)-4-Amino-2-(4- chloro-3- dimethylamino-2- fluorophenyl)-6- methoxycarbonyl- 5-(prop-1-enyl)- pyrimidine | 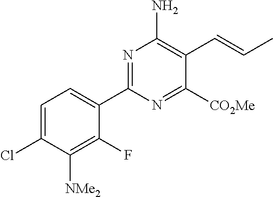 | 136-137 |
| 27-62 (Z isomer) | (Z)-4-Amino-2-(4- chloro-2-fluoro-3- methoxyphenyl)- 6- methoxycarbonyl- 5-(prop-1-enyl)- pyrimidine | 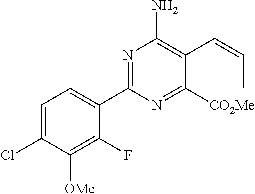 | 7.70 (1H, t), 7.30 (1H, dd), 6.40 (1H, dd), 6.10 (1H, m), 5.30 (2H, br s), 4.00 (3H, s), 3.90 (3H, s), 1.60 (3H, dd) |
| 2-221 | 2-Cyclopropyl-4- dimethylamino-6- methoxycarbonyl- 5- methylpyrimidine | 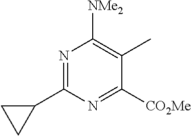 | 4.00 (3H, s), 3.00 (6H, s), 2.30 (3H, s), 2.10 (1H, m), 1.00 (4H, m) |
| 2-225 | 2-Cyclopropyl-4- dimethylamino-5- ethenyl-6- methoxycarbonyl- pyrimidine | 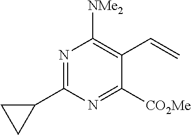 | 6.80 (1H, dd), 5.30 (2H, dd), 3.90 (3H, s), 3.10 (6H, s), 2.10 (1H, m), 1.00 (4H, m) |
| 27-63 | 4-Amino-2-(4- chloro-2-fluoro-3- methoxyphenyl)- 6- methoxycarbonyl- 5-(prop-1-en-2- yl)-pyrimidine | 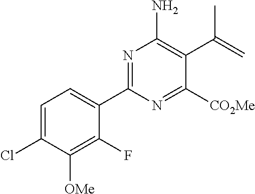 | 7.60 (1H, t), 7.20 (1H, dd), 5.40 (1H, s), 5.30 (2H, br s), 5.10 (1H, s), 4.00 (3H, s), 3.90 (3H, s), 2.10 (3H, s) |
| 27-69 | 4-Amino-2-(4- chloro-2-fluoro-3- methoxyphenyl)- 6- methoxycarbonyl- 5-(2-methylprop- 1-enyl)- pyrimidine | 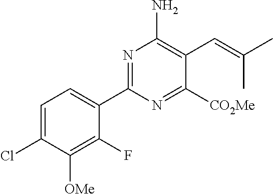 | 7.70 (1H, t), 7.20 (1H, dd), 6.10 (1H, s), 5.30 (2H, br s), 4.00 (3H, s), 3.90 (3H, s) 2.00 (3H, s), 1.60 (3H, s) |
| 2-69 | 4-Amino-2- cyclopropyl-6- methoxycarbonyl- 5-(2-methylprop- 1-enyl)- pyrimidine | 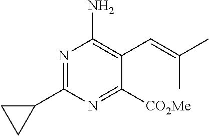 | 6.00 (1H, s), 5.00 (2H, br s), 3.90 (3H, s), 2.10 (1H, m), 1.9 (3H, s), 1.50 (3H, s), 1.10 (2H, m), 0.90 (2H, m). |
| 33-62 (Z isomer) | (Z)-4-Amino-2-(4- chloro-3- dimethylamino-2- fluorophenyl)-6- methoxycarbonyl- 5-(prop-1-enyl)- pyrimidine | 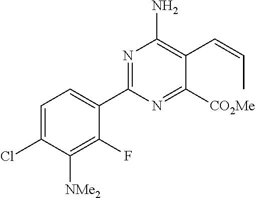 | 7.60 (1H, t), 7.20 (1H, d), 6.40 (1H, dd), 6.10 (1H, m), 5.40 (2H, m), 3.90 (3H, s), 2.90 (6H, s), 1.60 (3H, dd) |
| 33-64 | 4-Amino-2-(4- chloro-3- dimethylamino-2- fluorophenyl)-6- methoxycarbonyl- 5-(prop-1-en-2- yl)-pyrimidine | 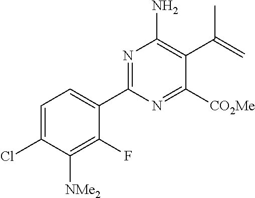 | 7.60 (1H, t), 7.20 (1H, dd), 5.40 (2H, br s), 5.40 (1H, m), 5.10 (1H, s), 3.90 (3H, s), 2.90 (6H, s), 2.10 (3H, s) |

### Example 9

### Synthesis of 4-amino-6-methoxycarbonyl-5-prop-1-enyl-2-(4-tri-fluoromethylphenyl)-pyrimidine (compound 31-62)

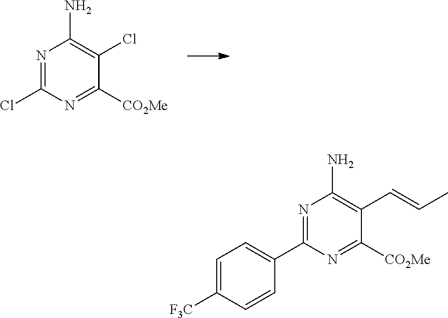

4-Trifluoromethylphenyl boronic acid (190 mg, 1.0 mmol), 4-amino-2,5-dichloro-6-methoxycarbonylpyrimidine (prepared as described in example 4) (221 mg, 1.0 mmol), caesium fluoride (302 mg, 2.0 mmol) and [1,1′-bis(diphenylphosphino)-ferrocene] dichloropalladium (II) complex with dichloromethane (1:1) (82 mg, 0.10 mmol) were placed in a vial. The vial was evacuated and backfilled with nitrogen before adding dimethoxyethane (2 ml) and water (2 ml). The reaction mixture was heated in a microwave reactor at 140° C. for 20 minutes, then allowed to cool and filtered through a plug of silica, washing with ethyl acetate. The filtrate was evaporated under reduced pressure and the residue dissolved in dimethoxyethane (2.5 ml) and water (2.5 ml). To this solution were added propenyl boronic acid (112 mg, 1.3 mmol), caesium fluoride (302 mg, 2.0 mmol) and [1,1′-bis(diphenylphosphino)-ferrocene] dichloropalladium (II) complex with dichloromethane (1:1) (82 mg, 0.10 mmol). The resulting mixture was heated in a microwave reactor at 140° C. for 20 minutes, then allowed to cool and filtered through a plug of silica, washing with ethyl acetate. The filtrate was washed with brine, dried over magnesium sulphate, filtered and evaporated under reduced pressure. The crude product was purified by chromatography on silica using a gradient of hexane/ethyl acetate (100:0 to 8:2) as eluent to provide 4-amino-6-methoxycarbonyl-5-prop-1-enyl-2-(4-trifluoromethylphenyl)-pyrimidine as an off-white solid (145 mg, 43%).

M.p. 123-125° C.; ¹H nmr (400 MHz, CDCl₃) δ_{H} 8.50 (2H, d), 7.70 (2H, d), 6.40 (1H, dd), 6.10 (1H, dd), 5.30 (2H, br s), 3.90 (3H, s), 1.90 (3H, dd) ppm.

Further compounds, prepared using this general method, are listed in Table 7 below.

TABLE 7
Compounds made according to the general method described in Example 9
above. Characteristic data provided is either melting point (° C.) and/or 1H-
nmr data (400 MHz, CDCl₃) δ_{H} ppm

| Compound | | | |
|---|---|---|---|
| No. | Name | Structure | Characteristic data |
| 31-64 | 4-Amino-6- methoxycarbonyl- 5-(prop-1-en-2- yl)-2-(4- trifluoromethyl- phenyl)-pyrimidine | 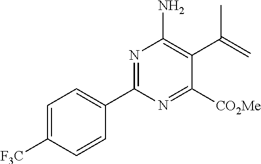 | 83-85 |
| 31-63 | 4-Amino-6- methoxycarbonyl- 5-(prop-1-en-3- yl)-2-(4- trifluoromethyl- phenyl)-pyrimidine | 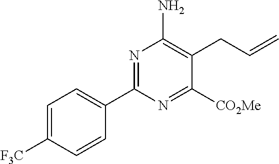 | 8.50 (2H, d), 7.70 (2H, d), 5.90 (1H, m), 5.20 (4H, m + br s), 4.00 (3H, s), 3.50 (2H, m) |
| 27-61 | 4-Amino-2-(4- chloro-2-fluoro-3- methoxyphenyl)- 5-ethenyl-6- methoxycarbonyl- pyrimidine | 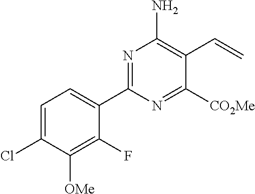 | 146-148 |

### Example 10

### Synthesis of 4-amino-5-ethenyl-6-methoxycarbonyl-2-methylthiopyrimidine (compound 62-61)

### 10.1 Preparation of 4-amino-5-chloro-6-methoxycarbonyl-2-methylthiopyrimidine

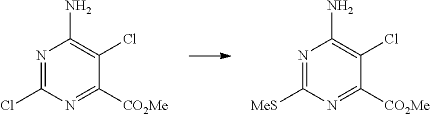

Sodium methanethiolate (3.0 g, 35 mmol) was added portionwise to a stirred solution of 4-amino-2,5-dichloro-6-methoxycarbonylpyrimidine (prepared as described in example 4) (4.4. g, 20 mmol) in methanol (100 ml) to give a pale yellow solution. The resulting mixture was stirred at reflux for 2 hours then allowed to cool for 2 hours, filtered and evaporated under reduced pressure. The residue was dissolved in water and ethyl acetate, the phases separated and the aqueous extracted with further ethyl acetate. The combined organic phases were washed with water and brine, dried over magnesium sulphate, filtered and evaporated under reduced pressure to provide 4-amino-5-chloro-6-methoxycarbonyl-2-methylthiopyrimidine as a yellow solid (2.2 g), which was used without further purification.

¹H nmr (400 MHz, CDCl₃) δ_{H} 5.55 (2H, br s), 3.95 (3H, s), 2.50 (3H, s) ppm.

### 10.2 Preparation of 4-amino-5-ethenyl-6-methoxycarbonyl-2-methylthiopyrimidine (compound 62-61)

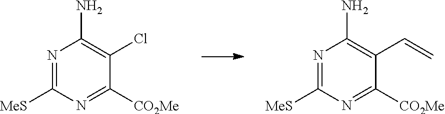

Water (2 ml) was added with stirring to a solution of 4-amino-5-chloro-6-methoxycarbonyl-2-methylthiopyrimidine (233 mg, 1.0 mmol) in dimethoxyethane (3 ml). The mixture was heated in a microwave reactor at 140° C. for 2 hours, then allowed to cool, diluted with ethyl acetate and washed with water and brine. The organic phase was dried over magnesium sulphate, filtered and evaporated under reduced pressure to provide a brown oil which was purified by chromatography on silica using hexane/ethyl acetate (4:1) as eluent to provide 4-amino-5-ethenyl-6-methoxycarbonyl-2-methylthiopyrimidine as a beige solid (120 mg, 50%).

¹H nmr (400 MHz, CDCl₃) δ_{H} 6.70 (1H, dd), 5.75 (2H, dd), 5.30 (2H, br s), 3.90 (3H, s), 2.50 (3H, s) ppm.

### Example 11

### Synthesis of 4-amino-5-ethenyl-6-methoxycarbonyl-2-(4-methylphenyl)-pyrimidine (compound 36-61)

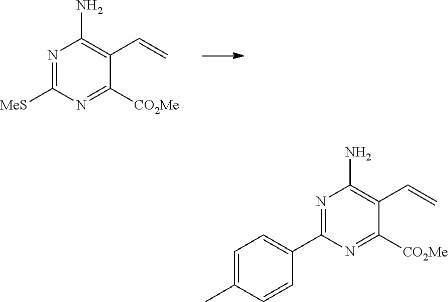

A solution of 4-amino-5-ethenyl-6-methoxycarbonyl-2-methylthiopyrimidine (prepared as described in example 10) (113 mg, 0.50 mmol), 4-methylphenylboronic acid (74 mg, 0.55 mmol), copper thiophene-2-carboxylate (125 mg, 0.65 mmol)), tri(2-furyl)phosphine (19 mg, 80 μmol) and tris(dibenzylideneacetone)dipalladium chloroform adduct (10 mg, 10 μmol) in tetrahydrofuran (3 ml) was heated in a microwave reactor at 100° C. for 30 minutes, then allowed to cool. Ether was added and the resulting solution washed with concentrated aqueous ammonia and brine, dried over magnesium sulphate, filtered and evaporated under reduced pressure to provide a brown solid (0.13 g). The crude product was purified by chromatography on silica using hexane/ethyl acetate (4:1) as eluent to provide 4-amino-5-ethenyl-6-methoxycarbonyl-2-(4-methylphenyl)-pyrimidine as a pale yellow solid (45 mg, 33%).

M.p. 122-123° C.; ¹H nmr (400 MHz, CDCl₃) δ_{H} 8.24 (2H, d), 7.22 (2H, d), 6.80 (1H, dd), 5.60 (2H, m), 5.35 (2H, br s), 3.93 (3H, s), 2.39 (3H, s) ppm.

Further compounds, prepared using this general method, are listed in Table 8 below.

TABLE 8
Compounds made according to the general method described in Example
11 above. Characteristic data provided is either melting point (° C.) and/or
1H-nmr data (400 MHz, CDCl₃) δ_{H} ppm

| Compound | | | |
|---|---|---|---|
| No. | Name | Structure | Characteristic data |
| 19-61 | 4-Amino-2-(4- chloro-3- fluorophenyl)-5- ethenyl-6- methoxycarbonyl- pyrimidine | 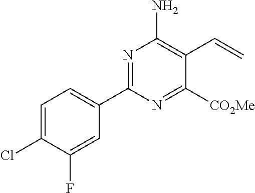 | 155-156 |
| 61-61 | 4-Amino-2-(3- chloro-5- fluorophenyl)-5- ethenyl-6- methoxycarbonyl- pyrimidine | 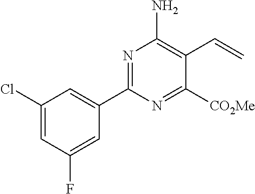 | 158-159 |
| 15-61 | 4-Amino-2-(4- chloro-2- fluorophenyl)-5- ethenyl-6- methoxycarbonyl- pyrimidine | 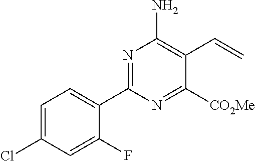 | 137-138 |
| 63-61 | 4-Amino-5- ethenyl-6- methoxycarbonyl- 2-(3,4,5- trifluorophenyl)- pyrimidine | 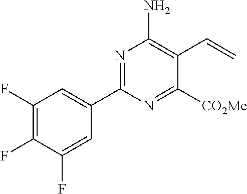 | 188-190 |
| 9-61 | 4-Amino-2-(3,4- dichlorophenyl)- 5-ethenyl-6- methoxycarbonyl- pyrimidine | 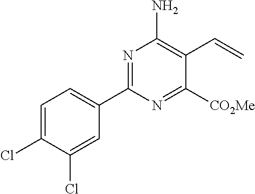 | 8.49 (1H, d), 8.22 (1H, dd), 7.50 (1H, d), 6.79 (1H, dd), 5.78 (1H, m), 5.64 (1H, dd), 5.40 (2H, br s), 3.98 (3H, s) |
| 14-61 | 4-Amino-2-(4- chloro-3- trifluoromethyl- phenyl)-5-ethenyl-6- methoxycarbonyl- pyrimidine | 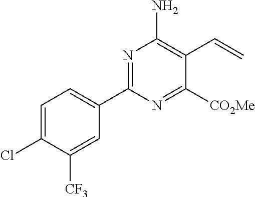 | 8.72 (1H, d), 8.49 (1H, dd), 7.56 (1H, d), 6.78 (1H, dd), 5.69 (1H, m), 5.65 (1H, dd), 5.42 (2H, br s), 3.97 (3H, s) |
| 6-61 | 4-Amino-2-(4- bromophenyl)-5- ethenyl-6- methoxycarbonyl- pyrimidine | 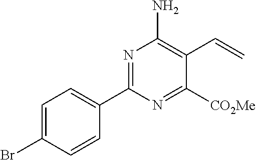 | 8.25 (2H, d), 7.56 (2H, d), 6.79 (1H, dd), 5.66 (1H, m), 5.63 (1H, dd), 5.38 (2H, br s), 3.96 (3H, s) |
| 41-61 | 4-Amino-5- ethenyl-2-(4- fluorophenyl)-6- methoxycarbonyl- pyrimidine | 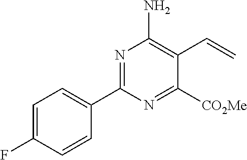 | 8.37 (2H, d), 7.11 (2H, m), 6.80 (1H, dd), 5.65 (1H, m), 5.62 (1H, dd), 5.37 (2H, br s), 3.96 (3H, s) |
| 44-61 | 4-Amino-2-(3- chloro-4- fluorophenyl)-5- ethenyl-6- methoxycarbonyl- pyrimidine | 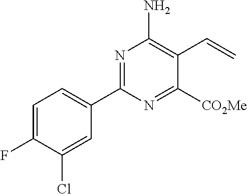 | 8.46 (1H, dd), 8.27 (1H, m), 7.19 (1H, t), 6.79 (1H, dd), 5.67 (1H, m), 5.63 (1H, dd), 5.39 (2H, br s), 3.96 (3H, s) |
| 43-61 | 4-Amino-5- ethenyl-2-(4- fluoro-3- trifluoromethyl- phenyl)-6- methoxycarbonyl- pyrimidine | 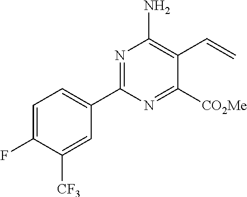 | 8.67 (1H, dd), 8.59 (1H, m), 7.25 (1H, t), 6.80 (1H, dd), 5.68 (1H, m), 5.64 (1H, dd), 5.41 (2H, br s), 3.97 (3H, s) |
| 45-61 | 4-Amino-5- ethenyl-2-(4- fluoro-3- methylphenyl)-6- methoxycarbonyl- pyrimidine | 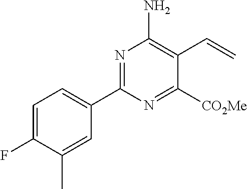 | 8.22 (1H, m), 8.18 (1H, m), 7.05 (1H, t), 6.79 (1H, dd), 5.66 (1H, m), 5.62 (1H, dd), 5.36 (2H, br s), 3.96 (3H, s), 2.34 (3H, s) |
| 42-61 | 4-Amino-5- ethenyl-2-(4- fluoro-3- methoxyphenyl)- 6- methoxycarbonyl- pyrimidine | 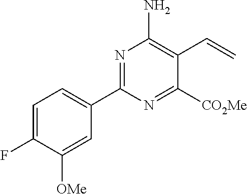 | 8.02 (1H, dd), 7.97 (1H, m), 7.13 (1H, dd), 6.79 (1H, dd), 5.67 (1H, m), 5.63 (1H, d), 5.37 (2H, br s), 4.00 (3H, s), 3.96 (3H, s) |
| 67-61 | 4-Amino-2-(3,4- difluorophenyl)-5- ethenyl-6- methoxycarbonyl- pyrimidine | 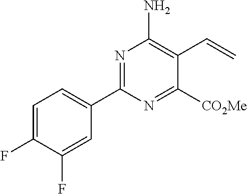 | 8.22 (1H, m), 8.15 (1H, m), 7.21 (1H, q), 6.80 (1H, dd), 5.67 (1H, d), 5.64 (1H, d), 5.38 (2H, br s), 3.97 (3H, s) |
| 68-61 | 4-Amino-2-(4- cyanophenyl)-5- ethenyl-6- methoxycarbonyl- pyrimidine | 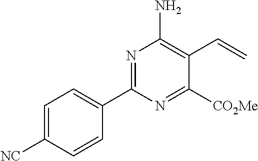 | 8.49 (2H, d), 7.74 (2H, d), 6.82 (1H, dd), 5.70 (1H, m), 5.66 (1H, dd), 5.41 (2H, br s), 3.97 (3H, s) |
| 69-61 | 4-Amino-5- ethenyl-6- methoxycarbonyl- 2-(4- methoxycarbonyl- phenyl)- pyrimidine | 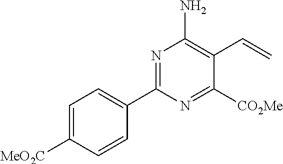 | 165-166 |
| 47-61 | 4-Amino-5- ethenyl-6- methoxycarbonyl- 2-(4- trifluoromethoxy- phenyl)-pyrimidine | 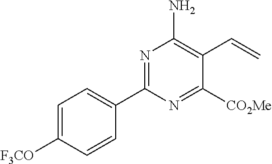 | 8.40 (2H, d), 7.27 (2H, d), 6.80 (1H, dd), 5.66 (1H, d), 5.63 (1H, d), 5.41 (2H, br s), 3.96 (3H, s) |
| 71-61 | 4-Amino-5- ethenyl-2-(3,4- ethylenedioxy- phenyl)-6- methoxycarbonyl- pyrimidine | 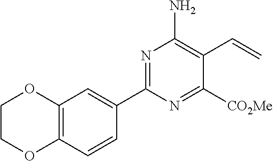 | 7.92 (1H, d), 7.88 (1H, dd), 6.90 (1H, d), 6.79 (1H, dd), 5.63 (1H, s), 5.59 (1H, d), 5.36 (2H, br s), 4.30 (4H, m), 3.94 (3H, s) |
| 30-61 | 4-Amino-2-(4- chloro-3,5- difluorophenyl)-5- ethenyl-6- methoxycarbonyl- pyrimidine | 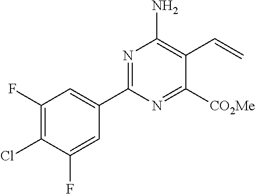 | 8.04 (2H, m), 6.79 (1H, dd), 5.66 (2H, m), 5.40 (2H, br s), 3.95 (3H, s) |
| 26-61 | 4-Amino-2-(4- chloro-3- methoxyphenyl)- 5-ethenyl-6- methoxycarbonyl- pyrimidine | 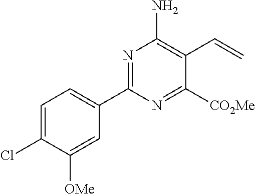 | 7.97 (1H, d), 7.95 (1H, dd), 7.42 (1H, d), 6.78 (1H, dd), 5.66 (1H, m), 5.63 (1H, dd), 5.39 (2H, br s), 4.03 (3H, s), 3.95 (3H, s) |
| 70-61 | 4-Amino-5- ethenyl-6- methoxycarbonyl- 2-(3,4- methylenedioxy- phenyl)-pyrimidine | 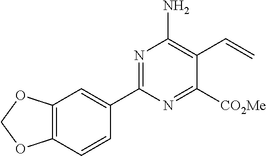 | 7.97 (1H, dd), 7.84 (1H, d), 6.88 (1H, d), 6.78 (1H, dd), 6.03 (2H, s), 5.84 (2H, br s), 5.67 (1H, dd), 5.63 (1H, dd), 3.96 (3H, s) |
| 72-61 | 4-Amino-5- ethenyl-6- methoxycarbonyl- 2-(4- methylthiophenyl)- pyrimidine | 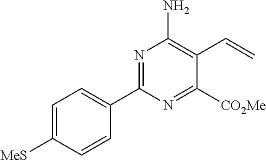 | 8.28 (2H, d), 7.27 (2H, d), 6.78 (1H, dd), 5.64 (1H, s), 5.59 (1H, d), 5.44 (2H, br s), 3.95 (3H, s), 2.53 (3H, s) |
| 73-61 | 4-Amino-5- ethenyl-6- methoxycarbonyl- 2-(2-naphthyl)- pyrimidine | 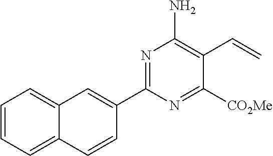 | 8.91 (1H, s), 8.47 (1H, d), 7.96 (1H, m), 7.88 (1H, d), 7.85 (1H, m), 7.49 (2H, m), 6.80 (1H, dd), 5.63 (2H, m), 5.45 (2H, br s), 3.98 (3H, s) |

### Example 12

### Synthesis of 4-amino-2-(4-chloro-2-fluoro-3-methoxyphenyl)-5-ethenylpyrimidine-6-carboxylic acid (compound 27-21)

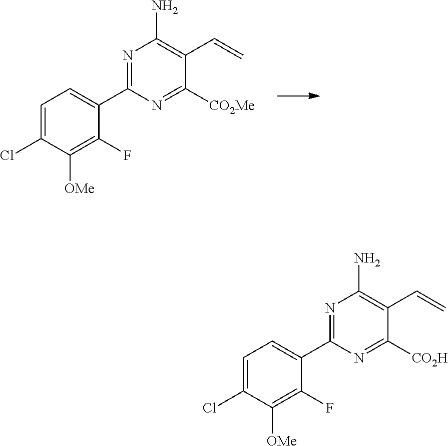

Sodium hydroxide (24 mg, 2 mmol) was added to a suspension of 4-amino-2-(4-chloro-2-fluoro-3-methoxyphenyl)-5-ethenyl-6-methoxycarbonylpyrimidine (prepared as described in example 8) (100 mg, 0.30 mmol) in tetrahydrofuran (10 ml) and water (6.5 ml) The reaction mixture was stirred for 3 hours at ambient temperature then acidified to pH 1-2 and washed with ethyl acetate. The aqueous phase was concentrated under reduced pressure to provide an off white solid which was purified using a FractionLynx automated hplc system to yield 4-amino-2-(4-chloro-2-fluoro-3-methoxyphenyl)-5-ethenylpyrimidine-6-carboxylic acid as a white solid (28 mg, 29%)

¹H nm r (400 MHz, d₆-DMSO) δ_{H} 7.50 (1H, m), 7.30 (1H, d), 6.50 (1H, m), 5.80 (1H, d), 5.30 (1H, d), 3.80 (3H, s) (amine and acid protons not observed).

Further examples, prepared using this general method, are listed in Table 9 below.

TABLE 9
Compounds made according to the general method described in
Example 12 above. Characteristic data is ¹H nmr data (400 MHz, d₆-
DMSO) δ_{H} ppm

| Compound | | | |
|---|---|---|---|
| No. | Name | Structure | Characteristic data |
| 33-21 | 4-Amino-2-(4-chloro-3- dimethylamino-2- fluorophenyl)-5- ethenylpyrimidine-6- carboxylic acid | 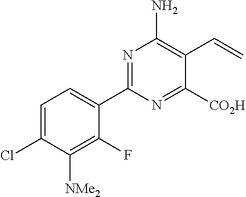 | 7.50 (1H, t), 7.20 (1H, t), 6.50 (3H, br s and t), 5.70 (1H, d), 5.20 (1H, d), 2.40 (6h, d) (acid proton not observed) |

### Example 13

### synthesis of 4-amino-2-(4-chloro-3-dimethylamino-2-fluorophenyl)-5-ethenyl-6-ethoxycarbonyl-pyrimidine (compound 33-101)

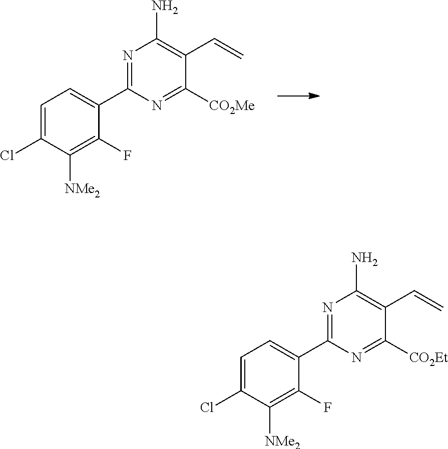

A solution of 4-amino-2-(4-chloro-3-dimethylamino-2-fluorophenyl)-5-ethenyl-6-methoxycarbonyl-pyrimidine (prepared by the method described in example 8) (200 mg, 0.57 mmol), 1-hydroxy-3-isothionato-1,1,3,3-tetrabutyl distannoxane (32 mg, 0.057 mmol), and ethanol (0.22 ml, 5.7 mmol) in toluene (8 ml) was heated at reflux for 3 hours. The reaction was allowed to cool and evaporated under reduced pressure and the residue purified by chromatography on silica using a hexane/ethyl acetate gradient (8:2 to 6:4) as eluent to provide 4-amino-2-(4-chloro-3-dimethylamino-2-fluorophenyl)-5-ethenyl-6-ethoxycarbonyl-pyrimidine as a white solid (193 mg, 93%).

¹H nmr (400 MHz, CDCl₃) δ_{H} 7.60 (1H, t), 7.20 (1H, dd), 6.80 (1H, dd), 5.70 (2H, dd), 5.50 (2H, br s), 4.40 (2H, q), 2.90 (6H, d), 1.40 (3H, t) ppm.

Further examples, prepared using this general method, are listed in Table 10 below.

TABLE 10
Compounds made according to the general method described in Example
13 above. Characteristic data is ¹H nmr data (400 MHz, CDCl₃) δ_{H} ppm

| Compound | | | Characteristic |
|---|---|---|---|
| No. | Name | Structure | data |
| 33-306 | 4-Amino-2-(4- chloro-3- dimethylamino-2- fluorophenyl)-5- ethenyl-6- isopropoxycarbon- yl-pyrimidine | 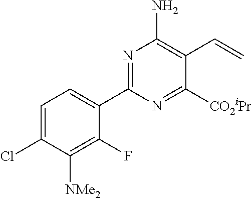 | 7.62 (1H, t), 7.21 (1H, dd), 6.72 (1H, dd), 5.67 (1H, m), 5.64 (1H, m), 5.36 (2H, br s), 5.28 (1H, septet), 2.90 (6H, d), 1.37 (6H, d) |
| 33-316 | 4-Amino-2-(4- chloro-3- dimethylamino-2- fluorophenyl)-5- ethenyl-6- ⁿpropoxycarbonyl- pyrimidine | 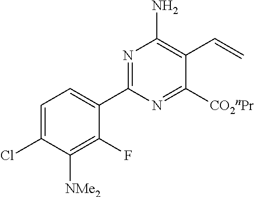 | 7.61 (1H, t), 7.22 (1H, dd), 6.76 (1H, dd), 5.68 (1H, s), 5.64 (1H, d), 5.42 (2H, br s), 4.31 (2H, t), 2.91 (6H, d), 1.80 (2H, sextet), 1.03 (3H, t) |
| 33-391 | 4-Amino-6- ⁿbutoxycarbonyl-2- (4-chloro-3- dimethylamino-2- fluorophenyl)-5- ethenyl-pyrimidine | 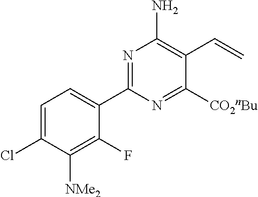 | 7.62 (1H, t), 7.22 (1H, dd), 6.77 (1H, dd), 5.68 (1H, s), 5.65 (1H, d), 5.39 (2H, br s), 4.36 (2H, t), 2.90 (6H, d), 1.76 (2H, m), 1.46 (2H, m), 0.96 (3H, t) |
| 33-326 | 4-Amino-2-(4- chloro-3- dimethylamino-2- fluorophenyl)-5- ethenyl-6-(2- methylpropoxy) carbonyl-pyrimidine | 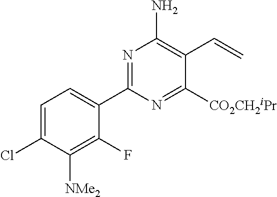 | 7.62 (1H, dd), 7.22 (1H, dd), 6.77 (1H, dd), 5.68 (1H, s), 5.64 (1H, d), 5.40 (2H, br s), 4.22 (1H, dd), 4.13 (1H, dd), 2.90 (6H, d), 1.93 (1H, m), 1.00 (3H, d), 0.92 (3H, t) |
| 33-392 | 4-Amino-2-(4- chloro-3- dimethylamino-2- fluorophenyl)-5- ethenyl-6- ⁿoctyloxycarbonyl- pyrimidine | 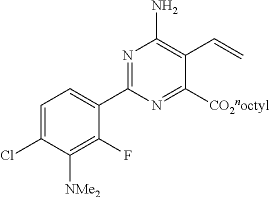 | 7.61 (1H, dd), 7.22 (1H, dd), 6.77 (1H, dd), 5.78 (1H, s), 5.64 (1H, d), 5.40 (2H, br s), 5.35 (2H, t), 2.90 (6H, d), 1.76 (2H, quintet), 1.43 (2H, m), 1.30 (8H, m), 0.88 (3H, m) |
| 33-393 | 4-Amino-2-(4- chloro-3- dimethylamino-2- fluorophenyl)-5- ethenyl-6-(2- ethylhexyloxy) carbonyl-pyrimidine | 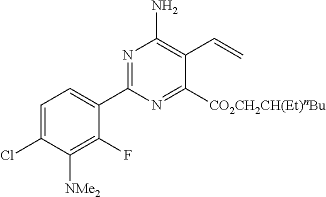 | 7.63 (1H, t), 7.21 (1H, dd), 6.76 (1H, dd), 5.68 (1H, m), 5.64 (1H, m), 5.38 (2H, br s), 4.26 (2H, m), 2.90 (6H, d), 1.72 (1H, m), 1.37 (8H, m), 1.27 (3H, t), 0.92 (3H, m) |
| 33-336 | 4-Amino-2-(4- chloro-3- dimethylamino-2- fluorophenyl)-5- ethenyl-6- phenylmethoxy- carbonyl-pyrimidine | 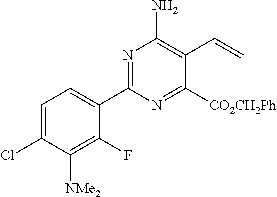 | 7.61 (1H, dd), 7.45 (2H, m), 7.36 (3H, m), 7.21 (1H, dd), 6.70 (1H, dd), 5.60 (1H, d), 5.56 (1H, s), 5.39 (4H, br s), 2.91 (6H, d) |
| 33-394 | 4-Amino-2-(4- chloro-3- dimethylamino-2- fluorophenyl)-5- ethenyl-6-(1- phenyl- ethoxy)carbonyl- pyrimidine | 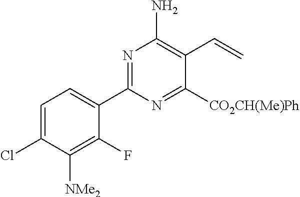 | 7.65 (1H, t), 7.45 (2H, m), 7.35 (3H, m), 7.22 (1H, dd), 6.64 (1H, dd), 6.14(1H, q), 5.59 (1H, d), 5.53(1H, dd), 5.38 (2H, br s), 2.91 (6H, d), 1.69 (3H, d) |
| 33-395 | 4-Amino-2-(4- chloro-3- dimethylamino-2- fluorophenyl)-5- ethenyl-6-(prop-2- enyloxy)carbonyl- pyrimidine | 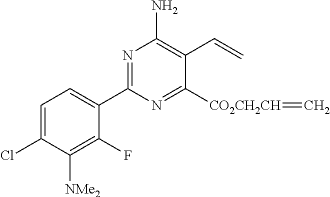 | 7.61 (1H, t), 7.22 (1H, dd), 6.78 (1H, dd), 6.02 (1H, m), 5.68 (1H, d), 5.65 (1H, dd), 5.47 (1H, m), 5.43 (2H, br s), 5.31 (1H, m), 4.85 (2H, m), 2.91 (6H, d) |
| 33-396 | 4-Amino-2-(4- chloro-3- dimethylamino-2- fluorophenyl)-5- ethenyl-6-(2- ethoxy- ethoxy)carbonyl- pyrimidine | 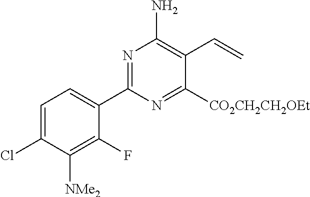 | 7.60 (1H, t), 7.22 (1H, dd), 6.79 (1H, dd), 5.69 (1H, d), 5.65 (1H, m), 5.41 (2H, br s), 4.50 (2H, m), 3.76 (2H, m), 3.58 (2H, q), 2.90 (6H, d), 1.25 (3H, t) |
| 33-397 | 4-Amino-6-(2- ⁿbutoxy- ethoxy)carbonyl-2- (4-chloro-3- dimethylamino-2- fluorophenyl)-5- ethenyl-pyrimidine | 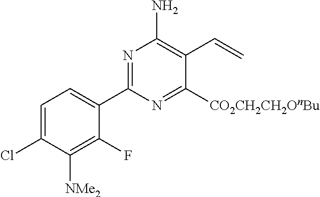 | 7.52 (1H, t), 7.24 (1H, dd), 6.77 (1H, dd), 5.75 (2H, m), 4.50 (2H, m), 3.75 (2H, m), 3.51 (2H, t), 3.29 (2H, br s), 2.91 (6H, d), 1.57 (2H, m), 1.38 (2H, m), 0.92 (3H, t) |

### Example 14

### Synthesis of 4-amino-2-cyclopropyl-6-methoxycarbonyl-5-(trimethylsilylethynyl)-pyrimidine (compound 2-78)

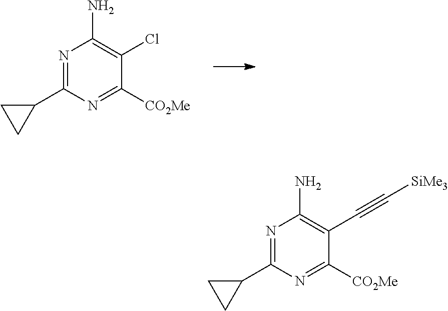

Palladium tetrakis(triphenylphosphine) (231 mg, 0.20 mmol) was added to a degassed solution of 4-amino-5-chloro-2-cyclopropyl-6-methoxycarbonylpyrimidine (prepared as described in example 2) (227 mg, 1.0 mmol) and trimethylsilylacetylene (0.28 ml, 2.0 mmol) in diisopropylamine (8 ml) and fluorobenzene (20 ml). The resulting solution was heated under nitrogen at 80° C. for 32 hours, then allowed to cool and the solvent evaporated under reduced pressure. The residue was dissolved in dichloromethane, washed with water and brine, dried over magnesium sulphate, filtered and evaporated under reduced pressure. The crude material was purified by high pressure liquid chromatography to provide 4-amino-2-cyclopropyl-6-methoxycarbonyl-5-(trimethylsilylethynyl)-pyrimidine as a white solid (15 mg, 5%).

¹H nmr (400 MHz, CDCl₃) δ_{H} 5.50 (2H, br s), 3.95 (3H, s), 2.10 (1H, m), 1.10 (2H, m), 1.00 (2H, m), 0.30 (9H, s) ppm.

### Example 15

### Synthesis of 4-amino-2-chloro-6-methoxycarbonyl-5-prop-1-ynyl-pyrimidine (compound 1-74)

### 15.1 Preparation of 2,4-dichloro-5-iodo-6-methoxycarbonylpyrimidine

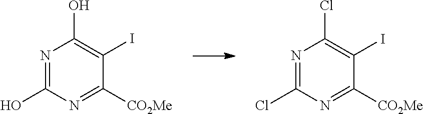

A mixture of 2,4-dihydroxy-5-iodo-6-methoxycarbonylpyrimidine (1.0 g, 3.38 mmol), phosphorus oxychloride (6 ml) and dimethylformamide (0.1 ml) was heated at reflux for 3 hours, then allowed to cool to room temperature, concentrated under reduced pressure and poured into iced water. The resulting mixture was extracted with dichloromethane and the combined organic extracts washed with brine, dried over magnesium sulphate, filtered and evaporated under reduced pressure. The residue was purified by chromatography on silica using a gradient of hexane/ethyl acetate (8:2 to 6:4) as eluent to provide 2,4-dichloro-5-iodo-6-methoxycarbonylpyrimidine as a brown solid (520 mg, 46%).

¹H nmr (400 MHz, CDCl₃) δ_{H} 4.00 (3H, s) ppm.

### 15.2 Preparation of 4-amino-2-chloro-5-iodo-6-methoxycarbonylpyrimidine

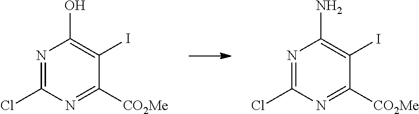

A solution of 2,4-dichloro-5-iodo-6-methoxycarbonylpyrimidine (950 mg, 2.86 mmol) and ammonia (7M in methanol; 1.6 ml, 11 mmol) in anhydrous dioxane (10 ml) was stirred at ambient temperature for 30 minutes then evaporated under reduced pressure. The residue was dissolved in ethyl acetate, washed with water, dried over magnesium sulphate, filtered and evaporated under reduced pressure. The residue was purified by chromatography on silica using a gradient of hexane/ethyl acetate (9:1 to 6:4) as eluent to provide 4-amino-2-chloro-5-iodo-6-methoxycarbonylpyrimidine as a white solid (590 mg, 66%).

¹H nmr (400 MHz, CDCl₃) δ_{H} 5.90 (2H, br s), 4.00 (3H, s) ppm.

### 15.3 Preparation of 4-amino-2-chloro-6-methoxycarbonyl-5-prop-1-ynyl-pyrimidine (compound 1-74)

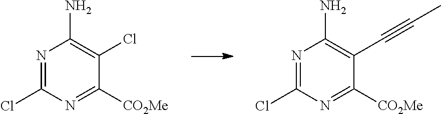

A solution of 4-amino-2-chloro-5-iodo-6-methoxycarbonylpyrimidine (210 mg, 0.67 mmol), 1-tributylstannyl-propyne (0.3 ml, 1 mmol) and bis(triphenylphosphine)palladium dichloride (48 mg, 0.067 mmol) in dimethylformamide (6 ml) was heated in a microwave reactor at 160° C. for 20 minutes, then allowed to cool. Ethyl acetate was added and the resulting solution washed with water and brine, dried over magnesium sulphate, filtered and evaporated. The residue was purified by chromatography using a gradient of hexane/ethyl acetate (9:1 to 2:8) as eluent to provide 4-amino-2-chloro-6-methoxycarbonyl-5-prop-1-ynyl-pyrimidine as a white solid (90 mg, 63%).

¹H nmr (400 MHz, CDCl₃) δ_{H} 4.00 (3H, s), 2.20 (3H, s) ppm (amine protons not observed).

### Example 16

### Synthesis of 4-amino-2-(4-chloro-2-fluoro-3-methoxyphenyl)-6-methoxycarbonyl-5-prop-1-ynyl-pyrimidine (compound 27-74)

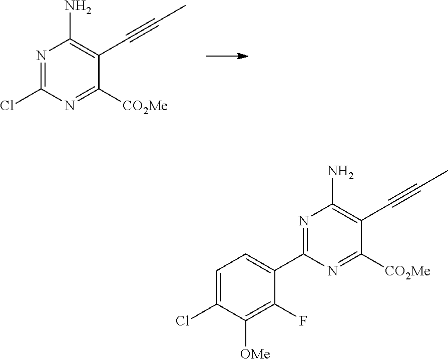

4-Chloro-2-fluoro-3-methoxyphenyl boronic acid (232 mg, 1.13 mmol), 4-amino-2-chloro-6-methoxycarbonyl-5-prop-1-ynyl-pyrimidine (prepared as described in example 15) (170 mg, 0.76 mmol), caesium fluoride (228 mg, 1.5 mmol) and [1,1′-bis(diphenylphosphino)-ferrocene] dichloropalladium (II) complex with dichloromethane (1:1) (62 mg, 0.075 mmol) were placed in a vial. The vial was evacuated and backfilled with nitrogen before adding dimethoxyethane (5 ml) and water (5 ml). The reaction mixture was heated in a microwave reactor at 140° C. for 20 minutes, then allowed to cool. Dichloromethane was added and the solution washed with water, dried over magnesium sulphate, filtered and evaporated under reduced pressure. The crude product was purified by chromatography on silica using a gradient of hexane/ethyl acetate (9:1 to 3:2) as eluent to provide 4-amino-2-(4-chloro-2-fluoro-3-methoxyphenyl)-6-methoxycarbonyl-5-prop-1-ynyl-pyrimidine as a white solid (30 mg, 11%).

¹H nmr (400 MHz, CDCl₃) δ_{H} 7.70 (1H, t), 7.20 (1H, dd), 5.80 (2H, br s), 4.00 (6H, s), 2.20 (3H, s) ppm.

### Example 17

### Synthesis of 4-amino-2-(4-chloro-2-fluoro-3-methoxyphenyl)-6-formyl-6-methoxycarbonyl-pyrimidine (compound 27-52)

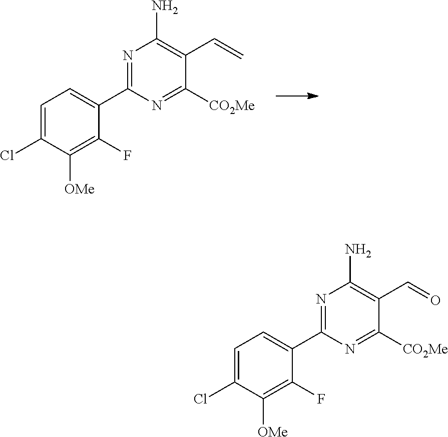

Ozone was bubbled through a stirred solution of 4-amino-2-(4-chloro-2-fluoro-3-methoxyphenyl)-5-ethenyl-6-methoxycarbonyl-pyrimidine (prepared by the method described in example 8) (125 mg, 0.0.37 mmol) in dichloromethane (40 ml) at −78° C. until a blue colour persisted. Oxygen was then bubbled through the solution until it became colourless. Dimethyl sulphide (2 ml) was added and the solution allowed to warm to ambient temperature over 2 hours. The reaction mixture was evaporated under reduced pressure and the residue purified by chromatography on silica using a gradient of hexane/ethyl acetate (100:0 to 60:40) to provide 4-amino-2-(4-chloro-2-fluoro-3-methoxyphenyl)-5-formyl-6-methoxycarbonyl-pyrimidine as an off-white solid (50 mg, 40%).

¹H nmr (400 MHz, CDCl₃) δ_{H} 10.40 (1H, s), 7.80 (1H, t), 7.20 (1H, m), 4.10 (3H, s), 4.00 (3H, s) ppm (amine protons not observed).

### Example 18

### Synthesis of 4-amino-5-(1-ethoxyethenyl)-6-methoxycarbonyl-2-(4-trifluoromethylphenyl)-pyrimidine (compound 31-67)

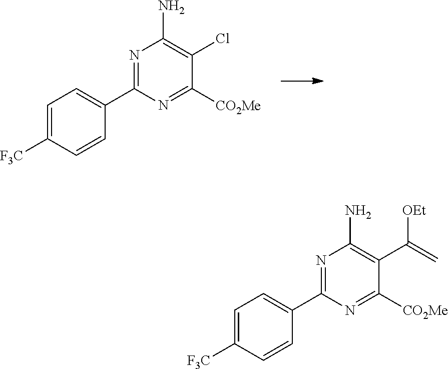

A solution of 4-amino-5-chloro-6-methoxycarbonyl-2-(4-trifluoromethylphenyl)-pyrimidine (prepared as described in example 5) (662 mg, 2.0 mmol), 1-ethoxy-1-tributylstannyl-ethene (0.8 ml, 2.4 mmol) and palladium tetrakis(triphenylphosphine) (462 mg, 0.40 mmol) in dimethylsulphoxide (14 ml) was heated at 170° C. in a microwave reactor for 30 minutes. The reaction mixture was allowed to cool, potassium fluoride (saturated solution in methanol; 30 ml) added and the resulting mixture stirred for 17 hours at ambient temperature. The mixture was filtered through Celite®, the solid washed with methanol and the filtrate concentrated under reduced pressure. The residue was extracted with ether and ethyl acetate and the combined organic extracts washed with brine, dried over magnesium sulphate, filtered and evaporated under reduced pressure. The crude material was purified column chromatography on silica using hexane/ethyl acetate (4:1) as eluent to provide 4-amino-5-(1-ethoxyethenyl)-6-methoxycarbonyl-2-(4-trifluoromethylphenyl)-pyrimidine as a pale yellow solid (651 mg, 89%). ¹H nmr (400 MHz, CDCl₃) δ_{H} 8.50 (2H, d), 7.80 (2H, d), 5.60 (2H, br s), 4.50 (2H, dd), 3.90 (2H, q), 3.90 (3H, s), 1.40 (3H, t) ppm.

Further examples, prepared using this general method, are listed in Table 11 below.

TABLE 11
Compounds made according to the general method described in
Example 18 above. Characteristic data is ¹H nmr data (400 MHz,
CDCl₃) δ_{H} ppm

| Compound | | | Characteristic |
|---|---|---|---|
| No. | Name | Structure | data |
| 33-67 | 4-Amino-2-(4-chloro- 3-dimethylamino-2- fluorophenyl)-5-(1- ethoxyethenyl)-6- methoxycarbonyl- pyrimidine | 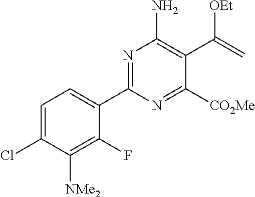 | 7.60 (1H, t), 7.20 (1H, dd), 5.60 (2H, br s), 4.50 (2H, dd), 3.90 (3H, s), 3.90 (2H, q), 2.90 (6H, s), 1.40 (3H, t) |
| 27-67 | 4-Amino-2-(4-chloro- 2-fluoro-3- methoxyphenyl)-5- (1-ethoxyethenyl)-6- methoxycarbonyl- pyrimidine | 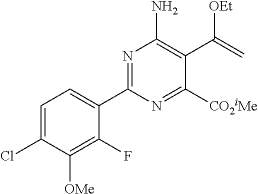 | 7.60 (1H, t), 7.20 (1H, dd), 5.60 (2H, br s), 4.50 (2H, dd), 4.00 (3H, d), 3.90 (3H, s), 3.90 (2H, q), 1.40 (3H, t) |

Also isolated from a reaction of this type was 4-amino-2-(4-[1-ethoxyethenyl]-2-fluoro-3-methoxy-phenyl)-6-methoxycarbonyl-5-methylcarbonyl-pyrimidine (compound 66-53).

¹H nmr (400 MHz, CDCl₃) δ_{H} 7.60 (1H, t), 7.50 (1H, dd), 5.70 (2H, br s), 4.50 (2H, dd), 4.00 (3H, d), 3.90 (3H, s), 3.90 (2H, q), 2.60 (3H, s), 1.40 (3H, t) ppm.

### Example 19

### Synthesis of 4-amino-6-methoxycarbonyl-5-methylcarbonyl-2-(4-trifluoromethylphenyl)-pyrimidine (compound 31-53)

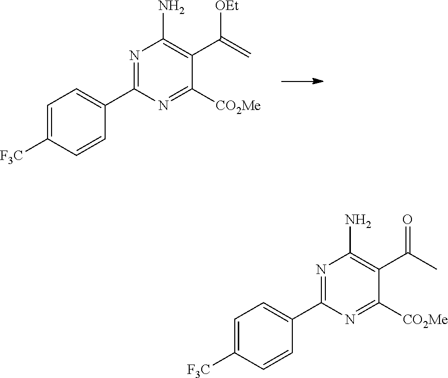

A solution of 4-amino-5-(1-ethoxyethenyl)-6-methoxycarbonyl-2-(4-trifluoromethylphenyl)-pyrimidine (prepared as described in Example 18) (300 mg, 0.82 mmol) and hydrochloric acid (1M; 6 ml, 6 mmol) in tetrahydrofuran (12 ml) was stirred at ambient temperature for 1 hour. The reaction mixture was concentrated under reduced pressure, extracted with ethyl acetate and the combined organic extracts washed with aqueous sodium hydrogen carbonate and brine, dried over magnesium sulphate, filtered and evaporated under reduced pressure. The crude product was purified by column chromatography on silica using a gradient of hexane/ethyl acetate (100:0 to 4:1) as eluent to provide 4-amino-6-methoxycarbonyl-5-methylcarbonyl-2-(4-trifluoromethylphenyl)-pyrimidine as an off white solid (241 mg, 87%).

¹H nmr (400 MHz, CDCl₃) δ_{H} 8.50 (2H, d), 7.70 (2H, d), 4.00 (3H, s), 3.48 (2H, br s), 2.50 (3H, s) ppm.

Further examples, prepared using this general method, are listed in Table 12 below.

TABLE 12
Compounds made according to the general method described in
Example 19 above. Characteristic data is ¹H nmr data (400 MHz,
CDCl₃) δ_{H} ppm

| Compound | | | Characteristic |
|---|---|---|---|
| No. | Name | Structure | data |
| 33-53 | 4-Amino-2-(4-chloro- 3-dimethylamino-2- fluorophenyl)-6- methoxycarbonyl-5- methylcarbonyl- pyrimidine | 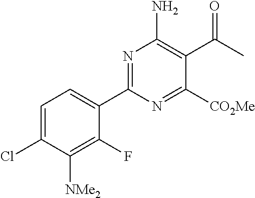 | 7.70 (1H, t), 7.20 (1H, dd), 4.00 (3H, s), 2.90 (6H, s), 2.50 (3H, s) (amine protons not observed) |
| 27-53 | 4-Amino-2-(4-chloro- 2-fluoro-3-methoxy- phenyl)-6- methoxycarbonyl-5- methylcarbonyl- pyrimidine | 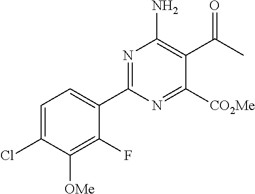 | 7.70 (1H, t), 7.20 (1H, dd), 4.10 (3H, s), 4.00 (3H, s), 2.50 (3H, s) (amine protons not observed) |
| 65-53 | 4-Amino-2-(2-fluoro- 3-methoxy-4- methylcarbonyl- phenyl)-6- methoxycarbonyl-5- methylcarbonyl- pyrimidine | 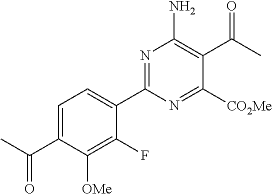 | 7.70 (1H, t), 7.50 (1H, dd), 4.10 (3H, s), 4.00 (3H, s), 2.70 (3H, s), 2.50 (3H, s) (amine protons not observed) |

### Example 20

### Synthesis of 4-amino-5-(1-hydroxyethyl)-6-hydroxymethyl-2-(4-trifluoromethylphenyl)-pyrimidine (compound 31-372)

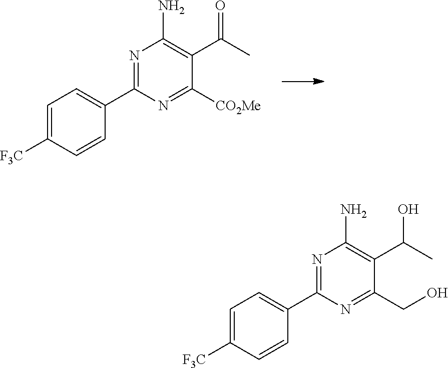

Sodium borohydride (11 mg, 0.30 mmol) was added to a stirred solution of 4-amino-6-methoxycarbonyl-5-methylcarbonyl-2-(4-trifluoromethylphenyl)-pyrimidine (prepared as described in example 19) (51 mg, 0.15 mmol) in methanol (2 ml). The reaction mixture was stirred at ambient temperature for 1 hour, then 1N hydrochloric acid (1 ml) was added and the mixture concentrated under reduced pressure and extracted with ethyl acetate. The combined organic extracts were washed with brine, dried over magnesium sulphate, filtered and evaporated under reduced pressure. The residue was purified using a FractionLynx automated hplc system to provide 4-amino-5-(1-hydroxyethyl)-6-hydroxymethyl-2-(4-trifluoromethylphenyl)-pyrimidine as a colourless oil (13 mg, 28%).

¹H nmr (400 MHz, CDCl₃) δ_{H} 8.40 (2H, d), 7.70 (2H, d), 6.00 (2H, br s), 5.00 (1H, q), 4.80 (1H, br s), 4.60 (2H, q), 2.70 (1H, br s), 1.50 (3H, d) ppm.

### Example 21

### Synthesis of 4-amino-5-ethyl-6-methoxycarbonyl-2-(4-trifluoro-methylphenyl)-pyrimidine (compound 31-42)

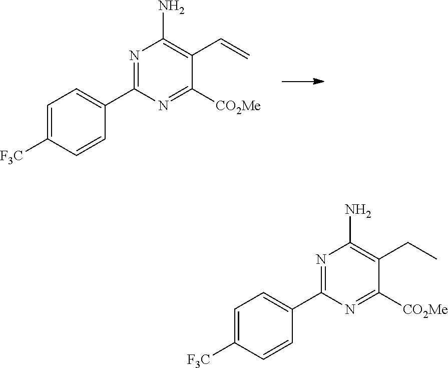

A solution of 4-amino-5-ethenyl-6-methoxycarbonyl-2-(4-trifluoromethylphenyl)-pyrimidine (prepared using the method described in example 8) (38 mg, 0.11 mmol) in methanol (10 ml) was reduced using a Thales H-cube reactor fitted with a 30 mm palladium on carbon cartridge. The reaction was performed at 30° C. and 20 bar pressure at a flow rate of 1 ml/minute. The reaction solution was evaporated under reduced pressure to provide 4-amino-5-ethyl-6-methoxycarbonyl-2-(4-trifluoromethylphenyl)-pyrimidine (38 mg, 99%).

M.p. 123-124° C.; ¹H nmr (400 MHz, CDCl₃) δ_{H} 8.50 (2H, d), 7.70 (2H, d), 5.30 (2H, br s), 4.00 (3H, s), 2.70 (2H, q), 1.30 (3H, t) ppm.

Further compounds, prepared using this general method, are listed in Table 13 below.

TABLE 13
Compounds made according to the general method described in Example
21 above. Characteristic data provided is ¹H-nmr data (400 MHz, CDCl₃) δ_{H}
ppm

| Compound | | | |
|---|---|---|---|
| No. | Name | Structure | Characteristic data |
| 31-43 | 4-Amino-6- methoxycarbonyl- 5-propyl-2-(4- trifluoromethyl- phenyl)-pyrimidine | 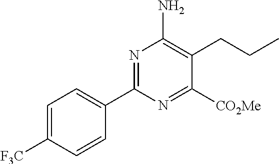 | 8.50 (2H, d), 7.70 (2H, d), 5.20 (2H, br s), 4.00 (3H, s), 2.60 (2H, q), 1.70 (2H, septet), 1.10 (3H, t) |

### Example 22

### Synthesis of 4-amino-2-(3-dimethylamino-4-ethenyl-2-fluorophenyl)-5-ethenyl-6-methoxycarbonylpyrimidine (compound 64-61)

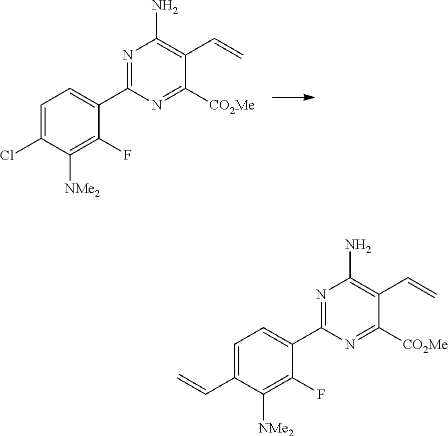

A mixture of 4-amino-2-(4-chloro-3-dimethylamino-2-fluorophenyl)-5-ethenyl-6-methoxycarbonylpyrimidine (prepared as described in example 8) (200 mg, 0.57 mmol), vinyl boronic acid pinacol ester (0.12 ml, 0.69 mmol), palladium acetate (6 mg, 0.029 mmol), tricyclohexylphosphine tetrafluoroborate (21 mg, 0.057 mmol), potassium phosphate (363 mg, 1.7 mmol), toluene (3.6 ml) and water (0.4 ml) was purged with nitrogen, then heated in a microwave reactor at 180° C. for 30 minutes. The reaction mixture was allowed to cool, water added and the resulting mixture extracted with dichloromethane. The organic phase was washed with brine, dried over magnesium sulphate, filtered and evaporated under reduced pressure. The residue was purified by chromatography on silica using hexane/ethyl acetate (first 8:2, then 6:4) as eluent to provide 4-amino-2-(3-dimethylamino-4-ethenyl-2-fluorophenyl)-5-ethenyl-6-methoxycarbonylpyrimidine as a yellow oil (95 mg, 49%).

¹H nmr (400 MHz, CDCl₃) δ_{H} 7.60 (1H, t), 7.35 (1H, d), 7.20 (1H, dd), 6.80 (1H, dd), 5.70 (3H, m), 5.40 (2H, br s), 5.30 (1H, d), 3.90 (3H, s), 2.80 (6H, s) ppm.

### Example 23

### Synthesis of 4-chloro-2-fluoro-3-methoxybenzamidine

### 23.1 Preparation of 3-bromo-6-chloro-2-fluorophenol

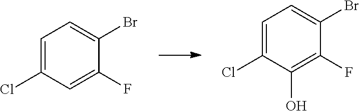

n-Butyllithium (2.1M in hexane; 68 ml, 149 mmol) was added dropwise to a stirred solution of diisopropylamine (15 g, 149 mmol) in anhydrous tetrahydrofuran (700 ml) at −78° C. under a nitrogen atmosphere. Once the addition was complete the reaction mixture was allowed to warm to 0° C., cooled to −78° C. and a solution of 1-bromo-4-chloro-2-fluorobenzene (25 g, 119 mmol) in anhydrous tetrahydrofuran (60 ml) was added dropwise. The mixture was allowed to warm to −20° C., cooled to −78° C. and a solution of trimethylborate (15 g, 143 mmol) in anhydrous tetrahydrofuran (30 ml) added dropwise. The reaction mixture was allowed to warm to −20° C. and stirred at that temperature for 30 minutes, then cooled to −78° C. and peracetic acid (80 ml) added dropwise. The mixture was allowed to warm to ambient temperature and stirred overnight under nitrogen. Water (11) was added and the resulting mixture extracted with ethyl acetate (3×500 ml). The combined organic extracts were washed with water and brine, dried over sodium sulphate, filtered and evaporated under reduced pressure. The residue was purified by column chromatography on silica using 2% ethyl acetate in hexane as eluent to provide 3-bromo-6-chloro-2-fluorophenol (13.8 g, 51%).

### 23.2 Preparation of 1-bromo-4-chloro-2-fluoro-3-methoxybenzene

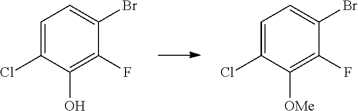

Anhydrous potassium carbonate (17 g, 122 mmol) was added to a solution of 3-bromo-6-chloro-2-fluorophenol (13.8 g, 61 mmol) in anhydrous acetonitrile (100 ml). at ambient temperature. Methyl iodide (17 g, 122 mmol) was then added dropwise. The resulting mixture was heated at reflux for 2 hours, then cooled to ambient temperature and filtered through Celite®, the solid being washed with acetonitrile. The filtrate was evaporated under reduced pressure, the residue dissolved in ethyl acetate (250 ml) and washed with water (100 ml). The aqueous phase was extracted with ethyl acetate (100 ml and the combined organic phases were washed with brine, dried over sodium sulphate, filtered and evaporated under reduced pressure. The crude product was purified by column chromatography on silica using hexane as eluent to provide 1-bromo-4-chloro-2-fluoro-3-methoxybenzene as a colourless oil, which solidified on storage at 5° C. (11.4 g, 78%).

### 23.3 Preparation of 4-chloro-2-fluoro-3-methoxybenzonitrile

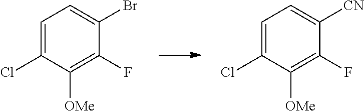

Copper(I) cyanide (57 g, 0.64 mol) was added to a degassed solution of 1-bromo-4-chloro-2-fluoro-3-methoxybenzene (76 g, 0.32 mol) in anhydrous dimethylformamide (760 ml). The resulting mixture was again degassed and tetrakis(triphenylphosphine) palladium(0) (1.2 g) added. The reaction mixture was heated at 110° C. under nitrogen for 24 hours, then allowed to cool to ambient temperature and water (2.5 l) added. The resulting mixture was stirred for 10 minutes, then filtered through Celite® and the solid washed with ethyl acetate (500 ml). The filtrate was separated into phases and the aqueous extracted with ethyl acetate (3×500 ml). The combined organic phases were washed with water and brine, dried over sodium sulphate, filtered and evaporated under reduced pressure. The crude product which was purified by column chromatography on silica using agradient of ethyl acetate (2-5%) in hexane as eluent to provide 4-chloro-2-fluoro-3-methoxybenzonitrile as a colourless solid (38 g, 65%).

### 23.4 Preparation of 4-chloro-2-fluoro-3-methoxybenzamidine

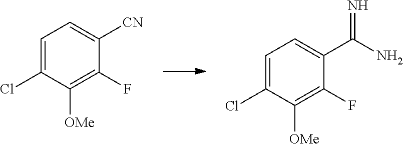

n-Butyllithium (2.2M in hexane; 196 ml, 0.43 mol) was added dropwise to a solution of hexamethyldisilazane (72 g, 0.45 mol) in anhydrous diethyl ether (800 ml) at −15° C. under a nitrogen atmosphere. The resulting solution was stirred at −15° C. for 1 hour, then a solution of 4-chloro-2-fluoro-3-methoxybenzonitrile (40 g, 0.22 mol) in anhydrous diethyl ether (600 ml) was added dropwise. The reaction mixture was stirred at −15° C. for 30 minutes, then allowed to warm to ambient temperature and stirring continued for 2 hours. The mixture was cooled to −10° C., hydrochloric acid (3M; 360 ml) added dropwise and the resulting mixture stirred at 0° C. for 45 minutes. The phases were separated and the aqueous layer washed with ethyl acetate (250 ml). The aqueous phase was cooled to 0° C., basified by the addition of aqueous sodium hydroxide (3M) and extracted with ethyl acetate (3×300 ml). The combined organic extracts were dried over sodium sulphate, filtered and evaporated under reduced to provide 4-chloro-2-fluoro-3-methoxybenzamidine as a yellow solid (33 g, 75%).

### Example 24

### Synthesis of 4-chloro-6-ethoxycarbonyl-5-methyl-2-(4-trifluoromethylphenyl)-pyrimidine (compound 31-390)

### 24.1 Preparation of 6-ethoxycarbonyl-4-hydroxy-5-methyl-2-(4-trifluoromethylphenyl)-pyrimidine and 4,6-dihydroxy-6-ethoxycarbonyl-5-methyl-2-(4-trifluoromethylphenyl)-3,4-dihydropyrimidine

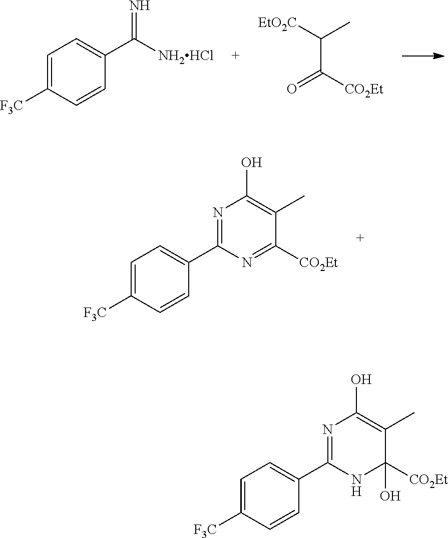

A suspension of sodium ethoxide (940 mg, 13.8 mmol) in ethanol (4.5 ml) was added to a solution of 4-(trifluoromethyl)benzamidine hydrochloride dihydrate (2.79 g, 12.4 mmol) and diethyl oxalpropiolate (2.6 ml, 13.8 mmol) in ethanol (30 ml) and the resulting mixture stirred at ambient temperature for 18 hours. The reaction mixture was concentrated under reduced pressure, then dichloromethane was added and the solution dried over magnesium sulphate, filtered and evaporated under reduced pressure. The crude product was purified by chromatography on silica using hexane/ethyl acetate (3:2) as eluent to provide a mixture of 6-ethoxycarbonyl-4-hydroxy-5-methyl-2-(4-trifluoromethylphenyl)-pyrimidine and 4,6-dihydroxy-6-ethoxycarbonyl-5-methyl-2-(4-trifluoromethylphenyl)-3,4-dihydropyrimidine as a pale brown solid (2.21 g).

### 24.2 Preparation of 4-chloro-6-ethoxycarbonyl-5-methyl-2-(4-trifluoromethylphenyl)-pyrimidine (compound 31-390)

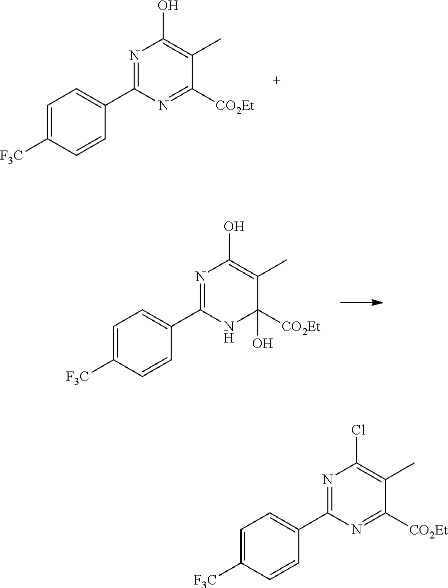

Phosphorus oxychloride (5 ml) was added to a mixture of 6-ethoxycarbonyl-4-hydroxy-5-methyl-2-(4-trifluoromethylphenyl)-pyrimidine and 4,6-dihydroxy-6-ethoxycarbonyl-5-methyl-2-(4-trifluoromethylphenyl)-3,4-dihydropyrimidine (640 mg, 1.9 mmol) in a sealed vial. The resulting mixture was heated at 105° C. for 3 hours, then cooled to ambient temperature and allowed to stand for 18 hours. The mixture was poured onto ice and solid sodium hydrogen carbonate added to bring the pH to 7. The resulting mixture was extracted with ethyl acetate and the combined organic extracts washed with brine, dried over magnesium sulphate, filtered and evaporated under reduced pressure to provide 4-chloro-6-ethoxycarbonyl-5-methyl-2-(4-trifluoromethylphenyl)-pyrimidine as a brown oil (142 mg).

MH⁺ 345, 347.

### Example 25

### Synthesis of 4-dimethylamino-6-ethoxycarbonyl-5-methyl-2-(4-trifluoromethylphenyl)-pyrimidine (compound 31-301)

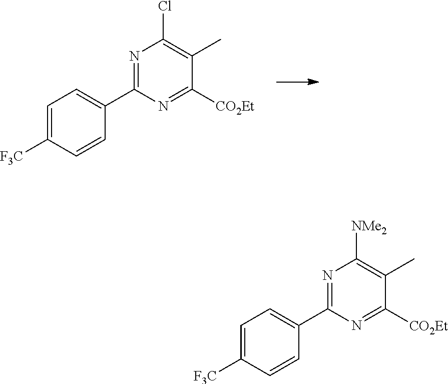

A solution of 4-chloro-6-ethoxycarbonyl-5-methyl-2-(4-trifluoromethylphenyl)-pyrimidine (prepared as described in example 24) (100 mg, 0.29 mmol), dimethylamine hydrochloride (47 mg, 0.58 mmol) and triethylamine (0.08 ml, 0.58 mmol) in dichloromethane (2 ml) was stirred at ambient temperature for 2.5 hours. Ethyl acetate was added and the resulting solution washed with brine, dried over magnesium sulphate, filtered and evaporated under reduced pressure. A sub-sample of the residue was purified using a FractionLynx automated hplc system to provide 4-dimethylamino-6-ethoxycarbonyl-5-methyl-2-(4-trifluoromethylphenyl)-pyrimidine as a yellow oil (7 mg)

¹H nmr (400 MHz, CDCl₃) δ_{H} 8.50 (2H, d), 7.70 (2H, d), 4.50 (2H, q), 3.20 (6H, s), 2.40 (3H, s), 1.50 (3H, t) ppm.

### Example 26

### Synthesis of 5-chloro-2-cyclopropyl-4-methoxy-6-methoxycarbonyl-pyrimidine

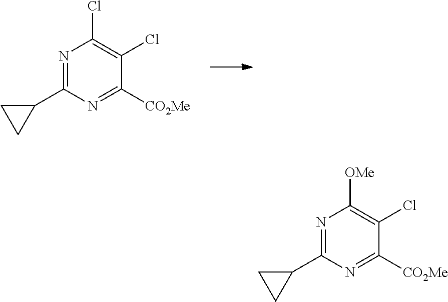

Triethylamine (1 ml, 2.5 mmol) was added to a stirred solution of 2-cyclopropyl-4,5-dichloro-6-methoxycarbonylpyrimidine (prepared as described in example 1) (500 mg, 2.0 mmol) in methanol (10 ml). The mixture was heated at 45° C. for 14 hours, allowed to stand at ambient temperature for 72 hours, and then evaporated under reduced pressure. The residue was dissolved in ethyl acetate and the resulting solution washed with water and brine, dried over magnesium sulphate, filtered and evaporated under reduced pressure to provide 5-chloro-2-cyclopropyl-4-methoxy-6-methoxycarbonyl-pyrimidine as a white solid (400 mg, 81%).

¹H nmr (400 MHz, CDCl₃) δ_{H} 4.03 (3H, s), 4.00 (3H, s), 2.20 (1H, m), 1.12 (2H, m), 1.08 (2H, m) ppm.

Further examples, prepared using this general method, are listed in Table 14 below.

TABLE 14
Compounds made according to the general method described in
Example 26 above.

| | | Melting |
|---|---|---|
| | | point |
| Name | Structure | (° C.) |
| 5-Chloro-2-(4- chloro-2-fluoro- 3-methoxy- phenyl)-4- methoxy-6- methoxy- carbonyl- pyrimidine | 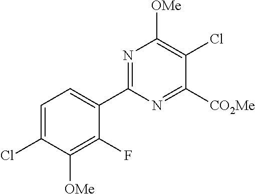 | 143-145 |

### Example 27

### Synthesis of 2-(4-chloro-2-fluoro-3-methoxyphenyl)-5-ethenyl-4-methoxy-6-methoxycarbonyl-pyrimidine (compound 27-398)

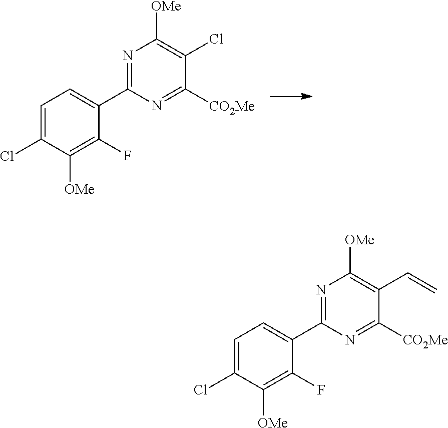

A mixture of vinyl boronic acid pinacol ester (0.1 ml, 0.58 mmol), 5-chloro-2-(4-chloro-2-fluoro-3-methoxyphenyl)-4-methoxy-6-methoxycarbonylpyrimidine (prepared as described in example 26) (200 mg, 0.55 mmol), caesium fluoride (168 mg, 1.1 mmol), [1,1′-bis(diphenylphosphino)-ferrocene] dichloropalladium (II) complex with dichloromethane (1:1) (46 mg, 0.056 mmol), dimethoxyethane (2 ml) and water (2 ml) was heated in a microwave reactor at 150° C. for 20 minutes, then allowed to cool. Ethyl acetate was added and the solution washed with brine, dried over magnesium sulphate, filtered and evaporated under reduced pressure to leave a brown gum, which was purified by chromatography on silica using hexane/ethyl acetate (9:1 then 4:1) as eluent to provide 2-(4-chloro-2-fluoro-3-methoxyphenyl)-5-ethenyl-4-methoxy-6-methoxycarbonyl-pyrimidine as a white solid (115 mg, 59%).

M.p. 107-108° C.; ¹H nmr (400 MHz, CDCl₃) δ_{H} 7.82 (1H, t), 7.24 (1H, d), 6.82 (1H, q), 6.06 (1H, d), 5.63 (1H, d), 4.17 (3H, s), 4.04 (6H, s), 3.97 (3H, s) ppm.

Further examples, prepared using this general method, are listed in Table 15 below.

TABLE 15
Compounds made according to the general method described in
Example27 above. Characteristic data is ¹H nmr data (400 MHz,
CDCl₃) δ_{H} ppm

| Com- | | | |
|---|---|---|---|
| pound | | | Characteristic |
| No. | Name | Structure | data |
| 2-398 | 2-Cyclopropyl- 5-ethenyl-4- methoxy-6- methoxy- carbonyl- pyrimidine | 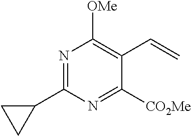 | 6.71 (1H, dd), 5.89 (1H, d), 5.50 (1H, d), 4.00 (3H, s), 3.95 (3H, s), 2.20 (1H, m), 1.14 (2H, m), 1.03 (2H, m) |

### Example 28

### Synthesis of 2-cyclopropyl-5-ethenyl-6-methoxycarbonyl-4-methylthio-pyrimidine (compound 2-400)

### 28.1 Preparation of 5-chloro-2-cyclopropyl-6-methoxycarbonyl-4-methylthio-pyrimidine

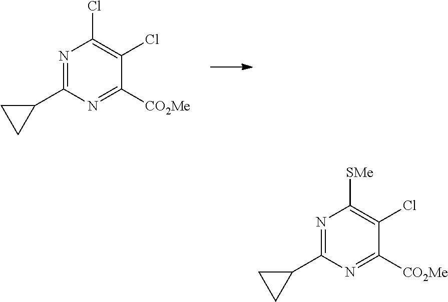

Sodium methanethiolate (1.0 g, 14 mmol) was added portionwise to a stirred solution of 2-cyclopropyl-4,5-dichloro-6-methoxycarbonylpyrimidine (prepared as described in example 1) (2.46 g, 10 mmol) in methanol (50 ml). The resulting mixture was stirred at ambient temperature for 1.5 hours, then evaporated under reduced pressure. The residue was partitioned between ethyl acetate and water and the organic phase washed with water and brine, dried over magnesium sulphate, filtered and evaporated to provide 5-chloro-2-cyclopropyl-6-methoxycarbonyl-4-methylthio-pyrimidine as a white solid (2.1 g, 80%).

M.p. 74-75° C.; ¹H nmr (400 MHz, CDCl₃) δ_{H} 4.00 (3H, s), 2.50 (3H, s), 2.24 (1H, m), 1.17 (2H, m), 1.10 (2H, m) ppm.

### 28.2 Preparation of 2-cyclopropyl-5-ethenyl-6-methoxycarbonyl-4-methylthio-pyrimidine (compound 2-400)

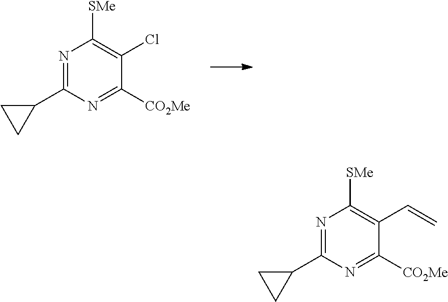

A mixture of vinyl boronic acid pinacol ester (0.4 ml, 2 mmol), 5-chloro-2-cyclopropyl-6-methoxycarbonyl-4-methylthio-pyrimidine (258 mg, 1.0 mmol), caesium fluoride (0.6 g, 4 mmol), [1,1′-bis(diphenylphosphino)-ferrocene] dichloropalladium (II) complex with dichloromethane (1:1) (200 mg, 0.25 mmol), dimethoxyethane (3 ml) and water (2 ml) was heated in a microwave reactor at 140° C. for 1 hour, then allowed to cool. Ethyl acetate was added and the solution washed with water and brine, dried over magnesium sulphate, filtered and evaporated under reduced pressure to leave a brown oil, which was purified by chromatography on silica using hexane/ethyl acetate (4:1) as eluent to provide 2-cyclopropyl-5-ethenyl-6-methoxycarbonyl-4-methylthio-pyrimidine as a colourless gum (150 mg, 60%).

¹H nmr (400 MHz, CDCl₃) δ_{H} 6.68 (1H, m), 5.55 (2H, m), 3.90 (3H, s), 2.50 (3H, s), 2.23 (1H, m), 1.20 (2H, m), 1.08 (2H, m) ppm.

### Example 29

### Synthesis of (i) 2-cyclopropyl-5-ethenyl-6-methoxycarbonyl-4-methylsulphinyl-pyrimidine (compound 2-401) and (ii) 2-cyclopropyl-5-ethenyl-6-methoxycarbonyl-4-methylsulphonyl-pyrimidine (compound 2-402)

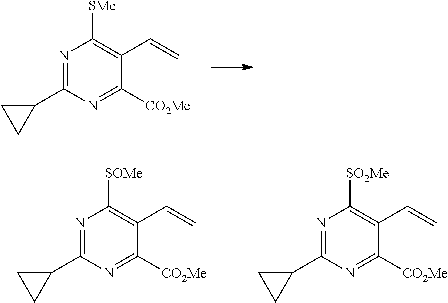

Peracetic acid (32% solution in dilute acetic acid; 0.25 ml, 1.1 mmol) was added dropwise to a stirred solution of 2-cyclopropyl-5-ethenyl-6-methoxycarbonyl-4-methylthio-pyrimidine (prepared as described in example 28) (125 mg, 0.5 mmol) in dichloromethane (5 ml) at 0° C. The reaction mixture was stirred at ambient temperature for 1 hour, then further dichloromethane added and the resulting solution washed with water and brine, dried over magnesium sulphate, filtered and evaporated under reduced pressure to leave a yellow gum. This was purified by chromatography on silica using hexane/ethyl acetate (4:1) as eluent to provide 2-cyclopropyl-5-ethenyl-6-methoxycarbonyl-4-methylsulphinyl-pyrimidine as a white solid (40 mg, 30%).

M.p. 74-75° C.; ¹H nmr (400 MHz, CDCl₃) δ_{H} 7.02 (1H, dd), 5.67 (1H, d), 5.50 (1H, d), 3.95 (3H, s), 2.88 (3H, s), 2.42 (1H, m), 1.27 (2H, m), 1.10 (2H, m) ppm.

Also isolated, as a yellow solid, was 2-cyclopropyl-5-ethenyl-6-methoxycarbonyl-4-methylsulphonyl-pyrimidine (10 mg, 7%).

¹H nmr (400 MHz, CDCl₃) δ_{H} 7.18 (1H, dd), 5.65 (1H, d), 5.58 (1H, d), 3.94 (3H, s), 3.33 (3H, s), 2.38 (1H, m), 1.20 (4H, m) ppm.

### Example 30

### Synthesis of 4-azido-2-cyclopropyl-5-ethenyl-6-methoxycarbonyl-pyrimidine (compound 2-399)

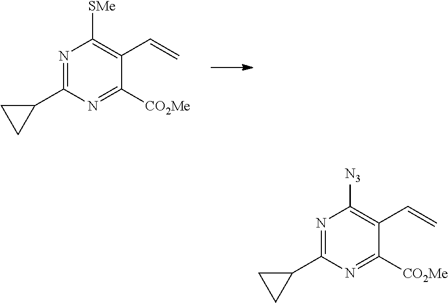

m-Chloroperbenzoic acid (516 mg, 3.0 mmol) was added portionwise to a stirred solution of 2-cyclopropyl-5-ethenyl-6-methoxycarbonyl-4-methylthio-pyrimidine (prepared as described in example 28) (250 mg, 1.0 mmol) in dichloromethane (10 ml) at 0° C. The reaction mixture was allowed to stand at ambient temperature for 16 hours, then further dichloromethane added and the resulting solution washed with water and brine, dried over magnesium sulphate, filtered and evaporated under reduced pressure to leave a gum. This was dissolved in methanol (10 ml) and sodium azide (500 mg, 7.7 mmol) added portionwise. The reaction mixture was then allowed to stand at ambient temperature for 64 hours, filtered and the filtrate evaporated under reduced pressure. The residue was purified by chromatography on silica using hexane/ethyl acetate (4:1) as eluent to provide 4-azido-2-cyclopropyl-5-ethenyl-6-methoxycarbonylpyrimidine as a white solid (65 mg, 27%).

M.p. 120-130° C. (decomp.); ¹H nmr (400 MHz, CDCl₃) δ_{H} 7.18 (1H, dd), 5.65 (1H, d), 5.58 (1H, d), 3.94 (3H, s), 2.38 (1H, m), 1.60 (2H, m), 1.46 (2H, m) ppm.

### Example 31

### Synthesis of (i) 2-cyclopropyl-4-hydroxy-6-methoxycarbonyl-methoxy-5-methylpyrimidine and (ii) 4,6-bis(methoxycarbonyl-methoxy)-2-cyclopropyl-5-methylpyrimidine (compound 2-384)

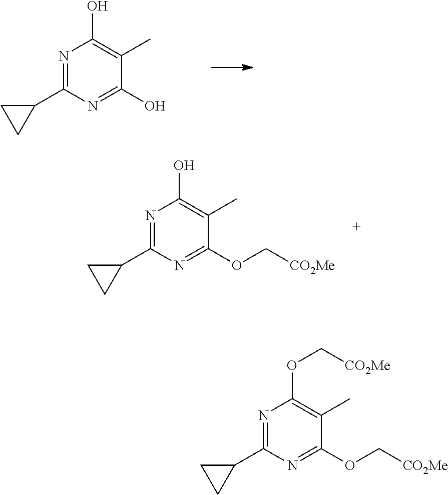

A mixture of 2-cyclopropyl-4,6-dihydroxy-5-methylpyrimidine (1.6 g, 9.6 mmol), methyl bromoacetate (1.5 g, 9.8 mmol), potassium carbonate (1.4 g, 10 mmol) and dimethylformamide (7.5 ml) was heated at 90° C. for 6 hours, then allowed to cool and stood at ambient temperature for 65 hours. Water was added and the resulting precipitate washed with ether and dried to provide a white solid. This was washed with ethyl acetate to provide 2-cyclopropyl-4-hydroxy-6-methoxycarbonylmethoxy-5-methylpyrimidine as a white solid (450 mg, 19%).

¹H nmr (400 MHz, CDCl₃) δ_{H} 13.80 (1H, br s), 4.70 (2H, s), 3.70 (3H, s), 1.90 (4H, m), 1.05 (4H, m) ppm.

The combined ethyl acetate washings were evaporated under reduced pressure and the residue purified by column chromatography on silica using ethyl acetate as eluent to provide 4,6-bis(methoxycarbonylmethoxy) 2-cyclopropyl-5-methylpyrimidine as a solid (450 mg, 15%).

¹H nmr (400 MHz, CDCl₃) δ_{H} 4.80 (4H, br s), 3.75 (6H, s), 2.10 (3H, s), 1.95 (1H, m), 0.90 (4H, m) ppm.

Further compounds, prepared using this general method, are listed in Table 16 below.

TABLE 16
Compounds made according to the general method described in Example
31 above. Characteristic data provided is ¹H-nmr data (400 MHz, CDCl₃)
δ_{H} ppm

| Com- | | | Character- |
|---|---|---|---|
| pound | | | istic |
| No. | Name | Structure | data |
| 2-389 | 4,6-bis(t- butoxy- carbonyl- methoxy)- 2-cyclo- propyl- 5-methyl- pyrimidine | 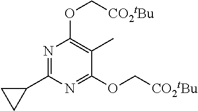 | 4.85 (2H, s), 4.60 (2H, s), 1.90 (3H, s), 1.65 (1H, m), 1.45 (18H, s), 1.10 (2H, m), 0.90 (2H, m) |

### Example 32

### Synthesis of 4-chloro-2-cyclopropyl-6-methoxycarbonylmethoxy-5-methylpyrimidine (compound 2-376)

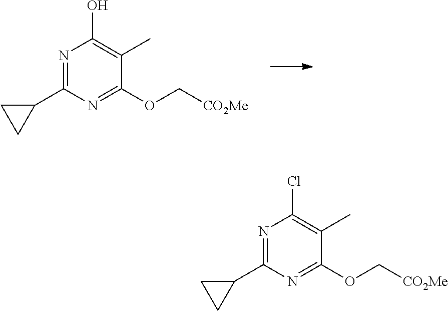

A stirred suspension of 2-cyclopropyl-4-hydroxy-6-methoxycarbonylmethoxy-5-methylpyrimidine (prepared as described in example 31) (450 mg, 1.9 mmol) in phosphorus oxychloride (3 ml) was heated at 120° C. for 5 hours. The reaction mixture was allowed to cool and evaporated under reduced pressure. The residue was dissolved in dichloromethane and the solution washed with saturated aqueous sodium bicarbonate, dried over magnesium sulphate, filtered and evaporated under reduced pressure. The residue was purified by column chromatography on silica using diethyl ether as eluent to provide 4-chloro-2-cyclopropyl-6-methoxycarbonylmethoxy-5-methylpyrimidine as a solid (350 mg, 70%).

¹H nmr (400 MHz, CDCl₃) δ_{H} 4.85 (2H, s), 3.75 (3H, s), 2.20 (3H, s), 2.10 (1H, m), 1.00 (4H, m) ppm.

### Example 33

### Synthesis of 4-amino-2-cyclopropyl-6-methoxycarbonylmethoxy-5-methylpyrimidine (compound 2-362)

### 33.1 Preparation of 4-azido-2-cyclopropyl-6-methoxycarbonylmethoxy-5-methylpyrimidine (compound 2-388)

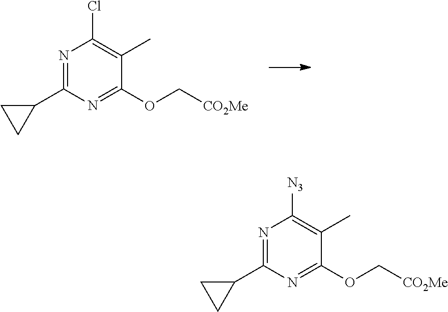

A stirred solution of 4-chloro-2-cyclopropyl-6-methoxycarbonylmethoxy-5-methylpyrimidine (prepared as described in example 32) (256 mg, 1.0 mmol) and sodium azide (100 mg, 1.5 mmol) in dimethylformamide (10 ml) was heated at 90° C. for 3 hours. The reaction mixture was allowed to cool and added to water. Ether was added, the mixture stirred for 30 minutes, then the phases separated. The organic phase was dried over magnesium sulphate, filtered and evaporated under reduced pressure to provide 4-azido-2-cyclopropyl-6-methoxycarbonylmethoxy-5-methylpyrimidine as an oil (200 mg, 76%).

MH⁺: 264.

### 33.2 Preparation of 4-amino-2-cyclopropyl-6-methoxycarbonylmethoxy-5-methylpyrimidine (compound 2-362)

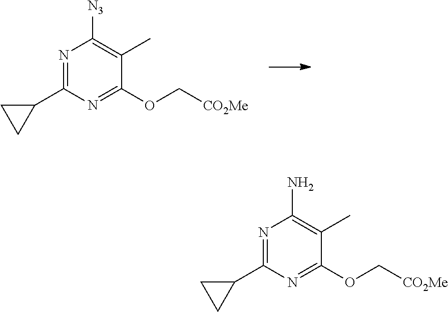

A mixture of 4-azido-2-cyclopropyl-6-methoxycarbonylmethoxy-5-methylpyrimidine (200 mg, 0.76 mmol) and palladium (5% on carbon; catalytic) in methanol (20 ml) was stirred under an atmosphere of hydrogen (4 bar) for 5 minutes. The reaction mixture was allowed to cool and added to water. Ether was added, the mixture stirred for 30 minutes, then the phases separated. The organic phase was dried over magnesium sulphate, filtered and evaporated under reduced pressure to provide 4-amino-2-cyclopropyl-6-methoxycarbonylmethoxy-5-methylpyrimidine.

¹H nmr (400 MHz, CDCl₃) δ_{H} 4.85 (2H, s), 3.75 (3H, s), 2.20 (3H, s), 2.10 (1H, m), 1.00 (4H, m) ppm (amino protons not observed).

### Example 34

### Synthesis of (4-chloro-2-cyclopropyl-5-methyl-pyrimidin-6-yloxy)-acetic acid (compound 2-375)

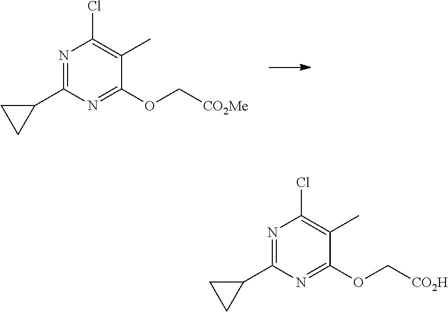

A solution of 4-chloro-2-cyclopropyl-6-methoxycarbonylmethoxy-5-methylpyrimidine (prepared as described in example 32) (256 mg, 1.0 mmol) and ammonia (7M in methanol; 0.6 ml, 4 mmol) in methanol (2.5 ml) was heated in a microwave reactor at 120° C. for 20 minutes, then cooled and evaporated under reduced pressure. The residue was triturated with diethyl ether to provide (4-chloro-2-cyclopropyl-5-methyl-pyrimidin-6-yloxy)-acetic acid as a white solid (220 mg, 91%).

¹H nmr (400 MHz, CDCl₃) δ_{H} 12.0 (1H, br s), 4.81 (2H, s), 2.20 (3H, s), 2.10 (1H, m), 1.10 (4H, m) ppm.

### Example 35

### synthesis of 4-amino-5-ethenyl-6-methoxycarbonylmethoxy-2-methylthio-pyrimidine (compound 2-366)

### 35.1 Preparation of 4-chloro-6-hydroxy-5-iodo-2-methylthio-pyrimidine

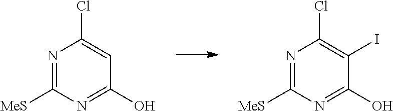

N-Iodosuccinimide (8.1 g, 36 mmol) was added to a stirred solution of 2-chloro-6-hydroxy-2-methylthiopyrimidine (5.28 g, 30 mmol) in methanol (100 ml) and the resulting mixture stirred at ambient temperature for 1 hour. The reaction mixture was filtered and the solid washed with diethyl ether to provide 4-chloro-6-hydroxy-5-iodo-2-methylthiopyrimidine as a white solid (8.0 g, 88%).

¹H nmr (400 MHz, CDCl₃) δ_{H} 2.50 (3H, s) ppm (hydroxy proton not observed).

### 35.2 Preparation of 4,6-dichloro-5-iodo-2-methylthio-pyrimidine

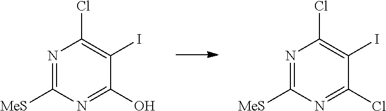

A suspension of 4-chloro-6-hydroxy-5-iodo-2-methylthiopyrimidine (906 mg, 3.0 mmol) in phosphorus oxychloride (6 ml, 60 mmol) was heated at reflux for 3 hours, then allowed to cool and poured into iced water. The mixture was filtered and the solid purified by chromatography on silica using hexane/ethyl acetate (19:1) as eluent to provide 4,6-dichloro-5-iodo-2-methylthio-pyrimidine as a white solid (700 mg, 73%).

¹H nmr (400 MHz, CDCl₃) δ_{H} 2.50 (3H, s) ppm.

### 35.3 Preparation of 4-amino-6-chloro-5-iodo-2-methylthio-pyrimidine

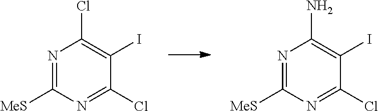

A solution of 4,6-dichloro-5-iodo-2-methylthio-pyrimidine (320 mg, 1.0 mmol) and ammonia (7M in methanol; 0.15 ml, 1.1 mmol) in methanol (2.5 ml) was heated in a microwave reactor at 120° C. for 20 minutes, then cooled to 0° C. The solid was filtered and washed with hexane to provide 4-amino-6-chloro-5-iodo-2-methylthio-pyrimidine as a white solid (210 mg, 70%).

¹H nmr (400 MHz, d₆-DMSO) δ_{F}, 7.20 (2H, br s), 2.50 (3H, s) ppm.

### 35.4 Preparation of 4-amino-5-iodo-6-methoxycarbonylmethoxy-2-methylthio-pyrimidine

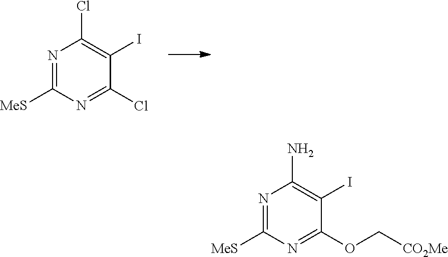

Sodium hydride (50% in mineral oil; 72 mg, 1.5 mmol) was added to a stirred solution of methyl glycolate (99 mg, 1.1 mmol) in tetrahydrofuran (2 ml). After stirring for 10 minutes, a solution of 4-amino-6-chloro-5-iodo-2-methylthio-pyrimidine (301 mg, 1.0 mmol) in tetrahydrofuran was added and the reaction mixture heated at reflux for 2 hours. After cooling, saturated aqueous ammonium chloride was added and the resulting mixture extracted with ethyl acetate. The combined organic extracts were washed with brine, dried over magnesium sulphate, filtered and evaporated under reduced pressure. The residue was purified by chromatography on silica, using a gradient of ethyl acetate in hexane (0-20%) as eluent to provide 4-amino-5-iodo-6-methoxycarbonylmethoxy-2-methylthio-pyrimidine as a yellow solid (130 mg, 36%).

¹H nmr (400 MHz, CDCl₃) δ_{H} 5.30 (2H, br s), 4.88 (2H, s), 3.75 (3H, s), 2.50 (3H, s) ppm.

### 35.5 Preparation of 4-amino-5-ethenyl-6-methoxycarbonylmethoxy-2-methylthio-pyrimidine (compound 2-366)

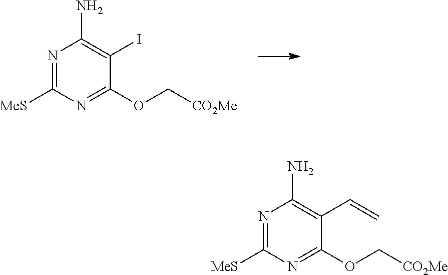

A mixture of vinyl boronic acid pinacol ester (0.067 ml, 0.375 mmol), 4-amino-5-iodo-6-methoxycarbonylmethoxy-2-methylthio-pyrimidine (89 mg, 0.25 mmol), caesium fluoride (76 mg, 0.50 mmol), [1,1′-bis(diphenylphosphino)-ferrocene] dichloropalladium (II) complex with dichloromethane (1:1) (20 mg, 0.025 mmol), dimethoxyethane (1.5 ml) and water (1.5 ml) was heated in a microwave reactor at 150° C. for 20 minutes, then allowed to cool. Dichloromethane was added and the solution washed with brine, dried over magnesium sulphate, filtered and evaporated under reduced pressure. The residue was purified by chromatography on silica using hexane/ethyl acetate (4:1) as eluent to provide 4-amino-5-ethenyl-6-methoxycarbonylmethoxy-2-methylthio-pyrimidine as a brown solid (43 mg, 67%).

¹H nmr (400 MHz, CDCl₃) δ_{H} 6.60 (1H, m), 5.65 (1H, d), 5.50 (1H, d), 5.10 (2H, br s), 4.82 (2H, s), 3.72 (3H, s), 2.40 (3H, s) ppm.

### Example 36

### Pre-Emergence Biological Efficacy

Seeds of Alopecurus myosuroides (ALOMY), Setaria faberi (SETFA), Echinochloa crus-galli (ECHCG), Solanum nigrum (SOLNI), Amaranthus retoflexus (AMARE) and Ipomea hederaceae (IPOHE) were sown in standard soil in pots. After cultivation for one day under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethylene sorbitan monolaurate, CAS RN 9005-64-5) to give a final dose of 250 or 1000 g/ha of test compound.

The test plants were then grown under controlled conditions in the glasshouse (at 24/16° C., day/night; 14 hours light; 65% humidity) and watered twice daily. After 13 days the test was evaluated (100=total damage to plant; 0=no damage to plant). Results are shown below in Table B1.

TABLE B1
Percentage damage caused to weed species by compounds
of the invention when applied pre-emergence.

| Compound | | Species | | | | | |
|---|---|---|---|---|---|---|---|
| Number | Dose (g/ha) | SOLNI | AMARE | IPOHE | SETFA | ALOMY | ECHCG |
| 2-41 | 1,000 | 50 | 70 | 60 | 30 | 20 | 20 |
| 2-61 | 1,000 | 100 | 100 | 100 | 20 | 40 | 80 |
| 2-62 (E) | 1,000 | 70 | 40 | 30 | 0 | 10 | 10 |
| 2-62 (Z) | 1,000 | 100 | 90 | 100 | 20 | 20 | 10 |
| 2-64 | 1,000 | 80 | 100 | 100 | 20 | 20 | 0 |
| 2-65 | 250 | 0 | 0 | 0 | 0 | 0 | 0 |
| 2-66 | 1,000 | 0 | — | 0 | 0 | 0 | 0 |
| 2-69 | 1,000 | 90 | 100 | 70 | 0 | 10 | 0 |
| 2-77 | 250 | 0 | 0 | 0 | 0 | 0 | 0 |
| 2-78 | 1,000 | 90 | 90 | 100 | 10 | 20 | 30 |
| 2-221 | 1,000 | 60 | 60 | 70 | 0 | 30 | 0 |
| 2-225 | 1,000 | 0 | 40 | 20 | 0 | 0 | 0 |
| 2-362 | 1,000 | 10 | 40 | 30 | 40 | 40 | 10 |
| 2-366 | 1,000 | 0 | 0 | 0 | 0 | 0 | 0 |
| 2-375 | 1,000 | 0 | 0 | 20 | 0 | 0 | 0 |
| 2-376 | 1,000 | 0 | 30 | 0 | 0 | 0 | 0 |
| 2-389 | 1,000 | 0 | 20 | 0 | 0 | 0 | 0 |
| 2-398 | 1,000 | 90 | 90 | 100 | 0 | 0 | 0 |
| 2-399 | 1,000 | 0 | 0 | 0 | 0 | 0 | 0 |
| 2-400 | 1,000 | 100 | 90 | 60 | 0 | 0 | 0 |
| 2-401 | 1,000 | 10 | 70 | 70 | 0 | 0 | 0 |
| 2-402 | 1,000 | 0 | 0 | 0 | 0 | 0 | 0 |
| 5-41 | 1,000 | 30 | 100 | 10 | 30 | 20 | 30 |
| 5-61 | 1,000 | 90 | 100 | 90 | 60 | 60 | 60 |
| 6-61 | 1,000 | 90 | 100 | 80 | 30 | 70 | 50 |
| 9-61 | 1,000 | 70 | 90 | 10 | 0 | 0 | 30 |
| 14-61 | 1,000 | 40 | 60 | 10 | 10 | 10 | 20 |
| 15-61 | 1,000 | 100 | 100 | 100 | 70 | 80 | 70 |
| 19-61 | 1,000 | 100 | 100 | 100 | 30 | 50 | 70 |
| 26-61 | 1,000 | 80 | 100 | 20 | 0 | 0 | 0 |
| 27-21 | 1,000 | 100 | 100 | 100 | 100 | 90 | 100 |
| 27-41 | 1,000 | 80 | 50 | 90 | 10 | 30 | 20 |
| 27-53 | 1,000 | 100 | 90 | 100 | 10 | 20 | 20 |
| 27-58 | 1,000 | 0 | — | 10 | 0 | 0 | 0 |
| 27-61 | 1,000 | 100 | 100 | 90 | 90 | 90 | 90 |
| 27-62 (E) | 1,000 | 80 | 50 | 90 | 80 | 90 | 90 |
| 27-62 (Z) | 1,000 | 100 | 100 | 100 | 80 | 80 | 90 |
| 27-63 | 1,000 | 100 | 100 | 100 | 50 | 50 | 80 |
| 27-67 | 1,000 | 100 | 30 | 40 | 10 | 20 | 20 |
| 27-69 | 1,000 | 100 | 100 | 30 | 60 | 40 | 30 |
| 27-398 | 1,000 | 0 | 0 | 0 | 0 | 0 | 0 |
| 30-61 | 1,000 | 20 | 0 | 0 | 0 | 0 | 0 |
| 31-372 | 1,000 | 0 | 0 | 0 | 0 | 0 | 0 |
| 31-42 | 1,000 | 0 | 0 | 0 | 0 | 0 | 0 |
| 31-53 | 1,000 | 100 | 100 | 90 | 90 | 80 | 90 |
| 31-61 | 1,000 | 0 | 10 | 0 | 0 | 0 | 0 |
| 31-62 | 1,000 | 0 | 20 | 0 | 0 | 0 | 0 |
| 31-63 | 1,000 | 0 | 0 | 0 | 0 | 0 | 0 |
| 31-64 | 1,000 | 0 | 0 | 0 | 0 | 0 | 0 |
| 31-67 | 1,000 | 0 | 0 | 0 | 0 | 0 | 0 |
| 33-21 | 1,000 | 100 | 100 | 80 | 100 | 90 | 100 |
| 33-41 | 1,000 | 30 | 20 | 0 | 30 | 10 | 10 |
| 33-53 | 1,000 | 0 | 0 | 0 | 0 | 0 | 0 |
| 33-58 | 1,000 | 0 | 0 | 0 | 0 | 0 | 0 |
| 33-61 | 1,000 | 30 | 50 | 40 | 10 | 0 | 0 |
| 33-62 (E) | 1,000 | 80 | 100 | 0 | 80 | 70 | 90 |
| 33-62 (Z) | 1,000 | 60 | 70 | 20 | 30 | 30 | 30 |
| 33-64 | 1,000 | 0 | 0 | 0 | 0 | 0 | 0 |
| 33-67 | 1,000 | 0 | 0 | 10 | 0 | 0 | 0 |
| 33-101 | 1,000 | 90 | 100 | 30 | 90 | 60 | 90 |
| 33-306 | 1,000 | 80 | 40 | 0 | 20 | 20 | 20 |
| 33-316 | 1,000 | 80 | 90 | 10 | 70 | 60 | 90 |
| 33-326 | 1,000 | 60 | 60 | 10 | 20 | 20 | 40 |
| 33-336 | 1,000 | 80 | 100 | 20 | 80 | 70 | 90 |
| 33-391 | 1,000 | 100 | 100 | 20 | 90 | 70 | 100 |
| 33-392 | 1,000 | 80 | 100 | 0 | 90 | 70 | 100 |
| 33-393 | 1,000 | 20 | 20 | 0 | 0 | 0 | 0 |
| 33-394 | 1,000 | 40 | 40 | 0 | 30 | 20 | 30 |
| 33-395 | 1,000 | 100 | 100 | 30 | 80 | 60 | 90 |
| 33-396 | 1,000 | 90 | 100 | 50 | 80 | 70 | 90 |
| 33-397 | 1,000 | 100 | 100 | 50 | 90 | 80 | 100 |
| 36-61 | 1,000 | 100 | 100 | 100 | 80 | 70 | 90 |
| 41-61 | 1,000 | 0 | 100 | 40 | 10 | 20 | 10 |
| 42-61 | 1,000 | 20 | 60 | 0 | 20 | 10 | 10 |
| 43-61 | 1,000 | 70 | 60 | 10 | 50 | 30 | 70 |
| 44-61 | 1,000 | 40 | 50 | 10 | 30 | 20 | 40 |
| 45-61 | 1,000 | 40 | 100 | 0 | 50 | 50 | 40 |
| 47-61 | 1,000 | 0 | 0 | 0 | 0 | 0 | 0 |
| 60-41 | 1,000 | 40 | 0 | 10 | 10 | 20 | 10 |
| 61-61 | 1,000 | 30 | 0 | 0 | 0 | 0 | 0 |
| 62-61 | 1,000 | 100 | 100 | 100 | 0 | 0 | 0 |
| 63-61 | 1,000 | 0 | 0 | 0 | 0 | 0 | 0 |
| 64-61 | 1,000 | 10 | 30 | 0 | 10 | 10 | 10 |
| 65-53 | 1,000 | 0 | 0 | 20 | 0 | 0 | 0 |
| 66-53 | 1,000 | 20 | 10 | 30 | 0 | 0 | 0 |
| 67-61 | 1,000 | 70 | 100 | 50 | 30 | 30 | 30 |
| 68-61 | 250 | 30 | 10 | 0 | 0 | 0 | 0 |
| 69-61 | 1,000 | 0 | 0 | 0 | 0 | 0 | 0 |
| 70-61 | 1,000 | 100 | 100 | 80 | 70 | 80 | 90 |
| 71-61 | 1,000 | 40 | 0 | 0 | 0 | 10 | 20 |
| 72-61 | 1,000 | 50 | 60 | 70 | 0 | 0 | 0 |
| 73-61 | 1,000 | 0 | 0 | 0 | 0 | 0 | 0 |

### Example 37

### Post-Emergence Biological Efficacy

Seeds of Alopecurus myosuroides (ALOMY), Setaria faberi (SETFA), Echinochloa crus-galli (ECHCG), Solanum nigrum (SOLNI), Amaranthus retoflexus (AMARE) and Ipomea hederaceae (IPOHE) were sown in standard soil in pots. After cultivation for 8 days under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethylene sorbitan monolaurate, CAS RN 9005-64-5) to give a final dose of 250 or 1000 g/ha of test compound.

The test plants were then grown on under controlled conditions in a glasshouse (at 24/16° C., day/night; 14 hours light; 65% humidity) and watered twice daily. After 13 days the test was evaluated (100=total damage to plant; 0=no damage to plant). Results are shown below in Table B2.

TABLE B2
Percentage damage caused to weed species by compounds
of the invention when applied post-emergence

| Compound | | Species | | | | | |
|---|---|---|---|---|---|---|---|
| Number | Dose (g/ha) | SOLNI | AMARE | IPOHE | SETFA | ALOMY | ECHCG |
| 2-41 | 1,000 | 90 | 100 | 100 | 80 | 40 | 60 |
| 2-61 | 1,000 | 100 | 100 | 100 | 80 | 40 | 60 |
| 2-62 (E) | 1,000 | 70 | 40 | 50 | 20 | 10 | 10 |
| 2-62 (Z) | 1,000 | 70 | 70 | 100 | 30 | 10 | 10 |
| 2-64 | 1,000 | 80 | 60 | 70 | 30 | 10 | 40 |
| 2-65 | 250 | 40 | 20 | 40 | 0 | 0 | 0 |
| 2-66 | 1,000 | 60 | 50 | 40 | 10 | 0 | 0 |
| 2-69 | 1,000 | 80 | 20 | 70 | 10 | 10 | 0 |
| 2-77 | 250 | 40 | 30 | 70 | 0 | 0 | 0 |
| 2-78 | 1,000 | 100 | 100 | 100 | 60 | 30 | 60 |
| 2-221 | 1,000 | 80 | 100 | 80 | 60 | 20 | 70 |
| 2-225 | 1,000 | 50 | 40 | 30 | 0 | 0 | 0 |
| 2-362 | 1,000 | 80 | 80 | 80 | 70 | 40 | 60 |
| 2-366 | 1,000 | 20 | 0 | 0 | 0 | 0 | 0 |
| 2-375 | 1,000 | 10 | 10 | 20 | 0 | 0 | 0 |
| 2-376 | 1,000 | 30 | 20 | 50 | 20 | 0 | 0 |
| 2-389 | 1,000 | 30 | 0 | 20 | 0 | 0 | 0 |
| 2-398 | 1,000 | 80 | 90 | 90 | 0 | 0 | 0 |
| 2-399 | 1,000 | 20 | 20 | 20 | 0 | 0 | 0 |
| 2-400 | 1,000 | 100 | 80 | 60 | 0 | 0 | 0 |
| 2-401 | 1,000 | 80 | 70 | 70 | 0 | 0 | 0 |
| 2-402 | 1,000 | 20 | 0 | 50 | 0 | 0 | 0 |
| 5-41 | 1,000 | 70 | 100 | 40 | 30 | 30 | 20 |
| 5-61 | 1,000 | 100 | 100 | 100 | 100 | 80 | 90 |
| 6-61 | 1,000 | 90 | 100 | 70 | 30 | 60 | 50 |
| 9-61 | 1,000 | 90 | 100 | 70 | 0 | 20 | 20 |
| 14-61 | 1,000 | 80 | 10 | 40 | 0 | 0 | 0 |
| 15-61 | 1,000 | 100 | 100 | 80 | 70 | 90 | 70 |
| 19-61 | 1,000 | 100 | 100 | 100 | 40 | 40 | 60 |
| 26-61 | 1,000 | 80 | 100 | 70 | 0 | 50 | 60 |
| 27-21 | 1,000 | 90 | 100 | 90 | 100 | 90 | 90 |
| 27-41 | 1,000 | 80 | 100 | 60 | 80 | 30 | 50 |
| 27-53 | 1,000 | 90 | 100 | 60 | 40 | 30 | 30 |
| 27-58 | 1,000 | 80 | 90 | 60 | 40 | 10 | 20 |
| 27-61 | 1,000 | 80 | 100 | 80 | 100 | 90 | 90 |
| 27-62 (E) | 1,000 | 100 | 100 | 90 | 100 | 90 | 100 |
| 27-62 (Z) | 1,000 | 80 | 100 | 100 | 90 | 70 | 80 |
| 27-63 | 1,000 | 90 | 100 | 90 | 90 | 60 | 70 |
| 27-67 | 1,000 | 80 | 40 | 60 | 10 | 0 | 10 |
| 27-69 | 1,000 | 80 | 90 | 40 | 40 | 40 | 10 |
| 27-398 | 1,000 | 20 | 0 | 40 | 0 | 0 | 0 |
| 30-61 | 1,000 | 80 | 10 | 10 | 0 | 0 | 0 |
| 31-42 | 1,000 | 10 | 0 | 0 | 0 | 0 | 0 |
| 31-53 | 1,000 | 100 | 100 | 90 | 100 | 90 | 100 |
| 31-61 | 1,000 | 70 | 70 | 10 | 0 | 20 | 0 |
| 31-62 | 1,000 | 50 | 30 | 50 | 10 | 0 | 0 |
| 31-63 | 1,000 | 50 | 40 | 40 | 0 | 0 | 0 |
| 31-64 | 1,000 | 60 | 40 | 60 | 20 | 0 | 0 |
| 31-67 | 1,000 | 80 | 50 | 80 | 0 | 0 | 0 |
| 31-372 | 1,000 | 30 | 20 | 30 | 30 | 10 | 10 |
| 33-21 | 1,000 | 90 | 100 | 70 | 100 | 90 | 100 |
| 33-41 | 1,000 | 70 | 100 | 10 | 70 | 30 | 50 |
| 33-53 | 1,000 | 50 | 100 | 40 | 20 | 10 | 20 |
| 33-58 | 1,000 | 10 | 10 | 0 | 60 | 10 | 10 |
| 33-61 | 1,000 | 80 | 70 | 90 | 30 | 20 | 20 |
| 33-62 (Z) | 1,000 | 80 | 100 | 40 | 80 | 80 | 80 |
| 33-62 (Z) | 1,000 | 80 | 100 | 30 | 70 | 60 | 70 |
| 33-64 | 1,000 | 20 | 10 | 10 | 0 | 0 | 0 |
| 33-67 | 1,000 | 40 | 10 | 20 | 0 | 0 | 0 |
| 33-101 | 1,000 | 90 | 100 | 60 | 80 | 80 | 90 |
| 33-306 | 1,000 | 100 | 100 | 30 | 70 | 40 | 70 |
| 33-316 | 1,000 | 90 | 100 | 40 | 90 | 70 | 100 |
| 33-326 | 1,000 | 90 | 100 | 50 | 40 | 20 | 80 |
| 33-336 | 1,000 | 90 | 100 | 40 | 90 | 90 | 90 |
| 33-391 | 1,000 | 80 | 100 | 80 | 90 | 70 | 90 |
| 33-392 | 1,000 | 90 | 100 | 80 | 60 | 70 | 80 |
| 33-393 | 1,000 | 70 | 70 | 70 | 0 | 0 | 20 |
| 33-394 | 1,000 | 80 | 100 | 30 | 50 | 30 | 60 |
| 33-395 | 1,000 | 100 | 100 | 30 | 100 | 70 | 100 |
| 33-396 | 1,000 | 90 | 100 | 60 | 90 | 70 | 100 |
| 33-397 | 1,000 | 90 | 100 | 80 | 90 | 80 | 90 |
| 36-61 | 1,000 | 80 | 100 | 90 | 80 | 70 | 90 |
| 41-61 | 1,000 | 90 | 100 | 60 | 10 | 20 | 10 |
| 42-61 | 1,000 | 30 | 100 | 10 | 40 | 30 | 30 |
| 43-61 | 1,000 | 70 | 20 | 10 | 20 | 30 | 30 |
| 44-61 | 1,000 | 90 | 100 | 30 | 60 | 40 | 50 |
| 45-61 | 1,000 | 70 | 100 | 30 | 50 | 50 | 40 |
| 47-61 | 1,000 | 70 | 100 | 10 | 10 | 10 | 10 |
| 60-41 | 1,000 | 100 | 100 | 40 | 10 | 40 | 30 |
| 61-61 | 1,000 | 20 | 10 | 0 | 0 | 0 | 0 |
| 62-61 | 1,000 | 90 | 100 | 90 | 10 | 0 | 0 |
| 63-61 | 1,000 | 20 | 0 | 20 | 0 | 0 | 0 |
| 64-61 | 1,000 | 60 | 100 | 10 | 30 | 10 | 10 |
| 65-53 | 1,000 | 70 | 20 | 50 | 10 | 0 | 10 |
| 66-53 | 1,000 | 60 | 20 | 30 | 10 | 0 | 0 |
| 67-61 | 1,000 | 90 | 100 | 60 | 10 | 20 | 40 |
| 68-61 | 250 | 80 | 30 | 40 | 0 | 0 | 0 |
| 69-61 | 1,000 | 20 | 0 | 30 | 0 | 0 | 0 |
| 70-61 | 1,000 | 90 | 100 | 80 | 40 | 100 | 100 |
| 71-61 | 1,000 | 60 | 20 | 40 | 0 | 0 | 30 |
| 72-61 | 1,000 | 60 | 10 | 50 | 0 | 10 | 0 |
| 73-61 | 1,000 | 30 | 20 | 0 | 0 | 0 | 0 |

1. A compound of formula (I) 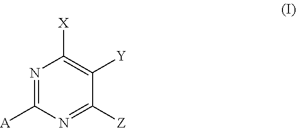
or salt or N-oxide thereof,
wherein:
A is halogen, optionally substituted alkylthio, optionally substituted alkyl, optionally substituted alkenyl or an optionally substituted 3-8 membered carbocyclic ring;
X is azido, halogen, optionally substituted alkoxy, optionally substituted alkylthio, optionally substituted alkylsulphinyl, optionally substituted alkylsulphonyl or NR⁵R⁶;
R⁵ is hydrogen, C₂₋₄ alkenyl, SO₂R^{ss}, C(O)R^{uu} or optionally substituted C₁₋₄ alkyl;
R⁶ is hydrogen, C₂₋₄ alkenyl or optionally substituted C₁₋₄;
each R^{ss} is independently C₁₋₄ alkyl or phenyl optionally substituted by 1-3 groups R^{zz};
each R^{uu} is independently C₁₋₄ alkyl, phenyl optionally substituted by 1-3 groups R^{zz}, C₁₋₄ alkoxy, or NR^{ac}R^{ad};
each R^{zz} is independently halogen, C₁₋₄ alkyl, C₁₋₄ alkoxy, or C₁₋₄ alkylsulphonyl;
R^{ac} and R^{ad} are each independently hydrogen or C₁₋₄ alkyl;
or R⁵ and R⁶ together form a group ═C(Rⁱ)OR^{j}, ═C(R^{k})SR^{l}, ═C(R^{m})NRⁿR^{o} wherein
Rⁱ is hydrogen, C₁₋₄ alkyl, C₃₋₆ cycloalkyl, phenyl, C₁₋₄ alkoxy, C₁₋₄ alkylthio, or NR^{ac}R^{ad} wherein R^{ac} and R^{ad} are as defined above,
R^{j} and R^{l} are each independently C₁₋₄ alkyl,
R^{k} is hydrogen, C₁₋₄ alkyl, C₃₋₆ cycloalkyl, phenyl, C₁₋₄ alkylthio, or NR^{ac}R^{ad} wherein R^{ac} and R^{ad} are as defined above,
R^{m} is hydrogen, C₁₋₄ alkyl, C₃₋₆ cycloalkyl, phenyl, or NR^{ac}R^{ad} wherein R^{ac} and R^{ad} are as defined above, and
Rⁿ and R^{o} are each independently hydrogen or C₁₋₄alkyl;
Y is optionally substituted alkyl, optionally substituted cycloalkyl, optionally substituted alkenyl, or optionally substituted alkynyl;
and,
Z is Oₘ—(CHR^{w})ₙ—C(O)R^{cb}, wherein
m is an integer of 0 or 1,
n is an integer of 0 or 1 and n≧m,
R^{w} is hydrogen or C₁₋₄ alkyl
R^{cb} is hydroxy, optionally substituted alkylthio, NH₂, or OR^{co},
R^{co} is C₁₋₂₀ alkyl optionally substituted by 1-3 groups R^{cq}, or C₁₋₂₀ haloalkyl optionally substituted by 1-3 groups R^{cq};
each R^{cq} is independently C₁₋₆ alkoxy, phenyl optionally substituted by 1-3 groups R^{cr}, or heteroaryl optionally substituted by 1-2 groups R^{cs};
each R^{cr} and each R^{cs} are independently halogen, cyano, C₁₋₄ alkyl, C₁₋₄ haloalkyl, C₂₋₆ alkoxyalkyl, C₁₋₄ alkoxy, C₁₋₄ haloalkoxy, C₁₋₄alkylsulphonyl, or C₁₋₄ alkoxycarbonyl;
provided that:
i) when Y is C₁₋₄ alkyl, C₁₋₄ haloalkyl, C₂₋₄ alkoxyalkyl, C₂₋₄ alkylthioalkyl, C₂₋₄ alkenyl, C₂₋₄ haloalkenyl, C₂₋₄ alkoxyalkenyl, C₂₋₄ thioalkylalkenyl, C₂₋₄ alkynyl, C₂₋₄ haloalkynyl, C₂₋₄ alkylcarbonyl or C₂₋₄ haloalkylcarbonyl;
X is NR⁵R⁶;
R⁵ is H, C₁₋₄ alkyl, C₃₋₄ alkenyl, C₁₋₄ alkylsulfonyl or C₁₋₄ acyl;
R⁶ is H, C₁₋₄ alkyl or C₃₋₄ alkenyl;
A is C1-C6 alkyl, cyclopropyl, C1-C6 haloalkyl, C2-C6 alkenyl, C2-C6 haloalkenyl or a group of the formula 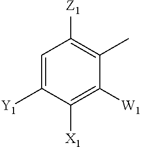
W₁ represents H or halogen;
X₁ represents H, halogen, nitro, cyano, formyl, C₁₋₆ alkyl, C₂₋₆ alkenyl, C₂₋₆ alkynyl, C₁₋₆ alkoxy, C₂₋₄ alkoxyalkyl, C₁₋₆ alkylcarbonyl, C₁₋₆ alkylthio, C₁₋₆ alkylsulfinyl, C₁₋₆ alkylsulfonyl, C₂₋₄ alkenyloxy, C₂₋₄ alkynyloxy, C₂₋₄ alkenylthio, C₂₋₄ alkynylthio, C₁₋₆ haloalkyl, C₂₋₆ halo-alkenyl, C₂₋₆ haloalkynyl, C₁₋₆ haloalkoxy, C₂₋₄ haloalkoxyalkyl, C₂₋₆ haloalkylcarbonyl, C₁₋₆ haloalkylthio, C₁₋₆ haloalkylsulfinyl, C₁₋₆ halo-alkylsulfonyl, C₃₋₆ trialkylsilyl, C₂₋₄ haloalkenyloxy, C₂₋₄ haloalkynyloxy, C₂₋₄ haloalkenylthio, C₂₋₄ haloalkynylthio, —C(O)OR^{7′}, —C(O)NR^{6′}R^{7′}, —CR^{6′}NOR^{7′}, —NR^{6′}R^{7′}, —NR^{6′}OR^{7′}, —NR^{6′}SO2R^{7′}, —NR^{6′}C(O)R^{7′}, —NR^{6′}C(O)OR^{7′}, —NR^{6′}C(O)NR^{6′}R^{7′} or —NCR^{6′}NR^{6′}R^{7′};
R^{6′} represents H, C₁₋₄ alkyl or C₁₋₄ haloalkyl;
R^{7′} represents C₁₋₄ alkyl or C₁₋₄ haloalkyl;
Y₁ represents H, halogen, C₁₋₆ alkyl, C₁₋₆ haloalkyl, C₁₋₆ alkoxy, C₁₋₆ haloalkoxy, C₂₋₆ alkenyl or C₂₋₆ haloalkenyl, or, X₁ and Y₁ taken together, represents —O(CH₂)ₙₙCH₂—, or —O(CH₂)ₙₙO— wherein nn=1 or 2; and
Z₁ represents H or halogen, then
Z is other than CO₂H, CO₂Me, CO₂Et, CO₂ⁿBu or CO₂⁻NHEt₃⁺.

2. The compound according to claim 1, wherein
A is halogen, C₁₋₄ alkylthio, phenyl optionally substituted by 1-3 groups R¹, or C₃₋₆ cycloalkyl optionally substituted by 1-4 groups R²;
R¹ is halogen, cyano, C₁₋₄ alkyl, C₁₋₄ haloalkyl, C₁₋₄ alkoxy, C₁₋₄ haloalkoxy, C₁₋₄ alkylthio, C₁₋₄ haloalkylthio, amino, C₁₋₄ alkylamino, di(C₁₋₄)alkylamino or two adjacent groups R¹ together with the atoms to which they are joined form a 6-membered aromatic ring, said ring being optionally substituted by 1-2 groups selected from: halogen, cyano, C₁₋₄ alkyl, C₁₋₄ haloalkyl, C₁₋₄ alkoxy, C₁₋₄ haloalkoxy, C₁₋₄ alkylthio, C₁₋₄ haloalkylthio;
R² is halogen, cyano, C₁₋₄ alkyl, C₁₋₄ haloalkyl, C₃₋₆ cycloalkyl, C₁₋₄ alkoxy, C₁₋₄haloalkoxy, C₁₋₄ alkoxycarbonyl, or C₁₋₄ alkylaminocarbonyl; or any two geminal R² groups together form a group selected from oxo, ═CR^{mm}Rⁿⁿ, or ═NOR^{oo}; or two groups R² together with the atoms to which they are joined form a 3-6-membered ring system, said ring system being optionally substituted by 1-2 groups selected from: halogen, cyano, C₁₋₄ alkyl, C₁₋₄ haloalkyl, C₁₋₄ alkoxy, or C₁₋₄ haloalkoxy;
R^{mm} and Rⁿⁿ are each independently hydrogen, halogen, cyano, nitro, C₁₋₄ alkyl, or C₁₋₄ alkoxycarbonyl;
R^{oo} is hydrogen, C₁₋₄ alkyl, C₃₋₆cycloalkyl (C₁₋₂)alkyl or C₃₋₆cycloalkyl;
X is azido, halogen, C₁₋₃ alkoxy, C₁₋₄ alkoxycarbonylC₁₋₃alkoxy, C₁₋₄ alkylthio, C₁₋₄ alkylsulphinyl, C₁₋₄ alkylsulphonyl or NR⁵R⁶ wherein
R⁵ is hydrogen, C₁₋₄ alkyl optionally substituted with 1 or 2 C₁₋₄ alkoxy groups, C₁₋₄ haloalkyl optionally substituted with 1 or 2 C₁₋₄ alkoxy groups, C₂₋₄ alkenyl, SO₂R^{ss}, or C(O)R^{uu};
R⁶ is hydrogen, C₁₋₄ alkyl optionally substituted with 1 or 2 C₁₋₄ alkoxy groups, or C₁₋₄ haloalkyl optionally substituted with 1 or 2 C₁₋₄ alkoxy groups, C₂₋₄ alkenyl;
R^{ss} and R^{uu} are C₁₋₃ alkyl,
Y is C₁₋₆ alkyl optionally substituted by 1-3 groups R^{ba}, C₁₋₆haloalkyl optionally substituted by 1-3 groups R^{ba}, C₃₋₆cycloalkyl optionally substituted by 1-3 groups R^{bc}, C₂₋₆alkenyl optionally substituted by 1-3 groups R^{bd}, C₂₋₆ alkynyl optionally substituted by 1-3 groups R^{be}, wherein
each R^{ba} is independently cyano, nitro, hydroxyl, C₃₋₆cycloalkyl, C₁₋₄ alkoxy, C₁₋₄alkylthio, C₁₋₄alkylcarbonyl or C₁₋₄alkoxycarbonyl, or two geminal R^{ba} together form an oxo or oximino group;
R^{bc} is halogen, cyano, C₁₋₄alkyl, C₁₋₄alkoxy, or C₁₋₄alkoxycarbonyl;
R^{bd} is halogen, cyano, C₃₋₆cycloalkyl, C₁₋₄alkylcarbonyl, or C₁₋₄ alkoxycarbonyl;
R^{be} is halogen, cyano, hydroxyl, C₁₋₄alkoxycarbonyl, or C₃₋₁₂ trialkylsilyl;
and,
Z is Oₘ—(CH₂)ₙ—C(O)R^{cb}, wherein n is an integer of 0 or 1, m is an integer of 0 or 1, and n=m
R^{cb} is hydroxyl, C₁₋₁₀ alkoxy, phenyl C₁₋₂ alkoxy or NH₂.

3. The compound according to claim 1 or claim 2, wherein
A is Cl, phenyl optionally substituted by 1-3 groups R¹, cyclopropyl optionally substituted by 1-2 groups R²;
each R¹ is independently halogen, cyano, C₁₋₂ alkyl, C₁₋₂ haloalkyl, C₁₋₂ alkoxy, C₁₋₂ haloalkoxy, amino, C₁₋₄ alkylamino, or di(C₁₋₄)alkylamino;
each R² is independently halogen, cyano, C₁₋₂alkyl, C₁₋₂ haloalkyl, C₁₋₂ alkoxy, C₁₋₂ haloalkoxy or C₂₋₄ alkoxycarbonyl;
X is N₃, Cl, OCH₃, OCH₂CO₂CH₃, NH₂, NHCH₃, N(CH₃)₂, NH-i-propyl, SMe, SOMe, SO₂Me, NHCOCH₃, NHC(O)OCH₃, NHSO₂CH₃, NCH₃COCH₃, NCH₃C(O)OCH₃, or NCH₃SO₂CH₃;
Y is C₁₋₃ alkyl, C₁₋₃ haloalkyl, C₂₋₅alkoxyalkyl, cyclopropyl optionally substituted by 1 or 2 groups R^{bc}, C₂₋₄ alkenyl, C₂₋₄ haloalkenyl, or C₂₋₄ alkynyl optionally substituted by 1 or 2 groups R^{be}, wherein
each R^{bc} is independently halogen or C₁₋₂ alkyl, and each R^{be} is independently halogen or C₃₋₉ trialkylsilyl;
and
Z is selected from the group consisting of CO₂H, CO₂CH₃, CO₂CH₂CH₃, CO₂-i-propyl, CO₂-n-propyl, CO₂CH₂-i-propyl, CO₂CH₂-Phenyl, CONH₂, OCH₂CO₂H, OCH₂CO₂CH₃.

4. The compound according to any one of the preceding claims wherein
A is selected from the group consisting of:
Cl, methylthio, isopropyl, cyclopropyl, 2-methylcyclopropyl, 4-methylphenyl, 4-methoxyphenyl, 4-chlorophenyl, 4-bromophenyl, 4-iodophenyl, 4-fluorophenyl, 4-trifluoromethylphenyl, 4-trifluoromethoxyphenyl, 2,4-dimethoxyphenyl, 2,4-dichlorophenyl, 2-chloro-4-methylphenyl, 2-chloro-4-trifluoromethylphenyl, 2-fluoro-4-methylphenyl, 2-fluoro-3-methoxyphenyl, 2-fluoro-4-methoxyphenyl, 2,4-bis(trifluoromethyl)phenyl, 3,4-dimethylphenyl, 3,4-dimethoxyphenyl, 3-chloro-4-methylphenyl, 3-chloro-4-methoxyphenyl, 3,4-dichlorophenyl, 3-chloro-4-fluorophenyl, 3-chloro-4-trifluoromethylphenyl, 3-fluoro-4-methylphenyl, 3-fluoro-4-methoxyphenyl, 4-methyl-3-nitrophenyl, 4-methoxy-2-methylphenyl, 4-chloro-2-methylphenyl, 4-chloro-3-methylphenyl, 4-chloro-2-methoxyphenyl, 4-chloro-3-methoxyphenyl, 4-chloro-3-nitrophenyl, 4-chloro-3-cyanophenyl, 4-chloro-2-fluorophenyl, 4-chloro-3-fluorophenyl, 4-chloro-2-trifluoromethylphenyl, 4-chloro-3-trifluoromethylphenyl, 4-fluoro-3-methylphenyl, 4-fluoro-3-methoxyphenyl, 4-fluoro-3-trifluoromethylphenyl, 2,4,5-trimethylphenyl, 2,3,4-trimethoxyphenyl, 2,3,4-trichlorophenyl, 2,4,5-trichlorophenyl, 2,4,6-trichlorophenyl, 2,3,4-trifluorophenyl, 2,4-dichloro-3-fluorophenyl, 3,4-dichloro-2-fluorophenyl, 4-chloro-2,3-difluorophenyl, 4-chloro-2,6-difluorophenyl, 4-chloro-3,5-difluorophenyl, 2,4-dichloro-6-fluorophenyl, 4-chloro-2-fluoro-3-methoxyphenyl, 4-chloro-2-fluoro-3-trifluoromethylphenyl, 4-chloro-3-dimethylamino-2-fluorophenyl, and 2-fluoro-3-methoxy-4-methylphenyl;
Y is selected from the group consisting of:
methyl, ethyl, isopropyl, n-propyl, prop-1-en-2-yl, prop-1-enyl, prop-2-enyl, but-1-enyl, pent-1-enyl, difluoromethyl, trifluoromethyl, hydroxymethyl, 1-hydroxyethyl, 2-hydroxyethyl, 1-methylprop-1-enyl, 2-methylprop-1-enyl, 1,2-dimethylprop-1-enyl, 3-methylbut-1-ynyl, 3-methylbut-2-enyl, 3,3-dimethylbut-1-ynyl, acetyl, formyl, methoxymethyl, 2-methoxyethyl, hydroxyiminomethyl, methoxyiminomethyl, 1-(hydroxyimino)ethyl, 1-(methoxyimino)ethyl, cyclopropyl, 1-methylcyclopropyl, 2,2-dichlorocyclopropyl, vinyl, 2-cyclopropylvinyl, 1-ethoxyvinyl, 2,2-dichlorovinyl, ethynyl, prop-1-ynyl, 2-bromoethynyl, 2-chloroethynyl, and 2-trimethylsilylethynyl.

5. A compound according to any one of claim 1, 2 or 4 wherein A is optionally substituted alkylthio.

6. A compound according to any preceding claim wherein X is azido, halogen, optionally substituted alkoxy, optionally substituted alkylthio, optionally substituted alkylsulphinyl or optionally substituted alkylsulphonyl.

7. A compound according to any preceding claim wherein n is 1.

8. A herbicidal composition comprising a compound as defined in any one of the preceding claims and at least one agriculturally acceptable formulation adjuvant or diluent.

9. The herbicidal composition according to claim 8, further comprising a crop safener.

10. A compound as defined in any one of claims 1 to 7, or a herbicidal composition according to claim 8 or claim 9, in admixture with at least one active ingredient selected from the group consisting of: an insecticide, an acaricide, a nematocide, a molluscicide, an herbicide, a fungicide, and a plant growth regulator.

11. Use of a compound according to any one of claims 1 to 7 as a herbicide.

12. A method of controlling weeds in crops of useful plants which comprises applying to said weeds or to the locus of said weeds, or to said crop of useful plants, a compound as defined in any one of claims 1 to 7, or a herbicidal composition according to claim 8 or claim 9, or a mixture according to claim 10.

13. A compound of formula (II) 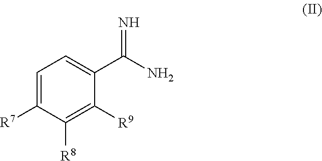
wherein
R⁷ is methyl, Br, or Cl;
R⁸ is H, F, Cl, OR¹⁰, or N(R¹⁰)₂;
R⁹ is H, F, or Cl; and
each R¹⁰ is independently H or C₁₋₄alkyl,
provided that
(i) R⁸ and R⁹ are not both hydrogen,
(ii) when R⁷ is methyl and R⁹ is hydrogen, then R⁸ is not F, Cl, or NH₂,
(iii) when R⁷ is Cl and R⁸ is hydrogen, R⁹ is not Cl,
(iv) when R⁷ is Cl and R⁸ is Cl, R⁹ is not hydrogen, and
(v) when R⁷ is Br and R⁹ is hydrogen, R⁸ is not F.

14. A process for the preparation of a compound of formula (T) 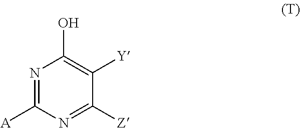
or salt or N-oxide thereof,
wherein:
A is halogen, optionally substituted alkylthio, optionally substituted alkyl, optionally substituted alkenyl or an optionally substituted 3-8 membered carbocyclic ring;
Y′ is hydrogen, optionally substituted alkyl, optionally substituted cycloalkyl, optionally substituted alkenyl, or optionally substituted alkynyl;
and,
Z′ is hydroxyl or CHR^{w})ₙ—C(O)R^{cb}, wherein
n is an integer of 0 or 1
R^{w} is hydrogen or C₁₋₄ alkyl
R^{cb} is hydroxy, optionally substituted alkylthio, NH₂, or OR^{co},
R^{co} is C₁₋₂₀ alkyl optionally substituted by 1-3 groups R^{cq}, or C₁₋₂₀ haloalkyl optionally substituted by 1-3 groups R^{cq};
each R^{cq} is independently C₁₋₆ alkoxy, phenyl optionally substituted by 1-3 groups R^{cr}, or heteroaryl optionally substituted by 1-2 groups R^{cs};
each R^{cr} and each R^{cs} are independently halogen, cyano, C₁₋₄ alkyl, C₁₋₄ haloalkyl, C₂₋₆ alkoxyalkyl, C₁₋₄ alkoxy, C₁₋₄ haloalkoxy, C₁₋₄ alkylsulphonyl, or C₁₋₄ alkoxycarbonyl comprising reacting an amidine of formula (N) 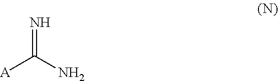
wherein A is as defined above, or a salt form thereof, with a keto ester of the formula (U) 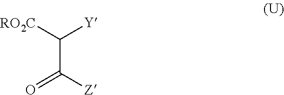
or a salt form thereof,
wherein Y′ and Z′ are as defined above, or Z′ is OR, and R is selected from hydrogen or alkyl.

15. A process according to claim 14 comprising the further step or steps of converting the compound of formula (T) to a compound of formula (I) 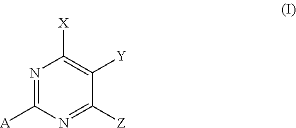
wherein A, Z and Y are as defined in claim 14, and
X is azido, halogen, optionally substituted alkoxy, optionally substituted alkylthio, optionally substituted alkylsulphyl, optionally substituted alkylsulphonyl or NR⁵R⁶;
R⁵ is hydrogen, C₂₋₄ alkenyl, SO₂R^{ss}, C(O)R^{uu} or optionally substituted C₁₋₄ alkyl;
R⁶ is hydrogen, C₂₋₄ alkenyl or optionally substituted C₁₋₄ alkyl;
each R^{ss} is independently C₁₋₄ alkyl or phenyl optionally substituted by 1-3 groups R^{zz};
each R^{uu} is independently C₁₋₄ alkyl, phenyl optionally substituted by 1-3 groups R^{zz}, C₁₋₄ alkoxy, or NR^{ac}R^{ad};
each R^{zz} is independently halogen, C₁₋₄ alkyl, C₁₋₄ alkoxy, or C₁₋₄ alkylsulphonyl;
R^{ac} and R^{ad} are each independently hydrogen or C₁₋₄ alkyl;
or R⁵ and R⁶ together form a group ═C(Rⁱ)OR^{j}, ═C(R^{k})SR^{l}, ═C(R^{m})NRⁿR^{o} wherein
Rⁱ is hydrogen, C₁₋₄ alkyl, C₃₋₆ cycloalkyl, phenyl, C₁₋₄ alkoxy, C₁₋₄ alkylthio, or NR^{ac}R^{ad} wherein R^{ac} and R^{ad} are as defined above,
R^{j} and R^{l} are each independently C₁₋₄ alkyl,
R^{k} is hydrogen, C₁₋₄ alkyl, C₃₋₆ cycloalkyl, phenyl, C₁₋₄ alkylthio, or NR^{ac}R^{ad} wherein R^{ac} and R^{ad} are as defined above,
R^{m} is hydrogen, C₁₋₄ alkyl, C₃₋₆ cycloalkyl, phenyl, or NR^{ac}R^{ad} wherein R^{ac} and R^{ad} are as defined above, and
Rⁿ and R^{o} are each independently hydrogen or C₁₋₄ alkyl, or a salt form thereof.
